(19) United States
(12) Patent Application Publication
(10) Pub. No.: US 2004/0005582 A1
(43) Pub. Date: Jan. 8, 2004

(54) Biospecific desorption microflow systems and methods for studying biospecific interactions and their modulators

(75) Inventor: Edward Shipwash, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NanoBioDynamics, Incorporated, San Jose, CA

(21) Appl. No.: 10/327,531

(22) Filed: Dec. 19, 2002

Related U.S. Application Data
(63) Continuation-in-part of application No. 09/927,424, filed on Aug. 9, 2001.
(60) Provisional application No. 60/343,025, filed on Dec. 19, 2001.
(60) Provisional application No. 60/224,551, filed on Aug. 10, 2000.

(51) Int. Cl. C12Q 1/68; G01N 33/53; C12M 1/34

(52) U.S. Cl. 435/6; 435/7.1; 435/287.2

(57) ABSTRACT

Biospecific desorption microflow systems and methods employing immobilized prebound members of a binding pair are disclosed are used in detecting analytes in samples, identifying binding sites and studying biospecific interactions and their inhibitors on intact cells, cell membranes, cell organelles, cell fragments, proteins, and other biopolymers. The microflow reaction channel is in fluid connection with one or more reservoirs each having a means for transporting fluids or sample to a microflow channel having a prebound binding pair. The biospecifically desorbed labeled molecules may be continuously detected and quantitated on-line. Apparent dissociation constants and 1C50 values (for inhibitors) may be computed automatically. Fluorescent, luminescent, or electrogenic labels may be used to provide continuous flow microsystems having subpicomole sensitivities. Using microfluidic arrays, a single sample may be analyzed for the presence of multiple functional binding sites simultaneously. The method finds use as a universal technique for mapping the surfaces of proteins (epitope mapping) and other biopolymers for functional binding elements. The method is especially suitable for the functional analysis of the multitude of consensus sequences that are emerging from genome programs (for verification that a binding site predicted from a genome sequence is indeed functional) and for studying biospecific interactions that occur in the extracellular environment e.g. blood coagulation/fibrinolysis, inflammation, cell migration, bone biology, tissue and organ formation and regrowth. The method is well suited for studying biospecific interaction in an automated and highly controlled manner and for rapidly screening drug candidates for blocking these interactions.

### CROSS-REFERENCES TO RELATED APPLICATIONS

This application is a Non-Provisional of U.S. Provisional Application No. 60/343,025, filed Dec. 19, 2001 and this application is also a Continuation-in Part of U.S. application Ser. No. 09/927,424, filed Aug. 9, 2001, which claimed priority of U.S. Patent Application No. 60/224,551, filed on Aug. 10, 2000. The disclosures of each of the above applications are incorporated herein by reference.

### FIELD OF THE INVENTION

This invention relates to automated biospecific microscale desorption systems for studying biospecific interactions and binding sites of biopolymers (e.g., proteins, polynucleic acids) and modifiers thereof.

### BACKGROUND OF THE INVENTION

At the molecular level, essentially all biological functions are mediated through the selective binding of ligands and receptors. This selective interaction between ligands and their receptors is termed “biomolecular recognition.” In the past few decades, devices and systems applying biomolecular recognition phenomena have been developed for use in diagnostics, basic biological and pharmaceutical research, therapeutics, ligand/receptor detection and quantitation, and chemical analysis. The study and identification of ligands and receptors, including the sites and properties of the ligand-receptor interactions, is essential for a molecular understanding of biology and pathology. As a practical matter, the study of ligands, receptors, and their interactions has proven to be a highly fruitful path in the development of novel therapeutics, diagnostics, and other useful compositions and methods, including anti-microbials and pesticides.

Ligand-receptor or binding assays are powerful and well established in the prior art. Over the years the art has produced significant improvements in ligand assay design, reagents, and detection systems. The development of hybridoma technology and monoclonal antibody production resulted in immunoassays with improved specificity and sensitivity. In addition, phage display, combinatorial chemistry, antibody engineering, and directed evolution now make possible the production of antibodies, proteins, peptides, RNAs or oligonucleotides which bind virtually any desired molecule (e.g., biomolecules, modified amino acid residues on proteins, drugs, environmental pollutants, chemical warfare agents, pathogens, etc) with any desired affinity. In addition, antibodies can recognize conformational changes in proteins and other biopolymers.

Biochemists have also used the power of molecular recognition for the purification of biomolecules. Affinity chromatography, where a single biomolecule specifically and reversibly binds an immobilized ligand, can separate a biomolecule from an extract containing thousands of macromolecules in a single step. Biomolecules purified by affinity chromatography include antibodies and antigens, enzymes and inhibitors, regulatory enzymes, hormone-binding proteins, vitamin-binding proteins, receptors, lectins and glycoproteins, RNA and DNA (genes), bacteria, viruses and phages, cells, genetically engineered proteins, toxins, drugs, and others. The biomolecule bound by the immobilized ligand can often be eluted with a solution of the free biomolecule or another molecule which can compete for the binding site(s) of the molecule to be desorbed and eluted. Affinity elution is complimentary to affinity chromatography. In affinity elution, the specificity of interaction is at the stage of desorption from the support material, whereas in affinity chromatography the specificity occurs at the stage of adsorption.

In recent years, the development of miniaturized systems has revolutionized biochemical analysis. The development of miniaturized arrays and Lab on a Chip technologies represents a combination of several disciplines that include microfabrication, fluid dynamics, microfluidics, microelectromechanical systems (MEMS), chemistry, biology, physics, biophysics and engineering. These tiny gene chips, lab chips, and soon protein chips may become the standard platforms for biochemical, biomedical, toxicological and drug research and development as well as analytical chemistry. On-line microfluidic systems that transport liquid solutions in channels of micron dimensions have been used for high-throughput DNA genotyping (N. Zhang et al. (1999) Anal. Chem. 71, 1138-1145), polymerase chain reactions, and DNA sequencing reactions. Wooley, A. T. et al. (1996) Anal. Chem. 68, 720-723.

Results from massively parallel and quantitative gene expression measurements analyzing up to 40,000 genes at a time and whole-genome variant detection methods show the power and accuracy of combining biorecognition phenomena with miniaturized array based methods (Lipshutz, et al. (1999) Nat. Genet. 21: 20-24). Microarrays detect gene expression levels in parallel by measuring the hybridization of mRNA to many thousands of genes immobilized at high spatial resolution on a surface (Reviewed in Watson et al. (1998) Curr. Opin. Biotech. 9:609-614). Highly resolved detection is generally achieved by the laser induced fluorescence of a labeled probe. Capillary array electrophoresis, where many capillaries are run and detected in parallel, has recently been developed for rapid DNA sequencing (reviewed in Kheterpal and Mathies (1999) Anal. Chem. 71:31A-37A).

While microfluidics is not new, the potential applications and benefits in the life sciences, environmental chemistry, analytical and physical chemistry, toxicology, pharmacology, and biomedical engineering have not been realized. Many of the limitations of passive binding assays can be overcome by active microfluidic chips devices which facilitate the rapid transport, mixing and selective addressing of biomolecules to any position on the chip surface. Specially designed microsystems containing a multitude of sub-microliter chambers or microchannels may be used in combination with microfluidics and/or nano pipetting to analyze a multitude of samples simultaneously or nearly simultaneously.

Unfortunately, recent advances in rapid microscale gene analysis have greatly outpaced the study of biomolecular recognition events for proteins, other biopolymers, ligands, and other biological molecules in general. Present methods for mapping binding sites on proteins, carbohydrates, nucleic acids, polysaccharides and other biopolymers, for example, are comparatively slow, expensive, labor-intensive, and have not been automated. Rapid and sensitive methods are needed for mapping epitopes bound by antibodies. New methods and systems are needed for the experimental determination and characterization of biospecific interactions (protein-protein, protein-carbohydrate, antibody-antigen, protein-lipid, virus-cell, bacteria-cell, protein-drug, enzyme-substrate, enzyme-inhibitor, protein-DNA and protein RNA) including methods for determining the exact amino acid residues, nucleotide bases, or carbohydrate residues in polysaccharides, oligosaccharides or lipid molecules involved in each specific interaction as well as systems for high throughput screening for inhibitors of biospecific interactions.

Invented herein are biospecific desorption microflow systems that provide for these and other needs. These systems can be rapid, sensitive, inexpensive and suitable for automation, miniaturization and multiplexing as well as easy-to-use. Biospecific desorptions rely on the dissociation of biospecific binding partners and a detection method based typically on competitive displacement of pre-bound complexes with similar or equivalent binding sites during flow. Other forms of biospecific desorption may involve binding interactions with an allosteric site which alters the binding characteristics and desorption of the binding pair under study. Microflow biospecific desorption analysis can measure the interactions between two or more molecules by monitoring the desorption of an adsorbed binder caused by an analog of the binder free in solution. Biospecific desorption is successful when the interaction of the adsorbed molecule with the adsorbent is through one or more specific binding site(s), and it is possible to replace this interaction by free ligand in solution which has similar or equivalent binding site(s). This specificity makes this method suitable for mapping specific binding sites on the surfaces of proteins (e.g. which amino acids on the protein's surface are involved in binding) and other polymers (DNA, RNA, lipids, carbohydrates, synthetic polymers) and for otherwise identifying, quantifying, and characterizing the ligands, receptors/binders, and the biomolecular interactions, including allostery and conformational changes, involved in the biomolecular recognition events. The analysis can be accomplished over a wide range of affinity and sizes of both the immobilized and mobile binders. The analysis can be performed on a microscale dependent only on the limits of detectability of the binder eluting from the microchannel.

### SUMMARY OF THE INVENTION

The present invention provides biospecific desorption and affinity elution microflow systems, methods, and devices for studying specific molecular interactions under a variety of conditions, for mapping binding sites on the surfaces of biopolymers, for calculating apparent affinity constants, for detecting and measuring analyte(s) in sample(s), and for screening or identifying modifiers, ligands and binding pair members of specific biomolecular recognition interactions.

The microflow analytical devices of the present invention comprise first and second binding pair members. The first binding pair member(s) is immobilized to an area or surface of a chamber to be exposed to a flow stream or immobilized to a surface or portion of a channel for conducting the flow stream. The second binding pair member is reversibly bound to the first binding pair. In one embodiment, the immobilized binding pair member(s) may be in direct contact with the fluid of the flow stream. In another embodiment, the first or immobilized binding pair member is indirect contact with the flow stream and may be separated from the flow stream by a membrane that is permeable to one or more constituents of the flow stream. Such a constituent may be a binding modifier or a ligand or receptor of the immobilized binding pair member or the second binding member. In this case of indirect contact, the first binding pair member may be immobilized only by virtue of being separated by the flow stream by the permeable membrane. The flow stream controls the fluidic environment and conditions (buffer, modifiers, binding competitors, reagents, ligands, sample, etc) for studying the biomolecular interactions of the binding pair members and/or for detecting the competitive displacement or biospecific desorption of the second binding pair member. The effect measured may be an increase or a decrease in the amount or rate of desorption depending upon the configuration of the system and the binding pair members.

In one embodiment, the immobilized first binding pair member is covalently immobilized by attachment to a surface of a chamber or channel. In another embodiment, the first binding pair member is non-covalently attached to the surface. In other embodiments, the second binding pair member may be labeled with a detectable label and the labeled binder can be affinity eluted upon contact with a competing ligand or other modifier of the biomolecular interaction. The detectable label signals the presence or amount of a desorbed and eluted binder and thereby can indirectly provide a measure of the amount of the competing ligand or binder in a sample. In one embodiment, the labels are fluorescent labels.

In some embodiments, the biospecific desorption microflow system comprises a liquid flowing through a reaction microflow channel for transporting a sample; a receiving means for introducing at least one sample to the liquid stream; a flow control means for moving the liquid stream through the reaction channel; a binding pair or complex in fluid communication with the sample receiving means in which the sample is brought in contact with binding pairs or complexes and whereby a target mimicking the binding site on any of the binders in the pair or complex displaces the labeled binder; a detection apparatus connected to the reaction microflow channel for detecting any displaced binder which is released to the flow stream; and a waste reservoir or drain connected to the microflow reaction channel. In some embodiments, the microfluidic systems provide a microflow that is discontinuous. In other embodiments the microfluidic systems provide a microflow that is continuous.

In some embodiments, the microflow passages of the subject invention may be molded or machined into a substantially planar substrate such as a chip or cartridge. Or the microflow passages may be made from nonplanar materials (e.g., microcapillaries). The microflow passages may be straight, curved or coiled. The chip cartridge may be made from a variety of materials including but not limited to glass, silicon, quartz, or plastics that can be machined or molded to form microchannel passages. Microfluidic transport mechanisms such as pneumatic pumps and mechanical valves, centrifugal force, or electroosmotic pumps, or syringe pumps may be used to flow fluids from reservoirs through the microchannels. Otherwise flows may be achieved by gravity flow or capillary action without the use of a fluid transport device.

In some embodiments, a label need not be employed as the desorption of a binder may be detected by other methods such as the change in mass that results from the desorption of a binder. For example piezoelectric crystal devices or surface plasmon resonance based biosensors monitor mass changes and are suitable for use in the current inventions.

In additional embodiments, multiple parallel reaction channels are employed with spatially specific detectors (e.g., array detectors). In some embodiments, multiple samples are analyzed simultaneously or nearly simultaneously. By immobilizing different binding pair members or different binding pairs or complexes in each flow chamber multiple samples can be analyzed simultaneously for their effects on a plurality of different biomolecular recognition interactions. Alternatively the same binding member or pair may be analyzed in parallel flow channels to permit the simultaneous analysis of different conditions (e.g. different competitors, modifiers, or a range of different concentrations of the same modifiers or same binding pair members).

In one of its aspects, the invention is drawn to microflow methods for determining the temperature dependence of the binding between the binding members. In this aspect, the embodiments include a temperature regulating means to provide for adjusting or controlling the temperature of the locus of the binding events. In still further embodiments, the apparatus of the invention includes a temperature regulatory system or unit to adjust and or control the temperature of the biospecific desorption event under continuous or discontinuous flow conditions. For example, the microflow system may operate over temperature ranges from 4° C. to 40° C. The operating temperature ranges may be limited to the thermostability of the biomolecules or other binding members under study. For instance, higher temperatures can be employed to study the biomolecules of thermophilic microorganisms. In this case, one of the binding pair members is a biomolecule from a thermophilic microorganism showing increased temperature stability. The system can thereby study binding characteristics and desorption behavior over a correspondingly greater temperature range.

In other aspects, methods of the invention are used to conduct a microflow thermodynamic analysis of ligand binding, including for example, the molecular events and chemical changes involved in ligand-receptor, drug-receptor, or inhibitor-receptor interactions. These thermodynamic methods can be applied to study all binding events. Such methods include, for example, microflow methods of conducting thermodynamic analyses by determining the binding pair or complex dissociation constant or apparent dissociation constant at various temperatures. Temperature-related changes in these constants can be used to derive using standard physical chemical relationships the standard free energy (ΔG°), enthalpy, (ΔH°), and entropy, (ΔS°) of the binding event using the integrated form of the van't Hoff equation which relates the dissociation constant with temperature.

In one of its aspects, the temperature regulated microflow systems and methods of the invention are used to identify structural and/or functional differences between binders. In particular, the systems and methods can be used to identify or distinguish isoforms of similar binders (e.g., alternatively spliced or co- and post-translational modified forms of binders including drug-receptor interactions). This method can also be applied to the field of proteomics to detect or identify a multitude of alternatively spliced and modified protein or polypeptide forms, resulting from limited proteolysis, phosphorylations, sulfations, oxidation, etc, as well as any such changes occurring in disease states. In some embodiments, therefore, a plurality of members of a family of related (e.g., structurally or functionally similar) but variant first binders are each immobilized in separate microchannels or different location of known address and the second binder is the same for each immobilized binder. In other embodiments, the first binder is the same and immobilized in each of a plurality of microchannels and the second binder is a member of a family of structurally or functionally similar but variant binders so as to provide a plurality of microchannels each having different variant binder complex at a known address.

This method can be used to detect proteins or polypeptides in which one or more amino acids have been altered. Dissociation constants of chemical and biochemical reactions typically vary with temperature. No difference in the temperature dependency of dissociation constants is observed for two protein isoforms, receptor subtypes, or a mutant or defective protein for their cognate binding partner(s) if they are the same. But if mutants, isoforms, damaged proteins, or receptor subtypes exist as separate functional entities (e.g.,—resulting from point mutations, isoforms, modified proteins, alternatively spliced subtypes, etc), then the temperature behavior of the two dissociation constants or apparent dissociation constants usually differ. In some embodiments of the invention, the binding members therefore have a plurality of variant protein binding members whose binding characteristics are to be compared by thermodynamic or other means (e.g., allosteric competition).

In some embodiments, the microflow methods provide a rapid analysis of mutant proteins. In these embodiments, at least one of the binders is a mutant protein or polypeptide and at least one of the binders is the wild-type or normal protein. The value of the apparent equilibrium constants as a function of temperature can be used to derive a profile for a known active protein which may be used as a control. The control profile is then compared to the corresponding temperature profile for the suspected subtype, mutant, isoform, etc. There are many known methods to measure the binding constant of molecular complexes. A change in the measured property (biospecific desorption) as a function of the ligand concentration is typically employed in the quantitative measurement of the binding constant. Different concentrations of free labeled binders are typically employed (e.g., 10-2000 pM) in these embodiments of the method.

In another aspect, the invention provides biospecific desorption microfluidic analytical devices configured as microdialysis or ultrafiltration probes to be implanted into living animals. In some embodiments, these devices can be implanted in mammalian organs and tissues such as liver, lung, heart, kidneys, brains, as well as cells such as nerve cells, egg cells, and isolated tissues. Such biospecific desorption systems may bear a labeled analog to the analyte to be detected and or quantified. In these embodiments the labeled analyte analog can be bound to its cognate binder which is immobilized on a transducer or a surface in contact with a transducer (e.g., optical fiber, optical particle or electrode). Such transducers are known in the prior art.

The label can be appropriate for the nature of the transducer and detector. For example, optically detectable labels such as fluorescent dyes are used with optical fiber based systems whereas electrochemical labels such as ferrocine or enzyme labels along with their substrates are used for electrochemical based detectors. These labels and methods for their detection as well as a multitude of others are well known in the prior art.

Preferred embodiments have binding members or analytes to be detected and/or quantitated that are drugs, drug candidates, toxins, biomolecules, hormones, neurotransmitters, metabolites, amino acids, chemical and biochemical warfare agents, and environmental pollutants.

In another aspect, biospecific desorption based analytical probes may be placed in the environment being analyzed (e.g., soil, water sources (groundwater, streams, lakes, oceans and the like). For biospecific desorption analysis in remote locations (i.e. where the detector is some distance from the detector, light source, and computers such as measuring environmental samples) optical fibers are preferred transducers. In some embodiments, a binding member is an industrial chemical, a chemical warfare agent, a biological warfare agent, a microbiological agent, or other environmental pollutant.

In another aspect, the microflow devices are used in cell culture systems especially plant and animal cell culture systems. The devices may be used to monitor the culture medium for metabolites, cellular products, and chemical indicators of cellular growth and activity.

In another aspect, the microflow systems have a first immobilized binder which is a functional biomacromolecule at a concentration which is comparable to that of a second binder.

In another aspect, the invention provides methods for detecting the biospecific desorption of ions. In these embodiments, the binding pair members are capable of releasing a proton (e.g., biospecific binding events result in the desorption of protons). If these protons are detected, the binding of a cognate binding partner may be detected and quantitated. The method and systems are not limited to any single method of detecting protons. Many methods are available to detect changes in proton concentration. For example, pH meters are well known in the art. The detection of protons within cells and Microsystems are also known. Protons are released when DNA binding proteins bind to DNA and when complimentary nucleic acids hybridize.

In some embodiments, a binder is a proteinase or a proteinase inhibitor. More particularly the binder may be a serine proteinase or a serine proteinase inhibitor.

In a further embodiment, microflow biospecific desorption is employed for the conformational analysis of proteins and other macromolecules. It is highly desirable to have a method for detecting different conformational states in proteins and nucleic acids. In one embodiment, antibodies which are specific to certain conformations of proteins are used as binders to their conformation-specific proteins. Proteins having conformations which are the same as the bound proteins can be contacted with the bound conformational isoform causing a biospecific desorption which can be detected. In like manner, in other embodiments, conformation specific antibodies can be employed as binders to detect conformation specific nucleic acids such as RNAs.

In further embodiments, microflow biospecific desorption is employed to detect affinity tags, labels, and chemical cross-linking reagents. Antibodies to these chemicals are bound with their labeled binder (e.g., a chemical crosslinking molecule). When a biopolymer such as a protein or fragment thereof containing the cross-linking molecule is contacted with the bound labeled crosslinking reagent, a biospecific desorption can take place allowing the protein fragment bearing the crosslinking group to be detected. The same embodiment can detect any protein or other biopolymer to which a crosslinker or other chemical tag is attached.

In other embodiments, the binding pair members (e.g., receptors and their corresponding ligands) may be automatically monitored without the need for immobilization in continuous flow systems employing fluorescence techniques such as fluorescence polarization, fluorescence energy transfer, or fluorescence correlation spectroscopy as a detection method. In these embodiments, a fluorescently labeled prebound binding pair is provided. The labeled prebound binding members can flow from a reservoir into a main microflow channel. The main microflow channel can be in fluid connection with an array of reservoirs. Each unique reservoir in the array can contain a different putative modifier, inhibitor, or competitor of the receptor—labeled ligand pair binding. Fluids from each reservoir can be perfused (flowed) through the main channel and mixed with the flow of the prebound labeled binding pair members. Inhibitors or molecules or other entities that block the biospecific interaction of the binding pair members can be the targets to be identified by desorption of the labeled binding member. This desorption can provide a proportionate change in the fluorescent polarization of the desorbed fluorescently labeled ligand.

Some analytical devices according to the present invention may also comprise a plurality of channels in fluid communication with reservoirs and having a means to transport fluids and fluid-borne substances from the reservoirs through the main reaction channel bearing the binding complex(s).

In some embodiments, an array of reservoirs is in fluid connection with the channel bearing the binding complexes. In some embodiments reservoirs in the array have different samples of potential inhibitors of the biomolecular binding interaction taking place in the reaction channel. Other embodiments may provide reservoirs with different buffers, for example, different buffers for optimizing the binding affinities to facilitate a biological desorption assay. In other embodiments, the buffers contain different reagents or different concentrations of reagents such as co-factors, metals, proteins, protein domains, protein motifs, peptides, anions, cations, antibodies or antibody fragments, carbohydrates, lipids, nucleic acids, heparin, drugs, anticoagulants and the like so that the dependence of the binding complex of interest on these substances can be studied in an integrated and automated fashion. Different reservoirs may also contain the same sample at different concentrations. The location of the samples in the reservoir array can provide an address for later reference to identify the substance causing the observed effect, for example, inhibition of the biospecific interaction.

In some embodiments different binding pairs or complexes may be immobilized on distinguishable beads or microspheres. The beads may be, for example, of different fluorescent color. In these embodiments, the binding pair may be identified by the characteristic of the bead (e.g. color, or size) and the binding state of the binding pair in the presence of a potential inhibitor may be determined by fluorescence techniques as described herein.

In some embodiments, computer-controlled, integrated biospecific desorption microsystems are envisaged in which a series of reagents, peptides, oligonucleotides, drugs, cells, and other substances are perfused through the microsystems. In further embodiments, miniaturized autoinjectors may be employed. Integrated microfluidic transport systems may deliver reagents and biomolecules from reservoirs through the microflow system in a highly controlled manner.

In one embodiment, a miniaturized flow system is provided in which a labeled substance is adsorbed within the flow stream in such a way that the labeled substance can be eluted biospecifically or captured by another substance. The micro flow system can have at least one sample inlet and adsorbed labeled analyte analogue and integrated detector. The biospecific interaction can be monitored by following the elution of labeled analyte analog.

In one of its aspects, a biospecific desorption microflow systems of the invention are configured to study biospecific interactions and their modifiers (e.g., inhibitors) including interactions between antigens and antibodies, enzymes and inhibitors, hormone binding proteins and hormones, vitamin binding proteins and vitamins, drug binding proteins and drugs, bacteria, viruses, phages, and cells.

In one aspect, a displacement competition microflow system studies biomolecular recognition interactions of biopolymer binding pair members. A sample fluid is transported by microfluidic means to a reaction microchannel having at least one first biopolymer reversibly bound through specific recognition sites to a second biopolymer wherein the first biopolymer is immobilized irreversibly to a solid support and the second biopolymer is labeled with a detectable tag. In one embodiment, the tag is fluorescent. In one embodiment, the system is used in mapping functional sites (binding sites) on proteins and other biopolymers (e.g., polynucleotides, polysaccharides, polypeptides).

In another aspect, embodiments provide competitive displacement microarray systems for studying the interactions of biospecific binding pair members and modifiers or inhibitors thereof. In some further embodiments the binding pair members are biospecific receptor-ligand, protein-protein, protein-nucleic acid, protein-carbohydrate, cell-cell, virus-cell, cell-extracellular matrix, and cell-substratum interactions. In some embodiments, the immobilized receptor is a receptor involved in cellular adhesion (e.g., cell-cell, cell-virus, and cell-extracellular matrix adhesion). In further embodiments, these receptors are selected from the group comprising integrins, selectins, cadherins, immunoglobulin superfamily members, mucins, leucine rich glycoprotein, CD36, CD44 family members and others.

In some embodiments, the binding pair members comprise adhesion biomolecules and model the adhesion of microorganisms to inanimate and biological surfaces. These embodiments allow the study and identification of inhibitors and modifiers of such adhesion. Further embodiments are directed toward binding pair members modeling bacterial adhesions in host/pathogen interactions in animals, the accumulation of organisms on the teeth, or binding pair members modeling other biological or nonbiological adhesions. These embodiments can be used to identify inhibitors or modifiers of such adhesions.

In some embodiments, the binding pair members comprise a biomolecular recognition molecule or binder (antibody, oligonucleotide apatamer, protein, or other biomolecule) that specifically and reversibly binds modified groups on proteins. The biomolecule may be employed as a the immobilized binding entity of the invention. Labeled analogs to the modified group may be reversibly bound to the immobilized binder to be exposed to a sample. In further embodiments, the biorecognition molecules recognize one or more of the modifications selected from the group consisting of phosphorylated residues, (e.g., tyrosine phosphate, serine phosphate, arid threonine phosphate), lipid modified residues (e.g. as on lipid modified proteins), glycoproteins, sulfation modifications of residues, N-myristolyation, and N-terminal modifications of proteins or peptides. In some embodiments, the immobilized biorecognition binding pair member is an immobilized antibody. In further embodiments the antibody recognizes a ligand selected from the group consisting of N-myristate, N-formyl-, N-methyl, N-acyl, or N-aminoacyl modifications. In further embodiments, the immobilized antibody recognizes one or more of the modifications selected from the group consisting of phosphorylated residues, (e.g., tyrosine phosphate, serine phosphate, arid threonine phosphate), lipid residues (e.g. as on lipid modified proteins), (antibodies against specific lipids), glycoproteins (antibodies against specific carbohydrates), sulfation, antibodies against tyrosine sulfate,

In one of its aspects, the invention provides a microflow system and method employing biospecific desorption for mapping functional binding sites on the surfaces of proteins and nucleic acids comprising the steps of: (a) providing a binding pair or complex in a microflow reaction channel or capillary wherein one member of the pair or complex is immobilized in the flow passage (by covalent or noncovalent, e.g. biotin-avidin technology) and the other member of the pair or complex is labeled (e.g. with a fluorescent tag); (b) flowing a liquid sample containing biopolymers (e.g. peptide, oligonucleotides) corresponding to binding sites on the binding pair or complex through the reaction channel; one or more samples, each comprising a different biopolymer are flowed, one at a time, through the microflow passage bearing the binding complex; (c) allowing biopolymers corresponding to the binding sites on the binding pair or complex to biospecifically desorb (competitively displace) the binders, (d) detecting the displaced binder(s) with a detector, and (e) identifying the binding sites on the protein/and or nucleic acid from the known sample causing the biospecific desorption.

In another aspect, this invention provides a microflow system and method employing biospecific desorption to screen for inhibitors of biospecific interactions (e.g. protein-protein, virus-cell, protein-cell, protein-nucleic acid, antibody-antigen, etc) comprising the steps of: (a) providing a binding pair or complex in a microflow channel or capillary wherein one member of the pair or complex may be labeled; (b) flowing a liquid sample containing a possible inhibitor of the biospecific interaction in the microflow reaction channel through the reaction channel; one or more samples, each containing a different potential inhibitor are flowed, one at a time, through the reaction channel. In some embodiments each sample is transported from a unique reservoir through the reaction channel; (c) allowing samples to desorb the binders; (d) detecting the desorbed binder(s) with a detector; and (e) identifying the inhibitor from the known sample causing a desorption and thereby inhibiting the biospecific interaction.

In another aspect, the invention provides a microflow system and method employing biospecific desorption to identify co- and post-translational modifications on proteins comprising the steps of: (a) immobilizing a binder (antibody, receptor, aptamer) that specifically and reversibly binds a modified amino acid in a microflow reaction channel; (b) binding a labeled analog of the modified amino acid (e.g. a fluorescently labeled peptide bearing a tyrosine phosphate bound to an immobilized anti-tyrosine phosphate antibody) to the immobilized binder; (c) flowing a sample containing the protein or fragment thereof to be analyzed through the reaction microchannel; (d) detection the biospecifically desorbed labeled analog with a detector; and (e) identifying the modified amino acid from the biospecific desorption of the labeled analog.

In another aspect, the invention provides a microflow system employing biospecific desorption for epitope mapping comprising the following steps (a) immobilizing an antibody or protein antigen in a microflow channel (b) binding the protein antigen or antibody which may be fluorescently labeled to the immobilized cognate binder (c) flowing one or a series of samples each containing a unique peptide corresponding to a different portion of the amino acid sequence of the protein antigen through the reaction channel one at a time; a set of overlapping peptides patterned on the amino acid sequence of the protein antigen is hence flowed through the reaction channel, one at a time (d) detection of the biospecifically desorbed labeled binders with a detector, and (e) identifying the epitope on the protein from the peptide causing the biospecific desorption.

In another aspect, the invention provides means for identifying new therapeutic agents for HIV. In one embodiment, the invention provides microflow systems for high throughput screening of inhibitors of HIV-cell interactions which enable HIV viruses to gain entry into cells. In other embodiments, the invention provides immobilized binding pair member(s) that are target cell components involved in the adhesion or infection of target cells by HIV virus. In some embodiments, an immobilized binding pair member comprises at least one receptor on the host cell surface which is involved in the attachment of the HIV virus to the cell surface. In some embodiments, these binding pair members can include the CD4 receptor as well as a chemokine receptor, particularly a member of the G-protein coupled 7TM superfamily, and glycoproteins such as 120 (gp120).

In another aspect, the invention purposefully and counter-intuitively introduces nonspecific binding to allow the study of the specific binding interactions involved in a microflow biospecific desorption system. In some embodiments, a supporting matrix is employed to increase the retention of a binding pair member in an immobilized complex. In other embodiments, appropriate buffer conditions are provided to strengthen weak binders and weaken strong binders, in addition to using high ligand load to increase the retention of weak binders.

Although major applications are believed to be in the area of biospecific interactions and their modifiers, the inventive methods and devices have diverse additional applications. For instance, the systems may be used to screen for substances such as toxins or environmental contaminants in a sample or study the binding of any compounds to other materials. For example, the system may analyze the desorption of pesticides adsorbed onto clay and other soil components. In this case a labeled pesticide or other pollutant that adsorbs to clay may be used and the clay having an adsorbed labeled pesticide placed in a flow channel. Potential agents that may cause a desorption of the pesticide from the clay may be perfused through the flow channel as described and those causing a desorption of the labeled pollutant may be identified by using the same methods as those for identifying inhibitors of biospecific interactions.

Other features, objects and advantages of the invention and its preferred embodiments can become apparent from the detailed description and claims which follow.

### BRIEF DESCRIPTION OF THE DRAWINGS

FIG. 1 is a schematic illustration of biospecific desorption in a microflow channel.

FIG. 2 is a schematic illustration of biospecific desorption in a microflow channel using an optical detector and fluorescent labels.

FIG. 3 is a schematic drawing of a continuous microflow system employing biospecific desorption and optical detection of the desorbed binder.

FIG. 4 is a schematic illustration of a microflow systems configured to study protein-protein interactions.

FIG. 5 is a schematic illustration of a microflow system having a promoter immobilized in a flow chamber for studying protein-nucleic acid interactions-rapid promoter analysis.

FIG. 6 is a schematic illustration of a microflow system providing miniaturized continuous flow displacement assays as a universal technique for mapping functional sites in proteins and other biopolymers.

FIG. 7 is a schematic illustration of a microflow system to study protein-protein interactions using a competitive displacement desorption to detect a modified protein residue by use of a modification-specific antibody.

FIG. 8 is a schematic illustration of a microflow system having automated high throughput screening microsystem using continuous biospecific desorption for the isolation of antibodies having desired affinity properties.

FIG. 9 is a schematic illustration of a microflow system to study protein-protein interactions.

FIG. 10 is a schematic illustration of a microflow system to study protein-protein and drug interactions related to viral diseases as exemplified by AIDS.

FIG. 11 is a schematic illustration of a microflow system for epitope mapping using microflow biospecific desorption.

FIG. 12 is a schematic illustration of a microflow system for high throughput screening of chemicals such as drugs or, as exemplified, peptides.

FIG. 13 s is a schematic illustration of a microflow system using a homogeneous fluorescent binding assay to detect inhibitors of cell surface receptor-ligand interactions.

FIG. 14 is a schematic illustration of a microflow system for the automated analysis of the inhibition of biospecific interactions using two labels and fluorescence detection.

FIG. 15 is a schematic illustration of a microflow system of an automated microsystem suitable for screening for inhibitors, activators, or co-factors of biospecific interactions using an energy transfer assay. The ligand and receptor are labeled with an energy donor and acceptor.

FIG. 16 is schematic drawing of a microflow system employing integrated fluorescence polarization to detect the inhibition of ligand-receptor interactions. One binder is immobilized on a bead, phage, vesicle, cell, nanoparticle or the like and bound to a labeled ligand. Inhibitors are perfused through the reaction channel one at a time from a separate reservoir.

FIG. 17 is a schematic representation of a microflow system for studying cell to cell interactions as exemplified by neutrophil and monocyte adhesion to endothelial cell in a microflow channel.

FIG. 18A is a schematic depiction of a rapid automated microfluidic chip for determining the presence and/or amount of a receptor to a drug or hormone in a sample using biospecific desorption during flow.

FIG. 18B. depicts a rapid automated microfluidic chip for determining the presence and/or amount of a hormone in a sample.

FIG. 19 is a schematic drawing of a microflow system employing integrated fluorescence polarization to detect the inhibition of ligand-receptor interactions. One binder is immobilized on a bead, phage, vesicle, cell, nanoparticle or the like and bound to a labeled ligand.

FIG. 20 is a schematic illustration of a microflow system to study cell-protein interactions in microflow systems using biospecific desorption and flow detection.

FIG. 21 is a schematic illustration of a microflow system for high through put drug screening. This integrated microsystem is computer-controlled so that a series of drugs or other substances can be perfused through the main microchannel bearing the biospecific interaction.

FIG. 22 is a schematic illustration of a microflow system for studing cell-cell interactions in a microflow system.

FIG. 23 is a schematic illustration of a microflow system for the analysis of protein-cell interactions.

FIG. 24 is a schematic illustration of a microflow system for the analysis of cell-virus interactions in a microflow system.

FIG. 25 is a schematic illustration of a microflow system for epitope mapping using microflow biospecific desorption.

FIG. 26 is a schematic drawing of an integrated microflow system suitable for automated screening of inhibitors of biospecific interactions using integrated fluorescence polarization as a detection assay.

FIG. 27 is a schematic illustration of a microflow system for studying cell-cell interactions.

FIG. 28 is a schematic illustration of a microflow system for studying cell-protein interactions.

FIG. 29 is a schematic drawing of three microflow systems having an electrode biosensor, optical biosensor, or an surface plasmon biosensor respectively.

FIG. 30 is a schematic illustration of a microflow system as applied to allosteric binding events.

### DETAILED DESCRIPTION OF THE INVENTION

Unless otherwise defined, all technical and scientific terms used herein have the same meaning as commonly understood by one of ordinary skill in the art to which this invention belongs. Each publication, patent application, patent, and other reference cited herein is incorporated by reference in its entirety to the extent that it is not inconsistent with the present disclosure.

As used in this specification and the appended claims, the singular forms “a,” “an,” and “the” include plural reference unless the context clearly dictates otherwise.

Utility

The invention has many advantages over conventional and other microfluidic techniques. The invention detects the displacement or desorption of a prebound binding pair member from its complementary binding pair member. Contact with a modifier or competitor of the biomolecular interaction between the binding pair members alters the rate and/or amount of displacement. The displaced ligand or receptor is then detected. The time period for conducting this microflow process can be much shorter than other microflow and conventional techniques. The method does not require that a steady state equilibrium between competing ligands be established. The time course and extent of the displacement can serve as a measure of the ability of a sample to interfere with the biomolecular interaction of the binding pair members and indirectly indicate the presence and amount of an analyte therein.

Conventional separation techniques are generally manually intensive. In some embodiments, the present invention only requires sample introduction since the remaining processing is automatic. Since reactions are typically in the liquid phase, the methods allow greater speed, greater specificity and less background biochemical noise; and quantitation is achieved in tens of minutes instead of hours.

Microflow systems are well suitable for automation and multiplexing allowing the analysis of multiple samples simultaneously. As a microflow system, the operation of the invention requires only small amounts of sample greatly conserving materials and avoiding downstream waste. For example, reagent reservoirs may have volumes ranging from 0.01 to 100 microliters, more typically, 0.1 to 10 microliters. Once drawn from the reservoir, sample volumes transported through the microchannels can be as small as from 1 to 1000 nanoliters, and more typically, 10 to 400 nanoliters. Volumes of sample drawn for individual microinjected reaction or separation plugs may be as small as 0.01 to 200 nanoliters, and more typically, 0.1 to 40 nanoliters.

Moreover, in some embodiments, the system can be easily regenerated by subsequently contacting the immobilized member of the binding pair with a sufficient amount of the second binding member so as to provide a regenerated binding pair for conducting a second determination on a sample. In other embodiments, the microflows system allows for the processing of a plurality of samples simultaneously.

The methods can be sensitive, rapid, and efficient. In preferred embodiments, the methods are capable of studying multiple samples or detecting multiple functional elements simultaneously. The microflow systems consume tiny amounts of sample and reagent. The methods can be performed without incubation and washing steps or the introduction of indicator reagents following sample loading. Many samples can be rapidly analyzed by the use of a single aliquot of immobilized capture element bound to the labeled analyte analog. Intact cells and intact proteins may be analyzed.

These systems may be especially useful in studying protein-protein interactions for extracellular proteins and for studying biospecific interactions in a highly controlled and easily changeable microenvironment. The current art has produced rapid methods for studying protein-protein interactions in yeast two hybrid systems. These other systems are suitable for studying protein-protein interactions in living yeast cells. However, these other systems are not suitable for studying protein-protein interactions for extracellular proteins. Furthermore, these other systems ignore the microenvironment. Biospecific interactions are dependent on the microenvironment. Human proteins expressed in yeast are exposed to a multitude of yeast proteins and a microenvironment different than what they would encounter in human cells. This may lead to artifactual binding in the other systems. Thus, all binding partners identified in yeast systems may need to be confirmed using other methods. The methods invented herein may provide a means for the rapid confirmation of these binding interactions in a controlled environment on a microscale.

Many protein-protein interactions of great biological and biomedical importance occur on the cell surface or in the extracellular environment (e.g. in blood or the extracellular matrix). In the microflow systems invented herein, the microenvironments may be controlled and changed at may. Drugs, peptides, and other substances can be automatically and sequentially perfused through these microflow systems and their effects on biospecific binding may be continuously detected and recorded. This may provide an ideal platform for rapid and automated experimentations on a microscale.

For example, a multitude of protein-protein, protein-cell, cell-cell, protein-carbohydrate, protein-lipid interactions occur in blood coagulation, immunological, wound healing, and developmental pathways in all multicellular organisms. These biospecific interactions cannot be studied in a yeast or bacterial cell.

Current methods for analyzing co- and post-translational modifications employ mass spectrometry are not suitable for the analysis of large biomolecules especially intact proteins. In addition, these methods cannot be used with intact cells, organelles and the like. Indeed, mass measurements are of no use for analyzing functional motifs. Continuous biospecific elution micro flow systems are the preferred platforms for the analysis of co- and post-translational modifications of proteins and other biopolymers.

The microflow systems invented herein are useful for studying biospecific interactions such as antibodies and antigens, enzymes and inhibitors, hormone-binding proteins, vitamin-binding proteins, receptors, lectins and glycoproteins, RNA and DNA, bacteria, viruses and phages, and cells.

The method is analogous to the ability of an affinity column to mimic the recognition of a soluble ligand. Elution of an immobilized binder under nonchaotropic buffer conditions allows a dynamic equilibrium between association and dissociation. It is dependent on the equilibrium constant for the immobilized binder-free binder interaction. Therefore, affinity is reflected in the elution volume. The analytical use of affinity chromatography has been demonstrated (Dunn, B. M. and Chaiken, I. M. (1974) “Quantitative affinity chromatography. Determination of binding constants by elution with competitive inhibitors.” Proc. Natl. Acad. Sci. USA 71, 2382-2385; Swaisgood, H. E., and Chaiken, I. M. (1985) in “Analytical Affinity Chromatography”, (Chaiken, I. M., Ed), CRC Press, Boca Raton, Fla., pp. 65-115).

Typically binding assays use radioactive ligands, 0.5 ml volumes and at least a 30-minute incubation times. High concentrations of ligands are needed. The methods invented herein use tiny volumes microliter-picoliters and continuous flow thereby eliminating the incubation times. Furthermore, these methods use ultrasensitive fluorescence or electrochemical detectors that may exceed radioactive labels in sensitivity by orders of magnitude.

Biospecific Desorption

“Biospecific desorption” refers to the displacement or desorption of one member of a binding pair or one or more members of a multicomponent complex of molecules upon contact with another molecule or substances which can compete with or otherwise inhibit (for example, specifically binding to a macromolecule and causing a conformational change) the binding of the desorbed member with the other member of the binding pair or complex. The biospecificity is inherent in the binding preferences of the binding pair or complex members. Biospecific desorption is related to affinity elution in some aspects and is complementary to affinity chromatography in that the specificity of the interaction is at the stage of desorption from the support material or complex whereas in affinity chromatography specificity occurs at the stage of adsorption. The principals of biospecific desorption serve as the basis of novel methods of detection and analysis and are employing microflow systems for the rapid analysis of binding elements on a microscale.

Biospecific desorption can be different from competitive displacement in the case that the biospecific desorption event is not due to a competitive displacement but may, for example, be caused by the specific recognition or binding to a region other than the ligand binding site. Binding complexes may include two or more binders. Many biochemical complexes are comprised of multiple binders which may include proteins, RNAs, lipids, vesicles, polysaccharides, metals, ions, organic acids, co-factors, and the like. One or more members of a binding complex may be biospecifically desorbed and detected.

In many cases a biospecific binder e.g., inhibitor or activator, does not show a close similarity to that of the ligand-receptor binding site but instead specifically binds to another site. This site may be known as the allosteric site. The inhibition of a biospecific interaction may result from a distortion of the three-dimensional structure of one or more of the biomolecules in the binding complex which can be caused by the binding of an inhibitor. This distortion may be transmitted to the ligand-receptor binding site even though the inhibitor or activator binds far from that site. In some cases two or more distinct conformations of the biomolecules may exist, one binding ligands well and the other binding ligands poorly or binding inhibitors well or poorly. Biospecific adsorption to an allosteric site may increase the binding affinity of a binding pair or complex and this can occur because the activator stabilizes the conformation that binds the cognate binders best. The quantitative treatment of such activation is similar to that of inhibition. Allosteric inhibitors and activators may be considered together and can be considered as modifiers or modulators. The binding of a substance to an allosteric site with the introduction of conformational changes forms the basis of a multitude of bioregulary aspects. The term allostery may be used to the effects of allosteric modifiers, which may be either inhibitors or activators of biospecific binding on oligomeric biomolecules or polymers including biopolymers. Monomeric biomolecules biomolecules may also be subject to allosteric by modifiers.

The simple combination of multiple conformations with different binding properties provides a means by which biospecific interactions may be turned “on” or “off” in response to changing conditions. This forms the molecular basis for metabolic control and occurs throughout all of living organisms and cells. Indeed, probably the most common and widespread control mechanisms in cells are allosteric inhibition and allosteric activation. Allosteric control may also be widely used in the extracellular environment, e.g., in blood, and the extracellular matrix. Often biomolecules, especially proteins, exist as two or more isoforms. Only one isoform may be inhibited by a particular substance. Whereas different substances may inhibits other isoforms. Regulatory subunits are widely dispersed in biomolecules. The binding of inhibitors or activators to the specific sites on the regulatory subunits often induces a conformational change altering their interaction with the binding partner. Biospecific desorption may also be caused by a conformational change in a protein caused by post translational modifications. For example, the phosphorylation of certain amino acids on the proteins by protein kinases often induces a conformational change in the binder which inhibits or promotes binding to its ligand. For example tyrosine protein kinases phosphorylated certain tyrosines on certain proteins which can inhibit or activate specific binding. Other kinases phosphorylated serine residues and still others phosphorylated serine. This differential binding can be monitored using biospecific desroptioin as a detection method. Limited proteolysis is a regulatory mechanism which changes the binding preferences for protein-ligand, especially protein-protein interaction. Limited proteolysis is biospecific and may promote a biospecific desorption of prebound binders.

The systems invented herein can be used to study biospecific desorption caused by post translational modifications such as phosphorylations, biospecific limited proteolysis, and allosteric systems as well as others. The effect on desorption may be in any direction (e.g., to decrease or increase the rate or amount of desorption).

In the case of a complex of two binders, A and B, the binding is generally assumed to occur as a reversible bimolecular reaction:

A+B⇄AB

The free energy change for this reaction is given by the sum of the standard free energy change and terms relating the activity (or concentration) of each binder under the given conditions to the standard value by:

ΔG=ΔG°+RT ln(A)(B)/(AB)

in the above textbook formula R is the gas constant and T is the absolute temperature.

At a given temperature, the change in free energy of this reaction is a constant and under the conditions of equilibrium i.e., ΔG=0 the activity constant is also constant and termed the equilibrium constant K_{eq}. It is convenient in biochemistry to use the reciprocal of K_{eq}, the dissociation constant, K_{d}. Under these conditions equation 2 becomes ΔG=ΔG°+RT ln K_{d}

The variation of K_{d} with temperature, using the relation between change in free energy and changes in enthalpy and entropy, ΔG°=ΔH°−T ΔS°, is described by the integrated form of the van't Hoff equation: ln Kd=ΔH°/RT−ΔS°/R. From this a plot of ln K_{d} against 1/T gives a theoretically straight line with slope ΔH₀/R and y intercept—ΔS/R. The ln K_{d} decreases as the temperature increases if ΔH° is positive (endothermic reaction) and ln K_{d} increases as the temperature increases if ΔH° is negative (exothermic reaction).

Until recently, it was generally accepted that for good specific binding K_{d} must be less than about 0.003 mM, or 0.000003M. This is substantially smaller than most protein-ligand dissociation constants. Considering that the specific interactions with an immobilized ligand in a flow passage is likely to be weaker than the free ligand (due to steric constraints) one may ask how affinity chromatography or biospecific desorption can ever work. Surprisingly, the answer to this question is found in the purposeful introduction of nonspecific binding in the supporting matrix and using appropriate buffer conditions to strengthen weak binders and weaken strong binders, in addition to using high ligand load to increase the retention of weak binders.

The retention of interacting substances in a flow passage depends of the amount of specific binders, the affinity or avidity between the specific binders, and the physical characteristics of the matrix. Avidity describes the multivalent binding between multiple bind binding sites.

In recent years, we have experienced a growing awareness of the importance of weak and rapid binding events governing many biospecific interactions. Examples include protein-peptide interactions, (Fairchild, P. J and Wraith, D. C. (1996) “Lowering the tone: mechanisms of immunodominance among epitopes with low affinity for MHC (Immunol. Today 17, 80-85) virus-cell interactions, (Haywood, A. M. (1994) Virus receptors: Binding, adhesion strengthening, and changes in viral structure. J. Virol. 68, 1-5), cell adhesion and cell-cell interactions (Hakomori, S.-I. (1993)“Structure and function sphingoglycolipids in transmembrane signaling and cell-cell interactions.” Biochem. Soc. Trans. 21, 583-595; van der Merwe, P. A. et al (1993)“Affinity and kinetic analysis of the interaction of the cell adhesion molecules rat CD2 and CD48. EMBO J. 12, 4945-4954.

By implementing weak affinities under high immobilized ligand load significant retention of weakly interacting biospecific binders can be obtained. One of the drawbacks of the current art methods for analyzing weak interactions is that large amounts of binders (for example 10-100 milligrams of a protein is often employed to study weak affinities). However, retention is proportional to the concentration and not to the absolute amount of ligand. The systems invented herein can maintain the high concentration level of the active ligand using submicrogram amounts of protein compared to the ten's of milligrams needed using current methods.

Surprisingly, specificity can be accomplished in biological systems despite the fact that individual interactions are in the range of K_{d}=0.002M-0.003M or less. Bioaffinity chromatography has recently been achieved in the 0.01 M range of K_{d}. Leickt, L et al (1997) “Bioaffinity chromatography in the 10 mM range of K_{d}” Analytical Biochemistry 253, 135-136. In these cases biomolecular recognition is achieved is achieved by multiple binding either in a form of repeated binding events or by multivalent binding involving several simultaneous weak binding events. The potential to use weak monoclonal antibodies of IgG and IgM for affinity chromatography has recently been examined. Strandh, M., et al (1998)” New approach to steroid separation based on a low affinity IgM antibody” J. Immunol. Methods 214, 73-79. Using the smaller binding motifs such as antigen binding site of antibodies and the recent developments in antibody and other protein engineering. Molecular cloning techniques have recently been developed to generate repertoires of antibody derived binding sites (Hayden, M. S., Gilliland, L. K. and Ledbetter, J A (1997) “Antibody Engineering” Curr Opin Immunol 9, 201-212; Smith, G. and Petrenko, V (1997) “Phage display” Chem Rev 97, 391-410.

Direct attachment is possible, but use of spacer arms (e.g. hexamethylene) often provides good adsorption during affinity chromatography. The same can apply to biospecific desorption. Surprisingly, the hydrophobic interactions of the spacer arm can provide a helpful part of the binding to the adsorbent. The energy of interaction ΔG°=−RT ln K_{d} is made up of the specific interaction ΔG° (specific) between the binding pair and nonspecific interactions ΔGo (nonspecific). Therefore the energy of interaction is ΔG° (interaction)=ΔG°(specific)+ΔG°(nonspecific).

For example, if K_{d} (specific)=0.002 mM and K_{d} (nonspecific)=0.1 mM, then

ΔG° (interaction)=ΔG°(specific)+ΔG° (nonspecific)

ΔG° (specific)=21 kJ/mol and ΔG°(nonspecific)=11.5 kJ/mol, hence the ΔG° (interaction)=32.5 kJ/mol.

Suppose now that a free ligand is contacted with the immobilized receptor and this completely displaces all specific interactions as the ligand binds to its cognate receptor. Then the ΔG° (specific) becomes zero and only the nonspecific forces remain. Since these amount to only 11.5 kJ/mol which is too low to cause any significant retention in the flow passage, the ligand is specifically desorbed. A total energy of interaction between a protein and matrix of about 30 kJ/mole is needed to retain a protein-binding pair a flow passage. This interaction energy is often not available by a single protein-ligand interaction. It can be reasoned that even weak nonspecific interactions are sufficient to add to the specific ones to create quite strong binding overall. Surprisingly nonspecific interactions are purposely introduced in some embodiments of the subject invention. Typically one would expect that nonspecific interactions should be avoided. Single weak binding may also be strengthened by multiple point binding of the immobilized binder to the matrix or by immobilizing multiple binders to the same molecule (e.g., a dextran strand or polypeptide). High density charge groups such as DEAE—for negatively charged binders may be introduced in spacers or matrixes. Flexible polymers having branched structures or small ligands (e.g., antibody binding domains rather than entire antibodies or protein binding motifs, domains, fragments) immobilized at high concentrations using spacers and site specific binding with the ligand binding site orientated away from the surface and freely available to bind its ligand can facilitate binding of weak binders.

The microsystems provided herein can allow for very rapid trial-and-error optimization of binders and buffer conditions specific for each particular biospecific desorption event. The current art employs trials of the effectiveness of the adsorbent in a Pasteur pipette or a 1 or 2 ml column; a sample of the binder in a suitable buffer is applied and the column is washed. If the desired binder sticks under these conditions one can assume that adsorption has been achieved. This method is slow, laborious and costly. Furthermore, it consumes large amounts of sample. Although biospecific desorption by inclusion of the free ligand in the buffer is the ideal method to elute an analyte in affinity chromatography, it is not commonly used. The reason is that the ligand is costly. Large amounts of ligand are needed. In the present invention only tiny amounts of free ligands are used to biospecifically desorb the ligands. In the current invention this process is automated and instead of using milliliters of reagents and sample microliters to subnanoliter volumes are employed.

The buffer used is important to the binding. Many affinity ligands are charged. At low ionic strength these can act as weak ion exchangers. To avoid binding unwanted proteins the ionic strength is typically reasonably high (e.g., a binding buffer may contain 150 mM NaCl). The buffer conditions can depend on the specific binding pair or complex under study. Biospecific interactions may be weakened or strengthened by higher ionic strength or other buffer conditions. Thus, different buffers can be used to weaken strong biospecific interactions and other buffers can be used to strengthen weak interactions. Salt concentration, pH, and temperature may be varied to promote or diminish hydrophobic interactions, ionic interactions, or hydrogen bonding. Since binding depends on the concentration of binders and the microenvironment, the binding constant is not restrictive as is commonly thought by those in the current art.

These methods and systems can allow rapid trials for biospecific desorption to be carried out using tiny amounts of sample and reagent in an automated microsystem with computer controlled fluidics and detection.

The principles of biospecific desorption as disclosed herein apply to affinity adsorbents, ion exchangers, or any other adsorbent. If the buffer is changed to reduce the apparent binding constant, a much lower concentration of ligand can be employed for desorption. Increasing the salt minimizes ionic interactions but also increases hydrophobic interactions. Introduction of surface tension-reducing agents can lessen hydrophobic interactions. Surface tension reducing agents include, for example, nonionic detergents such as Triton X-100, ethylene glycol, and isopropanol.

The procedure can work as follows. After a prewash with the optimized buffer, the free binder is introduced to the binding pair or complex in the same buffer. Biospecific desorption is achieved even as nonspecific forces are introduced into the system to allow biospecific interactions to be studied. In practical terms the mechanism by which biospecific desorption operates in embodiments employing immobilized binders is clear. The term biospecific desorption does not require any particular biospecific property of the adsorbents itself. The concentration of ligand needed for the biospecific desorption depends on the buffer conditions, temperature, and binding constant.

Biospecific desorption from an ion exchanger may be employed in some embodiments of the current invention. For example, a binder is adsorbed at a certain pH because the electrostatic interactions between the matrix of the adsorbent and the charges on the protein are strong enough to hold it. If the free ligand bound receptor is contacted with the adsorbed receptor and it is charged and of opposite sign to the net charge on the adsorbed binding partner then the bound complex has a decreased net charge on the bound complex. This causes specific elution of the binding complex and no other adsorbed binder. In this aspect of the invention multiple unique binders may be adsorbed in the same flow passage and different cognate binders flowed through the chamber. Binders immobilized in the flow channels may bear uniquely distinguishable labels such as those known in the current art. This can facilitate the simultaneous biospecific desorption and detection of multiple binding pairs simultaneously.

The use of frontal affinity chromatography for the estimation of binding constants and the binding capacity for various compounds is convenient and reliable provided that the binding-site population is not heterogeneous in nature. This procedure involves saturation of the column by the free binder (which may be labeled) at various concentrations, which renders chromatograms describing the elution profiles each comprising an elution profile and a front.

The elution volume (V) depends on the concentration of free binder flowed through the microflow passage and the affinity between the analyte and immobilized binder and may be determined by the inflection point in the front. Vo describes the front volume when no biospecific desorption occurs. By plotting 1/((L*) (V−Vo)) vs 1/(L*),−Ka (the association constant) can be calculated from the intercept on the abscissa. The intercept on the ordinate reflects 1/Qmax. (L*) is the concentration of free labeled ligand.

A reference system is important to biospecific desorption assays and controls are run in parallel with the systems. The control is preferably identical to the “real biospecific binding flow passage” except it will not contain the a cognate biospecific binding partner.

In some embodiments of the current invention, nonspecific interactions are purposely introduced by using hydrophobic and/or charged matrix to enhance immobilization of a binder. In certain embodiments these nonspecific binding helper molecules can be bound or conjugated to one or more member(s) of a binding pair or complex of multiple binders in solution. This can facilitate specific binding and may also increase the sensitivity of detection. For example, matrix assisted adsorption can add to the mass of attached or adsorbed binders increasing the signal of detection in some embodiments (e.g., fluorescent polarization, diffusion based detection methods). Biospecific desorption of a matrix assisted binding pair can lead to decreased diffusion times. Labels (e.g., fluorescent labels, or optical particles) including multiple labels for different binding pair members (e.g., different colors of fluorescent dyes, or different size beads or particles (e.g., nanoparticles). The diffusion current is roughly proportional to the length of the adsorber not to the area of the adsorber. Given the surprising high rate at which particles adsorb to thread-like objects, preferred matrix materials are thread-like molecules such as dextran, heparin, hyaluronic acid, oligopeptides, nucleic acids, polypeptides and thread-like proteins such as those composed of coiled coils (e.g., collagens or parts of proteins including engineered or synthetic proteins). As stated above, in some embodiments of the current invention nonspecific interactions are purposely introduced by using various hydrophobic and/or charged thread-like matrices. These may be engineered as branched structures composed of various amino acids. Hydrophobic as well as charged amino acid residues are used as well as branched structures. These structures may be comprised of the D-isoform of the 20 natural amino acids with possibly lys and cys residues introduced as cross-linking sites for the conjugation of other peptides. Biomolecules may be attached to these “branched peptide trees” using lys and cys residues and commercially available chemical cross-linking reagents to provide multiple attachment sites for ligands on the same molecule. For example, maleimidobenzoyl-N-hydroxysuccinimide ester (MBS) is a useful reagent for attaching peptides by way of cysteine residues (cysteine sulfhydryls to amino groups); water-soluble carbodiimides may be used to attach carboxyl- to amino-residues, and glutaraldehyde may be used to attach amino- to amino-. Other suitable matrix materials include polysaccharides, modified polysaccharides, silica, polystyrene and agarose sepharose.

In biospecific desorption and particularly competitive displacement assays, immobilized binders can be preloaded with labeled (e.g. fluorescently labeled) binding partners. Molecular recognition or biospecific interactions of the binders is achieved when the surfaces of the binders match well enough to form enough weak bonds to withstand thermal motion. This specific binding is not fixed or permanent; it is governed by a dynamic equilibrium, in which molecules are continually being bound and released. And at any instant the percentage of bound molecules depends on the relative amounts of the binders present and the strength of the association between them.

With respect to biospecific desorption of the competitive kind, and without being wed to theory, when the binder of interest is flowed through the microchannel bearing the binding complex, it competes with the labeled binder for binding to the immobilized binder, as a result, the total amount of labeled binder bound to the immobilized binder decreases. The “displaced” labeled binder is released and can be detected. In another variant of biospecific desorption, the binding of an allosteric modulator of the binding pair interaction can be studied.

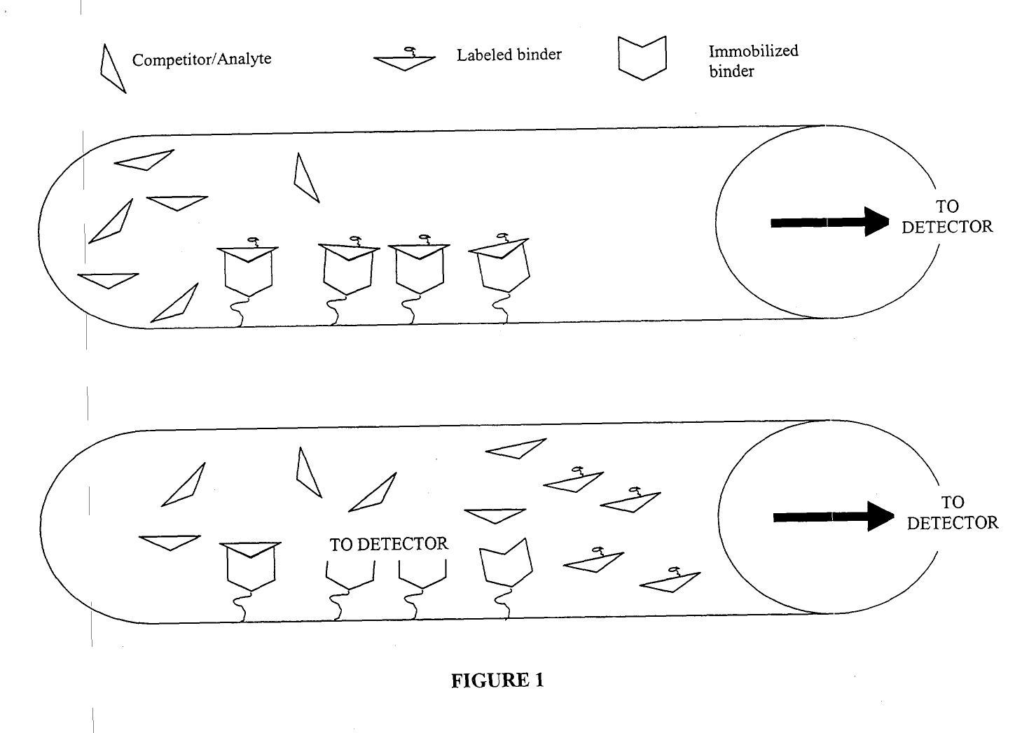

FIG. 1 illustrates a biospecific desorption having an immobilized prebound complex of a first immobilized binding member bound to a labeled second binding member. Upon contacting the prebound complex with a sample containing an analyte capable of binding the immobilized member, the labeled binder is freed from its binding pair. The assay is based upon detecting the desorption of the binder. For instance, the desorption could be immediately detected as a change in the fluorescent polarization of the label on the desorbed binder. In the embodiment of FIG. 1, the released labeled binding member is released into the fluid medium and carried thereby to a downstream detector. The precise mechanism of displacement is not crucial to the operability of the systems and methods of the invention. It does not matter, for instance, whether the labeled binder first dissociates from the immobilized binder and then the free ligand/analayte binds to the immobilized binder or whether the free ligand/analyte actively pushes the labeled binder off of the immobilized binder or otherwise interferes with the binding interactions of the binding pair members.

For biospecific desorption in the current invention a major determinant of the response time for a biospecific desorption event is the effective dissociation constant of the binding pair members. A relatively large effective dissociation constant is useful for rapid response. For example, effective dissociation rate constants may be in the range of 0.001-0.00001/sec.

The microflow passage is preferably saturated or substantially saturated with prebound binder.

When antibodies are employed as binders, for example, antibodies recognizing post translational modifications on proteins,(i.e., anti-tyrosine phosphate, anti-serine phosphate, anti-nitrotyrosine, and anti-carbohydrate antibodies), the antibodies preferably have relatively large effective dissociation rate constants. The thermodynamic description of binding make no reference to the speed at which the association or dissociation occurs. For biospecific desorption experiments the kinetic parameters are important. The dissociation rate constant, k_{diss} gives the speed at which members of a complex dissociates. The dissociation rate constant is dependent on the concentration of the complex. Its units are those of reciprocal time. For example, a dissociation rate constant of 0.0001/sec means that one in 10000 of the of the binding pair complexs present comes apart each second. And the association rate constant gives the speed at which the binders associate to form the complex. For the complex AB, its speed depends on the concentrations of both A and B. At equilibrium, by definition the thermodynamic dissociation constant K_{d}=k_{diss}/k_{assoc}.

In some embodiments of the invention, the complex may need to stay associated during some series of steps. In other cases an analog of a member of the binding pair (dummy binder) which forms a stable complex with an immobilized binding partner can be bound to the immobilized binder and displaced by a high concentration of the real cognate binding partner. Rates depend on the buffer conditions, temperature, concentration and can be optimized for use in system. Biosensor technology provides sensitive methods for the rapid determination of rate constants. Analysis can be accomplished according to the methods and systems of the invention over a wide range of binding pair affinities as well as size of both immobilized and mobile binders. This analysis can be performed on a microscale dependent only on the limits of detectability of the binder eluting from the affinity column. The dissociation constant of the binder pair is a factor in the operability of the invention. Means have been developed for weakening binding where it is too strong and for strengthening binding when it is too weak.

Surprisingly, in some embodiments the method can even be adapted to work with binding member pairs whose affinity constants are greater than 1 micromolar, 10 micromolar, 100 micromolar, and even 1000 micromolar. The method can be preferably adapted to work for binding pair members whose affinity constants fall within the range of 10 micromolar to 100 micromolar, 50 micromolar to 500 micromolar; and 250 micromolar to 1000 micromolar or greater.

Methods for determining the dissociation constants of antibodies and other ligands are well known in the prior art. Antibodies or fragments thereof or oligonucleotide aptamers that specifically bind any known co- or post-translational modification on proteins can be obtained having the desired dissociation constants using known methods. The systems invented herein are suitable for rapidly identifying co- and post-translational modifications on proteins. Specific examples and preferred embodiments are set forth below.

The microflow analytical devices and methods of the present invention can be used to perform specific microflow competitive displacement assays in which the displacement of one prebound member of a binding pair from the complementary binding pair member is used to detect the ability of a sample to modify or compete with or inhibit the interaction of the biomolecular recognition binding pair members. Typically, the system operates by providing a first binding pair member(s) immobilized within a chamber or channel to be exposed to a flow stream or immobilized to the surface of a channel for conducting the flow stream and a second binding pair member reversibly bound to the first binding pair member. The immobilized binding pair member(s) may be in direct or indirect contact with the microflow flow stream. An example of an indirect contact with the fluid flow is where the immobilized binding pair is separated from the fluid flow by a permeable membrane that allows the analyte(s) of interest to penetrate the membrane and thereafter contact the immobilized binding pair member.

Microflow specific desorption analysis measures interactions between two or more molecules by monitoring the desorption of the prebound binding member caused by the free binder or an analog of the desorbed binder. Any binding assay known in the art may be used to monitor this desorption event without departing from the scope of this invention. The methods can be applied to all molecules expressing affinity for each other such as biomolecules (proteins, nucleic acids, carbohydrates, lipids), low molecular weight compounds (signaling substances, pharmaceuticals, vitamins, pesticides, pollutants, etc).

The desorption event may be monitored in a number of ways. For example, one of the interactants may be immobilized in a microflow channel and then a labeled binder may be bound to the immobilized binder. A solution containing the analyte is automatically passed over the surface under controlled flow conditions. The analyte may cause a desorption of the labeled binder which may then be detected. Using some detection schemes a label may not be necessary. For example, if surface plasmon resonance or piezoelectric crystal based biosensors are used as transducers, a mass change upon the desorption of the bound molecule or substance (e.g. cell, virus, phage) may be allow the desorption event to be quantitatively monitored in real time without a label. In other embodiments it may not be necessary to immobilize the binders.

The desorption event may be monitored in continuous flow for example by using fluorescent techniques such as fluorescent polarization, fluorescent energy transfer, or fluorescence correlation spectroscopy. Using these techniques the change in fluorescence is continuously monitored as the biospecific desorption event takes place. For example, fluorescence correlation spectroscopy and fluorescence polarization are ultrasensitive and can be used in continuous flow to monitor binding or desorption in real time. Fluorescence correlation spectroscopy allows binding to be determined in biological assays at the single molecule level. Homogeneous assays are compatible with microtiter plates; however, Microsystems containing a multitude of sub-microliter sample wells or channels may be used in combination with a nanopipetting and sample retrieval system and/or microflows systems.

Strong binders may be weakened and the binding of weak binders may be strengthened thereby optimizing conditions for a successful and rapid biospecific desorption.

Another way to increase the dissociation constant of the labeled binder is by the conjugation of a label. The introduction of a fluorescent label into a binder may often introduce steric hindrance or other factors which result in a weaker binding to its ligand or receptor compared to the unlabeled binder. This may mean that the dissociation constant is smaller for the unlabeled binder and may facilitate the desorption or biospecific elution of the labeled binder by its unlabeled analog.

The micro flow systems invented herein may enable the rapid change in buffers to obtain those suitable for optimizing the systems for biospecific desorption.

For good adsorption the dissociation constant must be less than approximately 10⁻⁵ M, smaller than most protein-ligand dissociation constants. For biospecific interactions involving dissociation constants larger than about 10⁻⁵ M additional binding energy may be obtained by the selection of a suitable matrix for immobilization of the binder. Direct attachment may not be satisfactory, but the use of a spacer arms, for example, hexamethylene, may give good adsorption of labeled binder.

Analysis of the apparent dissociation constant can be conducted under various buffer conditions, at various temperatures, and at various flow rates and adsorbent concentrations. The term apparent dissociation constant reflects the fact that the constant is calculated from the amount of labeled binder analog released from the column. This constant is a function not only of the actual dissociation constant of the binding pair but also of independent factors, such as the nonspecific binding of labeled and unlabeled analyte molecules and the accessibility of the binding site.

The affinity constants of binders depend on the microenvironment. The microenvironment typically employed for binding studies has no relationship to the microenvironment experienced with the binding partners in their native microenvironment. The microenvironments in cells, blood, and extracellular matrix is dynamic and extremely crowded. Diffusion times within cells or mass transport times due to metabolic channeling are short due the short distances molecules and ions must travel. Most laboratory studies, for example, use dilute aqueous solutions of enzymes. In such studies, it is common for the substrate to be present at 1000000 times that of the enzyme whereas in fact the concentrations of enzymes, substrates and modulators in the cell are comparable for major metabolic pathways. Model analytical systems usually take little notice of the influence of concentration effects on the interactions between metabolites and macromolecules. There is a clear need for scientists to examine the concepts of molecular recognition and biospecific adsorption and desorption in the light of realities of biological microenvironments. Commonly neglected in in vitro studies of metabolic control is the concentration. The high concentration of certain proteins in the cell are known to influence the localization of free metabolites; they may exert potent effects on metabolism or pathology via such concentrations rather than by virtue of other biological mechanism. However, these proteins are costly and their use using current art methods is cost prohibitive. However, the microflow systems invented herein can use tiny amounts of samples providing for studies which mimic the actual conditions encountered within cells, extracellular matrices and fluids. Another consideration is diffusion times. Molecules must collide in order to react. Diffusion is a fundamental process in the movement of materials. Those diffusion processes that are of biological importance take place over short distances, a fraction of a millimeter. Over longer distances transport must take place by mass movement (e.g., flow). Diffusion of biological importance is limited to short distances because diffusion time increases with the square of the diffusion distance. Consider the time it takes for substances to travel a given distance by diffusion, microchannels and microflow systems are preferred means of incorporating such dynamic factors into a study of binder interactions and biosystem behavior on a microscale substantially closer to that of the cell.

TABLE 1
DIFFUSION TIMES TN WATER AT 37° C.

| | Diffusion coefficient | | | Time taken to diffuse | | | |
|---|---|---|---|---|---|---|---|
| | (cm2/sec) | 1 micrometer | | 10 micrometer | | 1 millimeter | |
| small | 5 × 10⁻⁶ | 1 | msec | 0.1 | sec | 17 | min |
| molecules | | | | | | | |
| protein | 5 × 10⁻⁷ | 10 | msec | 1 | sec | 2.8 | hr |
| molecule | | | | | | | |
| virus | 5 × 10⁻⁸ | 0.1 | sec | 10 | sec | 28 | hr |
| particle | | | | | | | |
| bacterium | 5 × 10⁻¹⁰ | 1 | sec | 100 | sec | 12 | day |
| animal cell | 5 × 10⁻¹⁰ | 10 | sec | 17 | min | 117 | day |

Table 1 is provides estimates of diffusion times for biological entities of interest to the current invention. These times are approximate values and the diffusion time will depend on the size and shape of the particle as well as temperature and viscosity of the fluid in which the substance exist. The approximate values in the table give a rough indication of the diffusion constants of a given type of particle of increasing size and are not exact.

From the above table, one can see the advantages of using channels of submillimeter dimensions to increase reaction rates by decreasing the diffusion distances between binding partners.

Samples

A “sample” is a medium containing a substance of interest, synthetic or natural, to be examined, treated, determined or otherwise processed to determine the amount or effect of a known or unknown analyte therein. “Analyte” refers to the constituent of a sample to be detected or quantitated by the desorption of a labeled binder from its binding partner. Typical sources for biological samples include, but are not limited to, body fluids such as, for example, whole blood, blood fractions such as serum and plasma, synovial fluid, cerebrospinal fluid, amniotic fluid, semen, cervical mucus, sputum, saliva, gingival fluid, urine, and the like. In addition, sample includes combinatorial chemistry generated libraries of compounds, usually small molecules, oligonucleotides and peptides. Other sources of samples are aqueous or water soluble solutions of natural or synthetic compounds, particularly, compounds that are potential therapeutic drugs where it is desired to determine if the compound binds to a specific receptor. The sample can be a biological sample including fermentation broth, proteolytic digest or cell culture medium. Environmental, pharmaceutical, air, and food-derived compositions are also within the scope of “sample”.

The amount of the sample depends on the nature of the sample and the nature of the processing to be conducted. For fluid samples such as whole blood, saliva, urine and the like the amount of the sample is usually about 1 to 1000 nanoliters, more usually, about 10 to 100 nanoliters. The sample can be pretreated and can be prepared in any convenient medium, which does not interfere with a microflow process in accordance with the present invention. An aqueous medium is preferred. The term “sample” refers to a composition whose effect on the biomolecular interaction is to be studied. Samples may be synthetic, isolated, impure, partially purified, or otherwise a complex mixture. Samples can be delivered in fluid form as a solution or mixture.

The use of controls and standard curves in determining the concentration of an analyte in a sample are well known fundamentals in the art. For instance, the concentrations of an analyte in a sample may be determined by measuring the desorption of a prebound binding member from its immobilized partner and comparing the amount desorbed or desorption time course value with values obtained in the same way using one or more standard samples of known analytes and known concentrations. A preferred embodiment provides a standard curve for each of the analytes to be analyzed. In another preferred embodiment, a microprocessor receives the detection signal and thereby analyzes the data according to a standard curve to provide the amount.

Binders

The terms “binder” or “binding member” are used herein to refer to a molecule or substance or cell that preferentially and non-covalently binds another molecule or substance or cell. Preferred binders may be any biomolecule or fragment thereof, including drugs, and toxins. A “biomolecule” is a biologically active molecule. Examples of binders include, but are not limited to, proteins (especially antibodies and receptors) and fragments thereof, carbohydrates, drugs, metals, cofactors, lipids, metals, metal chelators, peptides, polynucleotides, nucleotides, peptide nucleic acids, polynucleotides, hormones, inhibitors, dyes, amino acids, polysaccharides, part of a RNA or DNA molecule, part of a peptide or polypeptide corresponding to a motif or domain in a protein, a carbohydrate corresponding to a glycoprotein, a lipid corresponding to a lipoprotein, fragments of any biopolymer, aminoacyl-tRNA synthetases, tRNAs, elongation factors, antibodies, antibody fragments, aptamers, and ribosomes that possess binding activity.

Binding pair members or partners comprise different molecules each having a portion thereof that interacts with a particular portion of the other member of the binding pair. The binding pair members therefore possess complementary spatial arrangement of polar and other surface properties which provide a preferential binding. The members may be a ligand and its receptor or an antibody and its antigen. Binding pair members can be small molecules or residues of small molecules and their receptors or can be large molecules such as proteins and other biopolymers. Binders can be cells or their constituents.

As with antibodies, oligonucleotides or peptide aptamers that specifically recognize an analyte can be produced using known methods. Aptamers are a particularly attractive class of binders. Aptamers can now be provided which can recognize virtually any class of target molecule with a high affinity. (See Jayasena S D (1999) Clin Chem 45:1628-50; Kusser W. (2000) J. Biotechnol. 74: 27-39; Colas P. (2000) Curr Opin Chem Biol 4:54-9) Aptamers which specifically bind arginine and AMP have been described as well (see Patel D J and Suri A K, (2000) J. Biotech. 74:39-60.

A ligand is a binder for which a receptor naturally exists or can be prepared.

A receptor is any compound or composition capable of recognizing a particular spatial and polar organization of a molecule, e.g., epitopic or determinant site and thereby binding to the molecule. Illustrative receptors include membrane bound receptors such as G-protein receptors (e.g., muscarinic, adrenergic, prostaglandin and dopamine such as the D2 receptor), tyrosine kinase (insulin-like IGF, epidermal EGF, nerve NGF, fibroblast FGF growth factors), ion channels, T-cell receptors, the interleukins, and other naturally occurring receptors, e.g., thyroxine binding globulin, antibodies, enzymes, Fab fragments, lectins, nucleic acids, protein A, complement component Clq, and the like.

Two important groups of proteins for use as binders are the serine proteinases and the standard mechanism, canonical protein inhibitors of serine proteinases. These proteins termed “serpins” are found widely throughout nature. They are found, for example, in plants, animals, insects, and certain viruses. Proteinases are ubiquitous to life; they turn many processes on and off, but they are dangerous and must be tightly controlled. Proteinases and their inhibitors play very important roles in human physiology and diseases. Proteinases and their inhibitors are involved in blood coagulation, wound healing, cell migration, immunology, developmental biology, and protein hormone action. Proteinase inhibitors are important therapeutic agents in the fight of diseases including AIDs, blood coagulation disorders, neurodegenerative diseases and others. The average number of protons released per mole of complex formed by standard mechanism serine proteinase inhibitors is large and positive.

The quantitative description of this system has been described by Lebowitz, J and Laskowski, M. Jr. (1962) “Potentiometric measurement of protein-protein association constants. Soybean trypsin inhibitor-trypson association” Biochemistry, 1, 1044-55 and later by Tanford (Tanford, C (1968) Protein denaturation. In Advances in Protein Chemistry, (eds C. B. Anfinsen, Jr., M. L. Anson, J. T. Edsall and F. M. Richards)pp. 122-282. Academic Press, New York. Generally, the following formulae apply:

E+I⇄C+qH+ (1)

E=enzyme (proteinase); I=inhibitor (serine proteinase inhibitor); C=complex of E and I; q=the average number of protons released

dlogKₐ/d(pH)=−q (2)

$$log Ka ( {pH}_{2} ) = log {K}_{a} {pH}_{1} + {\int }_{{pH}_{1}}^{{pH}_{2}} q ⅆ ( pH ) \qquad ( 3 )$$

From the above equations it follows that equation 3 can be used to measure very large KₐpH₂, by using the much smaller and easier to measure KₐpH₁. All that is needed is the average value of protons released upon complex formation as a function of pH over pH₁ to pH₂ range.

“Immobilized binder” refers to a binder that is non-covalently or covalently localized as by attachment to a surface including surfaces of cells, proteins to a matrix which may be a synthetic or biopolymer or an extracellular matrix created in a flow chamber, e.g. nanoparticle, phage, cell, polymer, tissue or other biological or nonbiological material. An immobilized binder can be applied to the surface by a vast number of methods known in the arts. Multiple binders are used in some embodiments. For example cells may be immobilized to the surface of a channel and then The method of immobilization or attachment is not critical to the present invention as long as the immobilized binder retains its ability to bind its ligand and is not transported away by the flow stream.

Typically the immobilized binder is selected to bind the analyte and analyte analog or a complex thereof. The immobilized binder may be chosen to directly bind the analyte or indirectly bind the analyte by binding to a binder that is bound to the analyte.

The immobilized binder(s) may be configured as single or multiple capture sites. The immobilized binders may be presented in a variety of configurations to produce different detection formats. Alternatively the immobilized binder may be distributed over a large portion of the flow channel. The extent of signal production generated in the capture site is related to the amount of analyte that can displace the analyte analog and hence to the amount of analyte in the test sample.

In some embodiments of the current invention, a binding pair member is immobilized to surfaces (e.g., beads, microspheres, the bottoms of microwells, microchannels, optical fibers or other biosensor transducers). The members may be immobilized by covalent or noncovalent attachment. These molecules may be immobilized, for example, using chemical cross-linkers to covalently attach them to a surface, by adsorption, entrapment, encapsulation, or by binding to a protein, nucleic acid, or peptide nucleic acid. For example, the binding pair members may be immobilized by electrostatic binding to molecules such as poly-L-lysine. Furthermore, binding pair members may optionally be cross-linked to a suitable spacer arm and attached to a solid support. Biotinylated tRNAs, for instance, may be immobilized by binding to avidin or streptavidin. The chemical modification can encompass several strategies. The initial Derivatization may be to add a spacer arm to a particular reactive group. The spacer may optionally contain a terminal functional group that can be used to couple to another molecule or to a surface. Chemical modification, cross-linking, and immobilization of nucleic acids are taught in a number of references. For example, see, Hermanson (ed) (1996) Bioconjugation Techniques pp. 639-671. The spacer arm is preferably long enough to eliminate most steric hindrance caused by the solid surface to ensure the efficiency of the biomolecular interaction. Additionally, the spacer arm should permit no unwanted nonspecific binding. For example, using nucleic acids as spacers, Shchepinov et al. (1997) Nucleic Acids Research. 25: 1155-1161, have demonstrated that an optimal spacer length is at least 40 atoms long and can increase the hybridization yields of nucleic acids by 150 fold.

Immobilized binders such as proteins, peptides, protein fragments, nucleic acids, lipids, carbohydrates, vitamins, drugs or substances (beads, particles, metals, cells, virions, viruses of organelles, membranes vesicles, organelles and other substances) can be covalently or non-covalently attached onto the surface of the structures or within the capillaries or microchannels. A vast number of techniques for placing immobilized reagents for binders (e.g. proteins, cells, viruses, phages, carbohydrates, drug, nucleic acids, carbohydrates, lipids and the like) on surfaces are known to those skilled in the art.

The main requirements for a successful affinity adsorbent are: the binder be attached to the matrix in such a way that that the binder's affinity for the binding partner concerned is not substantially disturbed; a spacer arm setting the binder away from the matrix can be used to make it more accessible to its binding partner; and the linkages should be stable to the conditions of use.

It is now possible to obtain in nanoparticle size a variety of particles make from ceramics, metal oxides, plastics, glasses, proteins, carbohydrates, the like. These particles, which may be derivatized, may be reacted with proteins, lipoproteins, glycoproteins, drugs, haptens, oligonucleotides, cells, viruses and the like. With nanoparticles the activities of the various biological molecules attached thereto is normally retained as taught in U.S. Pat. Nos. 5,219,577 and 5,429,824.

Many specific chemistries have been developed for the attachment of ligands to surfaces. Methods for immobilizing proteins, carbohydrates, lipids, cells, viruses, nucleic acids, and small molecules are taught in the following references, and others (O'Neill, C., et al (1986) Cell. 44: 489; Kleinfeld, D., et al (1988) J. Neurosci. 8:4098; Clark, P (1996) In Nanofabrication and Biosystems (ed. H. C. Hoch, L. W. Jelinski, and H. G. Craighead), p. 356. Cambridge University Press, New York; Singhvi, R et al., (1994) Science, 264, 696; Saleemuddin, M (1999) Adv Biochem Eng Biotechnol. 64: 203-26; Turkov, J (1978) Affinity Chromatography. Elsevier Scientific, Amsterdam; Mohr, P and Pommerening, K (1985) Affinity Chromatography: Practical and Theoretical Aspects, Dekker, NY; Ostrove, S (1990) Affinity Chromatography: General Methods Methods Enzymol 182, 357-371; Mosbach (1976) Meth. Enzymol. 44: 2015-2030; Hermanson, G. T. (1996) Bioconjugate Techniques, Academic Press, N.Y.; Bickerstaff, G. (ed) (1997) Immobilization of Enzymes and Cells, Humana Press, NJ; Cass and Ligler (eds) Immobilized Biomolecules in Analysis, Oxford University Press; Watson et al. (1990) Curr. Opin. Biotech. 609:614; Ekins, R. P. (1998) Clin. Chem. 44: 2105-2030; Roda et al. (2000) Biotechniques 28: 492-496; Schena et al. (1998) Trends in Biotechnol. 16: 301-306. U.S. Pat. No. 5,700,637 {Southern, 1997); U.S. Pat. No. 5,736,330 (Fulton, 1998); U.S. Pat. No. 5,770,151 (Roach and Jonston); U.S. Pat. No. 5,474,796 (Brenman, 1995) all of which are incorporated by reference herein).

Any system of binder attachment capable of orienting the molecules on the test surface so that they may have maximum activity is generally preferred. The receptor (binder) molecule can be attached to the surface by adsorption, gel entrapment, covalent binding or other similar methods. Covalent binding is preferred. Preferably, the linkers orientate the recognition molecules in such a way as to favor complex formation such as the linking entity used in Newman U.S. Pat. No. 4,822,566.

Many coupling agents are known in the art and can be used to immobilize binders in the methods and devices of the present invention. Coupling agents are exemplified by bifunctional crosslinking reagents, i.e., those which contain two reactive groups which may be separated or tethered by a spacer. These reactive ends can be of any of a number of functionalities including, without limitation, amino reactive ends such as N-hydroxysuccinamide, active esters, imidoesters, aldehydes, epoxides, sulfonyl halides, isocyanate, isothiocyanate, nitroaryl halides, and thiol reactive ends such as pyridyl disulfide, maleimides, thiophthalimides and active halogens.

As described in U.S. Pat. No. 4,824,529, hydroxyl functional groups are commonly introduced to the surfaces of glasses, semiconductors, metal oxides, metals and polymers. These hydroxyl groups react with commercially available linkers such as (3-aminopropyl) triethyloxysilane or with thiol-terminal silanes, for example. To these amino or thiol-terminal silanes one may then graft the desired peptide, protein, lipidic, or glycosidic moiety via homobifunctional crosslinkers such as gluteraldehyde or via heterobifunctional crosslinkers.

Cross-linking reagents may find use in the subject invention in immobilizing binders (e.g., biomolecules, cells, viruses and the like) and in the conjugation of labels such as fluorescent labels to binders. Commercially available heterobifunctional crosslinkers for use in the present invention include, but are not limited to, the maleimido-NHS active esteers, such as succinimidyl 4-(N-maleimido-methyl)cyclohexane-1-carboxlate (SMCC); m-maleimidobenzoyl-N-hydroxy-succinimide ester (MSB); succinimidyl 4-(p-maleimidophenyl)butyrate (SNPB); N-succinimidyl 3-(2-pyridyldithio) propionate (SPDP); N-succinimidyl m-maleimidobenzoate (Sulfo-SMB); and N-succinimidyl-3(2-pyridyldithio) propionate (SPDE) (Pierce, Rockford Ill). This list is not intended to be exhaustive. Over 300 cross-linkers are currently available for the conjugation of biomolecules (reviewed in Wong, S. S. (1993) In Chemistry of protein conjugation and crosslinking, CRC Press, Boca Raton).

Various materials may find use as solid phases for the immobilization of binders in the subject invention. These include chromatographic media or materials that are well known to those skilled in the arts. Such materials include: ion-exchange materials such as anion (e.g. DEAE) and cation exchange, agarose, hydrophobic interaction materials, affinity chromatographic materials having a binding member covalently bound to the insoluble matrix via a spacer arm, where the specific binder may be a lectin, drug, cofactor, inhibitor, protein A, antibody, antibody fragment, oligonucleotide, aptamer, protein fragment, nucleotide, metal, dye and the like. The insoluble matrix to which the binding member is bound may be particles, such as polymeric beads, porous glass, magnetic beads, nanoparticles, networks of glass filaments or microstructures, multiple narrow rods or the wall of the microchannel or capillary and the like. A retention means may be employed as needed to keep the chromatographic material in the reaction channel. Glass frits may be used to cover the fluid inlets and outlets of the reaction channels. Such frits, where employed, may allow the macromolecules, and other samples including cells to flow through the channels but may retain the solid phases.

Conventional methods for protein and nucleic acid immobilization may be used for binder immobilization. Proteins and nucleic acids have been immobilized in a vast number of ways over the last 30 years and many references can be found describing various immobilization techniques. Proteins and nucleic acids have been immobilized on biosensors, microarrays, microspheres, nanoparticles, and a multitude of other supports. Adsorption, entrapment, encapsulation, cross-linking and covalent attachment are among the techniques employed for immobilization of biomolecules. Proteins and nucleic acids may be encapsulated by enveloping the molecules in various forms of semipermeable membranes, entrapped in gel lattices, adsorbed onto or covalently attached to surfaces. For example, proteins and nucleic acids may be entrapped in gels along with fluorescent or other indicators (Flora and Brennan (1999) Analyst 124:1455-1462). These biomolecules may be encapsulated into sol-gel derived materials prepared either as monoliths or beads. A support-free type of immobilization is crosslinking. This method involves joining of proteins to each other to form three-dimensional complex structures. Chemical methods for crosslinking normally involve covalent bond formation between the proteins by means of a bi-or multi-functional reagent, such as glutaraldehyde. Strategies for reversible immobilization of proteins include reversible chemical interactions (Tyagi, et al.(1994) Biotechnol. Appl. Biochem. 20:93-99) in particular metal chelation (Gritsch et al.(1995) Biosens. Bioelectron. 10: 805-812) or disulfide cleavage (Batistaviera et al.(1991) Appl. Biochem. Biotech. 31: 175-195), protein-ligand interactions (Phelps et al. (1995) Biotechnol. Bioeng. 46, 514-524) and nucleic acid hybridization (Niemeyer et al. (1994) Nucleic Acids Res. 22: 5530-5539).

Methods for site-selective immobilization of biomolecules applicable to binders have been developed. This can facilitate the fabrication of spatially defined ligand-receptor arrays for biosensors and parallel-ligand binding assays on microarrays. For example, immobilization of immunoglobulins was achieved by photolithography techniques (Rozsnyai, et al. (1992) Angew Chem. Int. Ed. Engl 31, 759).

Nucleic acid-directed immobilization of proteins provides a single site-selective process for the immobilization of proteins and other biomolecules under mild chemical conditions (Niemeyer et al.(1998) Anal. Biochem. 268, 54-63). Oligonucleotide arrays are widely used for DNA analysis (e.g., Kozal et al. (1996) Nat. Med. 2: 753-759) and such arrays are used as standard array templates for the constructing of arrays of any biomolecule that can be attached to a single stranded nucleic acid. The single stranded nucleic acid is then hybridized to its complimentary strand immobilized in a known location on a surface. This method of arraying protein and nucleic acid binders may be employed in some embodiments of the subject invention.

Other methods for immobilizing functionally active proteins on microarrays applicable to binders are known. For example, Arenkov et al. (2000) Anal. Biochem. 278: 123 teach methods of arraying functionally active proteins using microfabricated polyacrylamide gel pads. And MacBeath et al (2000) Science 289: 1760-1763 teach methods for spotting proteins onto chemically derivatized glass slides at high spatial densities. A high-precision robot was used to spot proteins onto chemically derivatized slides at high spatial densities. The proteins are attached covalently to the slide surface, yet retain their ability to interact specifically with other proteins or small molecules.

Protein or nucleic acid binder arrays of the subject invention may be created using any of the known microarray methods as reviewed in Schena et al.(ed) DNA Microarrays A Practical Approach, Oxford University Press;

Methods used for immobilizing proteins or nucleic acids applicable to the protein and nucleic acid binders of the present invention are described in the following references, and others (Mosbach (1976) Meth. Enzymol. 44:2015-2030; Hermanson, G. T. (1996) Bioconjugate Techniques, Academic Press, NY; Bickerstaff, G. (ed) (1997) Immobilization of Enzymes and Cells, Humana Press, NJ; Cass and Ligler (eds) (1998) Immobilized Biomolecules in Analysis, Oxford University Press; Watson et al. (1998) Curr. Opin. Biotech. 609:614; Ekins (1998) Clin. Chem. 44:2105-2030; Roda et al. (2000) Biotechniques 28: 492-496; Wong (1993) Chemistry of Protein Conjugation and Cross-linking CRC Boca Raton, Fla.; Taylor, (1991) Protein Immobilization: fundamentals and applications Marcel Dekker, Inc New York; Hutchens (ed) (1989) Protein recognition of immobilized ligands, Vol 83 Alan R Liss, Inc; Sleytr U. B. (ed) (1993) Immobilized macromolecules, application potentials Vol 51.Springer series in applied biology, Springer-Verlag, London; Wilchek and Bayer (eds) (1990) Avidin-Biotin Technology. Academic Press, San Diego; Ghosh et al. (1987) Nucleic Acids Res. 15: 5353-5372; Burgener et al.(2000) Bioconjug. Chem. 11: 749-754; Steel et al.(2000) Biophys J 79:975-981; Afanassiev et al.(2000) Nucleic Acids Res. 28: E66; Roda et al. (2000) Biotechniques 28: 492-496; Shena (ed) (2000) DNA Microarrays, a practical approach (Oxford University Press); Schena (ed) (2000) Microarray Biochip Technology. (Eaton Publishing Natick, Mass.); MacBeath et al. (2000) Science 289:1760-1763; Schena et al. (1998) Trends in Biotechnol. 16: 301-306; and Ramsey (1998) Nat. Biotechnol. 16: 40-44; all of which are incorporated by reference herein.

Proteins and nucleic acids have been immobilized onto solid supports in many ways. Methods used for immobilizing proteins and nucleic acids are described in the following references, and others (Mosbach (1976) Meth. Enzymol. 44:2015-2030; Weetall (1975) Immobilized Enzymes, Antigens, Antibodies and Peptides; Hermanson, G. T. (1996) Bioconjugate Techniques (Academic Press, NY); Bickerstaff, G. (ed) (1997) Immobilization of Enzymes and Cells (Humana Press, NJ); Cass and Ligler (eds) Immobilized Biomolecules in Analysis, (Oxford University Press); Watson et al. (1990) Curr. Opin. Biotech. 609:614; Ekins, R. P. (1998) Clin. Chem. 44:2105-2030; Roda et al. (2000) Biotechniques 28:492-496; Schena et al. (1998) Trends in Biotechnol. 16:301-306; Ramsay, G. (1998) Nat. Biotechnol. 16:40-44; Sabanayagam et al. (2000) Nucl. Acids Res. 28:E33; U.S. Pat. No. 5,700,637 (Southern, 1997); U.S. Pat. No. 5,736,330 (Fulton, 1998); U.S. Pat. No. 5,770,151 (Roach and Jonston, 1998); U.S. Pat. No. 5,474,796 (Brenman, 1995); U.S. Pat. No. 5,667,667 (Southern, 1997); all of which are incorporated by reference herein).

Many coupling agents are known in the art and can be used to immobilize binders in the current invention. Over 300 cross-linkers are currently available. These reagents are commercially available (e.g., from Pierce Chemical Company (Rockford, Ill.). A cross-linker is a molecule which has two reactive groups with which to covalently attach a protein, nucleic acids or other molecules. In between the reactive groups is typically a spacer group. Steric interference with the activity of the biomolecule by the surface may be ameliorated by altering the spacer composition or length. There are two groups of cross-linkers, homobifunctional and heterobifunctioal. In the case of heterobifunctional crosslinkers, the reactive groups have dissimilar functionalities of different specificities. On the other hand, homobifunctional cross linkers' reactive groups are the same. A through review of crosslinking can be found in Wong, 1993, Chemistry of Protein Conjugation and Cross-linking, CRC Press, Boca Raton. Bifunctional cross-linking reagents may be classified on the basis of the following (Pierce Chemical Co. 1994): functional groups and chemical specificity, length of cross-bridge, whether the cross-linking functional groups are similar (homobifunctional) or different (heterobifunctional), whether the functional groups react chemically or photochemically, whether the reagent is cleavable, and whether the reagent can be radiolabeled or tagged with another label.

When macromolecular ligands are used, the binders can be immobilized in such a way as to reduce steric hindrances generated by the support. A variety of methods for achieving this are known in the art. For example, the active site or other binding region of the biomolecule can be orientated away from the surface (Reviewed in Bickerstaff, (ed) (1997) Immobilization of Enzymes and Cells, pp. 261-275).

When it is necessary or desired to reduce steric problems of an immobilized binder, a suitable spacer arm or tether may optionally be used to immobilize the biomolecule to a surface. The spacer arm distances the biomolecule from the support surface. The spacer arm can be long enough to promote efficient separation of the biomolecule from the support; the spacer arm can be very flexible to provide high mobility to the immobilized biomolecule, thereby allowing maximum interaction with the macromolecule ligand. Suitable spacer arms may include, but are not limited to, dextrans, particularly those oxidized by periodate, polypeptides, protein, nucleic acids, and peptide nucleic acids, carbon spacers, polyethylene glycol polymers, and nucleic acids. For example, Maskos et al.(1992) teach methods of immobilizing oligonucleotides to chips.

Affinity biosensors are especially useful in practicing the present invention. (See, Rogers and Mulchandani (1998) Affinity Biosensors (Human Press, Totoaw, N.J.).

Other methods of protein immobilization suitable for immobilizing proteins in the subject invention involve immobilization via a fusion tail. Fusion proteins are commonly constructed having fusion tail systems to promote efficient recovery, purification, and immobilization of recombinant proteins (reviewed in Ford, et al. (1991) Protein Expr. Purif. 2: 95-107). A target protein is genetically engineered to contain a C- or N-terminal polypeptide tail, which may act as a spacer arm and provides the biochemical basis for specificity in purification and/or immobilization. Tails with a variety of characteristics have been used. Examples include entire proteins or protein domains with affinity for immobilized ligands, a biotin-binding domain for in vivo biotination promoting affinity of the fusion protein to avidin or streptavidin, peptide binding proteins with affinity to immunoglobulin G or albumin, carbohydrate-binding proteins or domains, antigenic epitopes with affinities for monoclonal antibodies, charged amino acids for use in charge-based recovery methods, poly(His) residues for recovery by immobilized metal affinity chromatography.

Recombinant DNA methodologies are commonly used to generate fusion proteins having N-terminal or C-terminal extensions that provide either a tether or spacer arm and binding sites for the immobilization of proteins. Such methods can be suitable for the immobilization of proteins and nucleic acids in the subject invention. Examples of these methods are given in the following references: Nilsson et al. (1997) Protein Expr. Purif. 11:1-16; Shpigel et al. (1999) Biotechnol. Bioeng. 65:17-23; Kroger et al. (1999) Biosens. Bioelectron. 14:155-161; Piervincenzi et al. (1998) Biosens. Bioelectron. 13:305-312; Airenne et al. (1999) Biomol. Eng. 16:87-92; Skerra, A. and Schmidt, T. G. (1999) Biomol. Eng. 16:151-156; and Jones et al. (1995) J. Chromatogr. A, 707, 3-22.

For optical biosensors solid supports such as fused silica and quartz are appropriate substrates for immobilization. Adsorption, entrapment and covalent attachment are among the techniques employed for immobilization of biomolecules onto solid supports.

Electrochemical-based enzyme immobilization methods are convenient for enzymes on microelectrodes; however, this method is restricted to use with amperometric sensors. This method allows each enzyme or nucleic acid to be located at one electrode (the working electrode). There are several situations in which conventional crosslinking based immobilization is inadequate in the construction of microelectrodes, for example, when on-wafer deposition (i.e., immobilization on the whole wafer before it is cut into smaller segments for use in individual devices) is required, leading to many localized immobilizations or during fabrication of multianalyte sensors requiring several distinct membrane sensors. The three main types of immobilization developed to overcome these problems are based on photochemistry, electrochemistry and printing (see, e.g., Bickerstaff, G. F. (ed) (1997) supra).

An immobilized binding pair member can be adsorbed, embedded or entrapped or covalently linked to surfaces. They can be adsorbed or attached to nanoparticles, for example, and these nanoparticles can be position in microflow channels. The nanoparticles can be held in position using magnetic nanoparticles and magnetic force or by a filter, grid or other support. Alternatively, the binding pair member can be adsorbed or covalently attached to the surfaces within the microflow channels or wells.

The binders can be immobilized on the surfaces within the microflow channels, wells or membranes, or the biomolecules can be immobilized onto the surfaces of beads, membranes or transducers or other surfaces placed in the flow channels, chambers or wells. Suitable beads for immobilization of binders, including proteins or nucleic acids (especially tRNAs), include chemically or physically crosslinked gels and porous or nonporous resins such as polymeric or silica based resins. Suitable media for adsorption include, without limitation, ions exchange resins, hydrophobic interaction compounds, sulfhydryls and inherently active surfaces and molecules such as plastics or activated plastics, aromatic dye compounds, antibodies, antibody fragments, aptamers, oligonucleotides, metals or peptides. Examples of some suitable commercially available, polymeric supports include, but are not limited to, polyvinyl, polyacrylic and polymethacrylate resins. Steric hindrance arising from these supports is preferably minimized. Free sulfhydryls are used in site-specific conjugation of proteins and nucleic acids to surfaces and labels.

Enzymes with quaternary structure can be used as binders in the present invention. These enzymes can undergo inactivation by dissociation of subunits and stabilization of these enzymes can be achieved by crosslinking the subunits as taught, for example, in Torchilin et al. (1983) J. Molec. Catalysis 19:291-301.

Over the past two decades, the avidin-biotin system has been developed for the immobilization of proteins, nucleic acids, as well as a wide variety of other compounds. For a review, see, Wilchek, M, and Bauer E A (ed) Avidin-Biotin Technology (Academic Press, San Diego, Calif.). Proteins or nucleic acids can be immobilized using avidin-biotin technology where a biotin labeled molecule can be bound irreversibly to avidin, which is attached to the solid support. The extraordinary affinity of avidin (or its bacterial relative streptavidin) for biotin forms the basis of this system. Since avidin, streptavidin, their analogues, and their derivatives are very stable, their immobilization is usually advantageous compared to other proteins.

Printing methods for making microarrays in the current art can be used to deliver nucleic acid or proteins to surfaces in predetermined locations. For example, aminophenyl-trimethoxysilane treated glass surfaces can bind 5′ amino-modified oligonucleotides nucleic acids using a homo bifunctional crosslinker to attach the aminated oligonucleotide to the aminated glass as taught in Guo et al. (1994) Nucleic Acids Research 22:5456-5465. Another known method for arraying nucleic acids is to react the nucleic acid with succinic anhydride and attach the resulting carboxylate group via an ethyldimethylaminopropylcarbodiimide-mediated coupling reaction (Joos et al. (1997) Anal. Biochem. 247: 96-101). In another method 5′ phosphate modified nucleic acids react with imidazole to produce a 5′-phosphoimidazolide that can bind to surface amino groups via a phosphoramidate linkage (Chu et al. (1983) Nucleic Acids Research 11:6513-6529). The linker is preferably long enough to eliminate much of the steric hindrance caused by the solid surface to ensure efficiency of the following binding reactions. For example, Shchepinov et al. (1997) Nucleic Acids Research 25:1155-1161, reported that an optimal spacer length is at least 40 atoms long can increase binding yields by 150-fold in nucleic acid hybridization experiments on microarrays.

Labels

The term “label” is used herein to refer to agents or moieties that are capable of providing a detectable signal, either directly or through interaction with additional members of a signal producing system. Labels that are directly detectable and may be used in the subject invention include, for example, fluorescent labels where fluorescers of interest include, but are not limited to fluorescein (FITC, DTAF) (excitation maxima, 492 nm/emission maxima, 516-525 nm); Texas Red (excitation maxima, 595/emission maxima, 615-620); Cy-5 (excitation maxima, 649/emission maxima, 670); RBITC (rhodamine-B isothiocyanate (excitation maxima, 545-560 nm/emission maxima, 585 nm) and others as reviewed, for example, in Haugland, R. P. (1992) Handbook of Fluorescent Probes and Research Chemicals, 5^{th} ed., Molecular Porbes, Eugene, Oreg.; radioactive isotopes, such as ³²S, ³²P, ³H, etc. Other labels can include chemiluminescent compounds, enzymes and substrates; chromogens, metals, nanoparticles, liposomes or other vesicles containing detectable substances. Colloidal metals and dye particles suitable for labels are disclosed in U.S. Pat. Nos. 4,313,734 and 4,373,932. Chemiluminescent and fluorescent labels allowing ultrasensitive assays are preferred. Labels may be detected by spectrophotometric, radiochemical, electrochemical, chemiluminescent and other means. Labels may be covalently conjugated to binding pair members.

Labels may be conjugated directly to the biorecognition molecules, or to probes that bind these molecules, using conventional methods that are well known in the arts. Multiple labeling schemes are known in the art and permit a plurality of binding assays to be performed simultaneously in the same reaction vesicle. Different labels may be radioactive, enzymatic, chemiluminescent, fluorescent, or others. Multiple distinguishable labels may be attached directly to biomolecules or they may be attached to surfaces onto which the biomolecules are immobilized. For example, beads or other particles may bear different labels, e.g., a combination of different fluorescent color dyes, that allow each bead to be independently identified. For example, Fulton et al, 1997, Clin. Chem. 43: 1749-1756, describe a standard set of 64 microspheres where each different type of microsphere is tagged with a unique combination of fluorescent dyes. Different biomolecules are immobilized to each microsphere type and reacted with their binders which are labeled with a different color fluorescent dye. The detector simultaneously identifies each bead type and the captured ligand based on the fluorescent profiles generated by the different colored fluorescent dyes.

Preferred detectable labels include enzymatic moieties capable of converting a substrate into a detectable product. Enzymes are amplifying labels (one label leads to many signals) and facilitate the development of ultrasensitive assays. For example, alkaline phosphatase and horseradish peroxidase are commonly used enzyme labels and attomole-zeptomole detection limits are routinely achieved in chemiluminescent assays with these enzymes. For alkaline phosphatase, the adamantly 1,2-dioxetane acrylphosphate substrates provide ultrasensitive assays (Bronstein et al. (1989) J. Biolumin. Chemilumin. 4:99-111). And for horseradish peroxidase, the 4-iodophenol-enhanced luminol reaction is among the most sensitive (Thorpe, et al, (1986) Methods Enzymol. 133:331-353). In such embodiments where an enzymatic label is used to convert a substrate into a detectable produce, the appropriate substrate is also added preferably after the binders have been captured on the surface.

Fluorescent labels are particularly useful in some embodiments of the current invention. By the use of optical techniques (e.g., confocal scanners, CCD cameras, flow cytometers), they permit the analysis of arrays of biorecognition elements distributed over a surface (e.g., as microdots where each microdot binds a different analyte) or differentially labeled (e.g., with beads having different combinations of fluorescent dyes).

The binding of molecules that specifically bind to a complementary binding pair member may be monitored in solution even without immobilization to a surface by attaching a fluorescent label to one or both members of the prebound binding pair and monitoring the changes in fluorescence as the binding pair members interact.

Methods for tagging or labeling proteins and nucleic acids with detectable labels are well known in the art. Radioactive and non-radioactive labels are commonly employed. For a review of enzymatic, photochemical, and chemical methods for labeling nucleic acids and proteins see Kessler (1994) J Biotechnol. 35: 165-189.

For example, reactive groups such as thiol, amine, or phosphorothioate can be introduced into nucleic acids for coupling chromophores. These methods can be applied either for the direct labeling of the binding pair members or for labeling of respective probes (DNA, RNA, oligonucleotides, aptamers, antibodies and the like). A label, e.g., a fluor, can be attached as needed to the binding pair members provided that the ability to bind ligands is not substantially diminished.

Furthermore, biotinylated binders may also be labeled in a second step using avidin or streptavidin (which bind biotin) conjugated to a fluorophor or some other label. This labeling method is commonly used in the art.

There are a number of ways to label nucleotide binders. A label may be covalently or noncovalently attached. For example, Janiak et al. (1990) Biochemistry 29: 4268-4277) labeled tRNAs by attaching fluorescein covalently to the thiouridine (s4U) at position 8 which is a conserved residue (U or s4U) in all 20 tRNAs. Synthetic and enzymatic procedures allow site specific incorporation of thionucleotide(s) within RNA (reviewed in Favre et al. (1998) J. Photochem. Photobiol. B 42:109-124). The labeled tRNAs retained their ability to be aminoacylated by the synthetases and retained their specificity and affinities for the EF-Tu:GTP binary complexes.

Alternatively, or additionally, the binding pair members may be labeled with fluor(s). For instance, modifications of various groups in proteins or peptides with fluors are summarized in a variety of reviews and monographs (for example, see Haugland, R. P. (1992) Handbook of Fluorescent Probes and Research Chemicals, 5^{th} ed., Molecular Probes, (Eugene, Oreg.)). Although several groups can be used to couple a label, the thiol group is thought to be the best candidate in that many functional groups used to attach labels are thiol-specific or selective, and thus unique labeling is possible. For example, with site directed mutagenesis, a thiol group can be added to or deleted from a desired position (Cornish et al. (1994) Proc. Natl. Acad. Sci. USA 91: 2910-2914). Other groups on proteins surfaces commonly used for the conjugation of a label are amines (e.g., from surface lysines).

Radiolabeled and fluorescently labeled nucleotide triphosphates are commonly used in biology and are commercially available from a number of sources.

Multianalyte Testing

Simultaneous multianalyte testing is now possible, and any known method for multianalyte analysis can be used to construct analyzers employing a plurality of diverse sets of binding pair members providing the biomolecular recognition elements. Methods of simultaneous multianalyte testing include assays based on more than one label and assays based on spatially separated reaction zones. For example, researchers have used binders in the same assay zone labeled with different fluorescent molecules (Vuori et al. (1991) Clin. Chem. 7:2087-2092; Hemmila, I. (1987) Clin. Chem. 33:2281-2283), different radioactive species (Wians et al. (1986) Clin. Chem. 32:887-890; Gutcho et al. (1977) Clin. Chem. 23:1609-1614; Gow et al. (1986) Clin. Chem. 32:2191-2194), different enzymes (Nanjee et al. (1996) Clin. Chem 42:915-926), metal ions (Hayes et al. (1994) Anal. Chem. 66:1860-1865), colored latex particles (Hadfield et al. (1987) J. Immunol. Methods 97:153-158) and particles of different sizes (Frengen et al. (1995) J. Immunol. Methods 178:141-151). Various detection schemes employed in these multianalyte may be based on changes in one or more of the following signals: absorbance, steady-state fluorescence, fluorescence lifetime, chemiluminescence, radioactivity, electrochemical response, laser light scattering, and frequency of a piezoelectric quartz crystal, upon the binding event(s).

Microfluidics

Microfluidic/microflow-or microscale refers to the handling and/or provision of fluids of an amount consistent with the capillary dimensions as outlined here. Capillary dimension is the capillary cross-sectional area that provides for capillary flow through a channel. At least one of the cross-sectional dimensions, e.g., width, height, diameter, is at least about 1 micron usually at least 10 microns and is usually no more than 500 to 1000 microns. Channels of capillary dimension have an inside bore diameter (ID) of less than about 1 millimeter and are typically from about 1 to 200 microns, more typically from about 25 to 100 microns.

Microfluidic/microflow or microsystem or microscale processing refers to processing of fluids on a microfluidic scale. The processing involves fluid handling, transport and manipulation within chambers and channels of capillary dimension. Valveless sample injection is achieved by moving fluid from the reagent reservoirs into cross-channel injection zones, where plugs of buffer or test compounds are precisely metered and dispensed into a desired flowpath. The rate and timing of movement of the fluids in the various microchannels can be controlled by electrokinetic, magnetic, pneumatic, and/or thermal-gradient driven transport, among others. These sample manipulation methods enable the profile and volume of the fluid plug to be controlled over a range of sizes with high reproducibility. In addition, microfluidic processing may include sample preparation and isolation where enrichment microchannels containing separation media are employed for target capture and purification. Microfluidic processing may also include reagent mixing, reaction/incubation, separations and sample detection and analyses.

For the purpose of this invention the terms “microchannel”, “channel”, “capillary”, “miniaturized flow channel” and “microflow channel” may be understood to be interchangeable.

“Channel” refers to a conduit or means of communication, usually fluid communication, more particularly, liquid communication, between elements of the present apparatus. The channel may be an enclosed space or cavity of dimensions generally between 1 mm and 1 micron. The channels can also be capillaries.

In general the channel has an inlet port, an outlet, port and may have binders covalently or non-covalently attached to the surface. Channels include capillaries, grooves, trenches, microflumes, and so forth. The channels may be straight, curved, serpentine, labyrinth-like or other convenient configuration within the planar substrate. The cross-sectional shape of the channel may be circular, ellipsoid, square, rectangular, triangular and the like so that it forms a microchannel within the planar substrate in which it is present.

The term “in fluid communication” defines components that are operably interconnected to allow fluid flow between components.

The inside of the channel may be coated with a material for strength, for enhancing or reducing flow, for enhancing detection limits and sensitivity, and so forth. Exemplary of coatings is silylation, polyacrylamide (vinyl bound), methylcellulose, polyether, polyvinylpyrrolidone, and polyethylene glycol, polypropylene, Teflon.TM. (DuPont), Nafion.TM. (DuPont), and the like may also be used.

Fluid material may be transported from the reservoirs to the reaction channels and throughout the microflow systems by various methods known in the arts. Miniaturized mechanical pumps, based on microelectromechanical systems (MEMS) can be employed. Examples of micro fluidic transport devices that may be used in the subject invention include pneumatically or hydraulically driven systems or microfabricated pumps and/or valves, for example, as reviewed in (ShoJi, S et al (1994)“Microflow devices and systems” J. Micromech. Microeng. 4:157-171).

Alternatively centrifugal or electrokinetic transport mechanisms may be employed., for example as described in U.S. Pat. No. 5,585,069 issued Dec. 17, 1996 to Zanzucchi et al and in U.S. Pat. No. 4,908,112 issued to Pace Mar. 13, 1990 and taught in Dasgupta et al (1994) “Electroosmosis: A reliable fluid propulsion system for flow injection analysis”, Anal. Chem. 66, p 1792-1798 or electrophoresis methods, which require inert metal electrodes. Magnetic forces may be used to move a sample or to immobilize a paramagnetic bead-binder complex.

The devices of the current invention can be made by a variety of processes including but not limited to lasering, embossing, photolithography, casting, electroplating, and micromachining. The methods utilized to manufacture the structures of the current invention are not critical.

A reaction microflow channel may have a variety of configurations and may be sufficiently long to allow reaction of the sample with the immobilized binder to facilitate an affinity elution event. The reaction channel may be integrated with a detector that may continuously monitor the amount of affinity eluted or biospecifically desorbed material. The reaction channel may optionally comprise, and usually may comprise fluid reservoir arrays as described above.

A waste fluid reservoir may optionally be present for receiving and storing the waste portion of the sample volume that flows from the outlet.

The subject microsystems may optionally comprise an interface means for the delivery of a sample. For example, the Microsystems may have a syringe interface which serves as a guide for a syringe needle and as a seal.

In one embodiment, the subject systems are integrated Microsystems. By integrated is meant that all of the components of the system (with the exception of the detector and computer) are present in a compact unit such as a chip miniaturized flow system, disk or the like and that many functions traditionally performed by a technician, including the addition of reagents by pipetting, incubation, and data acquisition and processing, may be performed automatically under computer control.

Integrated microflow systems for studying biospecific interactions and their inhibitors of the subject invention require a highly controlled means of manipulating fluids and substances within them and transporting the fluids through microreactors where chemical and biochemical reactions or partitioning take place. The reaction microchannels, those channels bearing the immobilized binding complex of interest, are in fluid connection with various reservoirs and with a detector(s) to monitor the biospecific interactions.

The devices of the subject invention may be fabricated from a variety of materials, including fused silica, glass, acrylics, thermoplastics, and other polymers including polymethylmethacrylate, polycarbonate, polyethylene terepthalate, polystyrene, styrene copolymers, and others. The different components and devices of the integrated microsystems may be fabricated from different materials. The microflow channels may be present on the surface of a planar substrate and the substrate may be covered by a planar cover plate to seal the microchannels present on the surface. The devices may be small with the longest dimensions being about 250 mm. The devices may have any convenient configuration including capillary, disk, chip, or syringe-like and others.

The systems and devices of the current invention may be fabricated using any convenient means known in the arts, including, but not limited to molding and casting for example as disclosed in U.S. Pat. No. 5,110,514. The use of polymeric materials in the fabrication of microfluidic devices is also described in U.S. Pat. No. 5,885,470

Microfluidic Arrays

Microarrays (i.e., arrays on a microscale) of microfluidic/microflow systems are a further aspect of the invention. These systems may analyze tiny amounts of samples with high sensitivity. These systems advantageously can offer femtomole or attomole concentration detection, which sensitivity is made possible by the use of fluorescence detectors that possess higher sensitivities than typically present in such analyzers.

Arrays permit many assays to be performed in parallel. For example, array-based biosensors are used for multianalyte sensing (see Michael K. L et al, (1998) Anal Chem 70: 1242-6).

Current methods for multianalyte analysis can be classified into two formats, assays, based on more than one label, and assays, based on spatially separated zones, for each biorecognition molecule specific for a different analyte. Biorecognition elements that recognize different analytes may be immobilized on spatially separated zones or positioned into separate chambers and the assays may be monitored simultaneously using position-sensitive detectors (for review, see Ekins, R P (1998) Clin. Chem 44:2015-30).

Microarrays useful in the present invention vary according to their transduction mechanisms and include surface acoustic wave sensors, microelectrodes, solid-state sensors, and fiber-optic sensors. However, optical, electrochemical and piezoelectric crystal arrays are preferred. These systems may be used to analyze amino acids in volumes of less than 1 microliter with a sensitivity many orders of magnitude greater than current amino acid analyzers.

It is now possible to fabricate complex miniaturized systems. This technology represents a combination of several disciplines that include microfabrication, microfluidics, microelectronmechanical systems, chemistry, biology, and engineering. Miniaturized devices can be electrical, such as microelectrodes and signal transducers; optical such as photodiodes and optical waveguides; and mechanical, such as pumps. In the new field of microfluidics, the integration of automated microflow devices and sensors allow very precise control of ultra-small flows on microchip platforms (Gravesen et al. (1993) J. Micromech. Microeng. 3:168-182; Shoji and Esashi (1994) J. Micromech. Microeng. 4:157-171). Many different flows can be combined in all sorts of ways and mixed on the same chip. Existing technology also allows the integration of intersecting channels, reaction chambers, mixers, filters, heaters, and detectors to perform on-chip reactions in sub-nanoliter volumes in a highly controlled and automated manner with integrated data collection and analysis (Colyer et al. (1997) Electrophoresis 18:1733-1741; Effenhauser et al. (1997) Electrophoresis 12:2203-2213).

A variety of different microarrays and detectors can be employed in the practice of the present invention. Arrays used in the subject invention can be biosensor, microparticle, microbead, microsphere, microspot, microwell, microfluidic arrays, and the like. The substrates for the various arrays can be fabricated from a variety of materials, including plastics, polymers, ceramics, metals, membranes, gels, glasses, silicon and silicon nitride, and the like. The arrays can be produced according to any convenient methodology known to the art. A variety of array and detector configurations and methods for their production are known to those skilled in the art and disclosed in U.S. Pat. Nos. 6,043,481; 6,043,080; 6,039,925; 6,025,129; 6,025,601; 6,023,540; 6,020,110; 6,017,496; 6,004,755; 5,976,813; 5,872,623; 5,846,708; 5,837,196; 5,807,522; 5,736,330; 5,770,151; 5,711,915; 5,708,957; 5,700,637; 5,690,894; 5,667,667; 5,633,972; 5,653,939; 5,658,734; 5,624,711, 5,599,695; 5,593,839; 5,906,723; 5,585,639; 5,584,982; 5,571,639; 5,561,071; 5,554,501; 5,534,703; 5,529,756; 5,527,681; 4,472,672; 5,436,327; 5,429,807; 5,424,186; 5,412,087; 5,405,783; 5,384,261; 5,474,796; 5,274,240; and 5,242,974. The disclosures of these patents are incorporated by reference herein.

The arrays may be positioned into the bottom of microwells, microchannels or on the surfaces such as planar waveguides. The area of Micro-Total Analysis Systems (mu TAS), otherwise known as “Microsystems” or “Lab-on-a-chip”, is used to describe miniaturized sensing devices and systems that integrate microscopic versions of the devices necessary to process chemical or biochemical samples, thereby achieving completely automated and computer controlled analysis on a microscale. Micro/miniaturized total analysis systems developed so far may be classified into two groups. One is a MEMS (Micro Electro Mechanical System), which uses pressurized flow controlled by mechanical flow control devices (e.g., microvalves, micropumps or centrifugal pumps). The other types use electrically driven liquid handling without mechanical elements. Currently, microsystems are being produced in both academic and commercial settings. The tern “microsystem” is used herein to describe both types of miniaturized systems. A variety of integrated Microsystems, MEMS, and microsystem devices are well known to the art. See, for example, U.S. Pat. Nos. 6,043,080; 6,042,710; 6,042,709; 6,036,927; 6,037,955; 6,033,544; 6,033,546; 6,016,686; 6,012,902; 6,011,252; 6,010,608; 6,010,607; 6,008,893; 6,007,775; 6,007,690; 6,004,515; 6,001,231; 6,001,229; 5,992,820; 5,989,835; 5,989,402; 5,976,336; 5,972,710; 5,972,187; 5,971,355; 5,968,745; 5,965,237; 5,965,001; 5,964,997; 5,964,995; 5,962,081; 5,958,344; 5,958,202; 5,948,684; 5,942,443; 5,939,291; 5,933,233; 5,921,687; 5,900,130; 5,887,009; 5,876,187; 5,876,675; 5,863,502; 5,858,804; 5,846,708; 5,846,396; 5,843,767; 5,750,015; 5,770,370; 5,744,366; 5,716,852; 5,705,018, 5,653,939; 5,644,395; 5,605,662; 5,603,351; 5,585,069; 5,571,680; 5,410,030; 5,376,252; 5,338,427; 5,325,170; 5,296,114; 5,274,240; 5,250,263; 5,180,480; 5,141,621; 5,132,012; 5,126,022; 5,122,248; 5,112,460; 5,110,431; 5,096,554; 5,092,973; 5,073,239; 4,909,919; 4,908,112; 4,680,201; 4,675,300; and 4,390,403, all of which are incorporated by reference herein.

Techniques for detection of analytes in the integrated microsystems and microarrays include, but are not limited, to fluorescence emissions, optical absorbance, chemiluminescence, Raman spectroscopy, refractive index changes, acoustic wave propagation measurements, electrochemical measurement, and scintillation proximity assays. There are many demonstrations in the literature of single molecules being detected in solution using fluorescence detection. A laser is commonly used as an excitation source for ultrasensitive measurements and the fluorescence emission can be detected by a photomultiplier tube, photodiode or other light sensor. Array detectors such as charge coupled device (CCD) detectors can be used to image the analytes spatially distributed on an array. Laser-induced fluorescence is generally the detection method of choice for microarray and microflow systems. There are many examples in the literature describing single molecule detection using laser-induced fluorescence as a detection method. For example, spatially resolved detection may be achieved using confocal laser scanners or high sensitivity imaging detectors such as CCD cameras.

Several microchip fluorescent detection systems are commercially available. These include the Hewlett Packard's BioChip Imager with epi-fluorescence confocal scanning laser system having a 50 micrometer, 20 micrometer, or 10 micrometer resolution. This instrument detects less than 11 molecules of the dye Cy5/square micrometer and has a dynamic range of four orders of magnitude. General Scanning's ScanArray 3000 is a scanning confocal laser with a 10 micrometer resolution that can detect 0.5 molecule of fluorescin/micrometer² (or less than 0.15 attomole of end labeled nucleotide) taking 4 minutes to scan a 10 micrometer by 10 micrometer chip. Molecular Dynamics' Avalanche confocal scanners have a resolution of 10 micrometers and can detect less than 10 molecules of Cy3 molecules/square micrometer on chips taking 5 minutes to scan the entire chip.

Methods for the spatially resolved and ultrasensitive detection of fluorescently labeled molecules in microfluidic channels are disclosed, for example, in U.S. Pat. Nos. 5,933,233 and 6,002,471. Instrumentation for the detection of single fluorescent molecules is described in U.S. Pat. No. 4,979,824 and reviewed in Sinney et al, (2000) J Mol Recognit, 13, 93-100; Nie, S. and Zare, R. N. (1997) Ann. Rev. Biophys. Biomol. Struc. 26, 567-96; Rigler, R. (1995) J Biotechnol. 41, 177-186; Chan, W. C. and Nie, S (1998) Science 281, 2016-8; and Nie, S. and Emory, S. R. (1997) Science 275, 1102-6. CCD imagers for confocal scanning microscopes are disclosed in U.S. Pat. Nos. 5,900,949, 6,084,991, and 5,900,949. Capillary array confocal scanners are described in U.S. Pat. No. 5,274,240. CCD array detectors suitable for microchips are described in U.S. Pat. Nos. 5,846,706, and 5,653,939. Detector systems for optical waveguide microarrays are disclosed in U.S. Pat. Nos. 6,023,540, 5,919,712, 5,552,272, 5,991,048, 5,976,466, 5,815,278, 5,512,492.

Mass sensing biosensors such as piezoelectric sensors are known, for example, as disclosed in U.S. Pat. Nos. 4,236, 4,735, and 6,087,187 and are suitable for use in the present invention to construct amino acid biosensor arrays.

Microtiter Arrays

Rapid, automated and simultaneous testing of multiple samples are commonly performed in microwell formats. The microtiter plate has become a popular format for biological assays because it is easy to use, is readily integrated into an automated process and provides multiple simultaneous testing on a simple disposable device. The traditional 96-well format is being replaced with microwells with larger numbers of smaller wells. These provide plates with 192-20,000 wells with volumes that range from 125 microliters to 50 nanoliters (Reviewed in Kricka (1998) Clinical Chemistry 44:2008-2014). A range of new micropipetting systems based on inkjet principles have been developed for delivery of nanoliter volumes of samples and reagents to microwells (for example, see, Rose and Lemmo (1997) Lab Automat News: 2:12-9; Fischer-Fruholz (1998) American Lab; February 46-51). The new high-density, low volume microwell format has been adapted for a diverse range of analytical methods. Most are simple homogeneous assays such as scintillation proximity assays, fluorescence polarization assays, time resolved fluorescence, fluorescence energy transfer, and enzyme assays.

Advantageous properties of substrates for the microarrays of the subject invention are those for substrates of traditional microarrays: ease of manufacture and processing, compatibility with detection systems, good material strength, and low nonspecific biomolecule adsorption. The substrate material preferably allows efficient immobilization of biomolecules either directly or through an intermediate surface coating. Glass, silicon, and plastic substrates are commonly used for microarray production and are examples of suitable substrates for use in some preferred embodiments of the subject invention. Glass has a number of favorable qualities. These include transparency, and the compatibility with radioactive and fluorescent samples. However, a variety of other materials are suitable substrates. Polypropylene also has favorable physical and chemical properties. For example, Boehringer Mannheim uses small disposable polystyrene carriers onto which microdots are deposited using inkjet technology (Ekins (1998) Clin. Chem. 44:2015-2030). As mentioned above, biomolecule immobilization on chips may be accomplished by various means including, but not limited to, adsorption, entrapment, and covalent attachment. Covalent attachment is the preferred method for “permanent” immobilization. Functionalized organosilanes have been used extensively as an intermediate layer for biomolecule immobilization on glass and silicon substrates. Silanes are commercially available that contain an ever-increasing number of reactive functional groups suitable for biomolecule conjugation either directly or via a cross-linker.

For interaction analysis, a flow system is superior to static microwell formats. Microflow devices permit the control of fluids in channels of micron dimensions (typically 10-1000 micrometers in diameter). These lab-on-a-chip systems measure and distribute fluids; chemicals mix and react is they flow through the channels; temperature and reaction times are controlled; and the results are automatically detected, analyzed and displayed. Flow through sensors offer may advantages over probe type sensors. Flow systems facilitate sample transport and conditioning, as well as calibration. Microflow systems are especially well suited for studying biospecific interactions. Microflow systems permit binding assays without washing or incubation steps, yield highly reproducible results, are easy to calibrate and automate, and allow automated and precise addition of reagents with automated data acquisition, analysis and computer controlled feedback fluidic manipulations.

Microarray Printing Technologies

The microarrays of the current invention can be made using existing technologies for array construction. The microarrays of the current invention may be produced, for example, by deposition of tiny amounts of a binder or binder member pair solution in a predetermined pattern on a surface using arraying robots (As reviewed, for example, in Schena (ed) (2000) “Microarray Biochip Technology” Eaton Publishing, Natick, Mass.; Schena (ed) (2000) “DNA Microarrays A Practical Approach”, Oxford University Press). The volume delivered is typically in the nanoliter or picoliter range.

The technologies for spotting arrayed materials onto a substrate fall into two categories: noncontact and contact dispensing. Noncontact dispensing involves the ejection of drops from a dispenser onto the surface. Contact printing involves direct contact between the printing mechanism and the solid support. For example, to construct binder member or prebound binder pair microarrays of the current invention, a high-precision contact-printing robot may be employed to deliver nanoliter volumes of the binders or prebound binder pairs to surfaces yielding spots preferably of about 150 to 200 micrometers in diameter.

A variety of chemically derivatized substrates can be printed and imaged by commercially available arrayers and scanners. For example, slides that have been treated with an aldehyde-containing silane reagent are commonly available (e.g., from TeleChem International, Cupertino, Calif.). The aldehydes react with primary amines on proteins or amine modified nucleic acids to form a Schiff's base linkage. Substrates for microarray construction may be coated by a protein layer and the proteins to be spotted may be attached to this protein layer using chemical crosslinking. For example, MacBeath et al. (2000), supra, teach a method for spotting proteins on microarrays. The proteins are printed in phosphate-buffered saline with 40% glycerol included to prevent evaporation of the nanodroplets. They attached a layer of bovine serum albumin (BSA) to the surface of a glass substrate. Glass treated with an aldehyde-containing silane reagent readily react with amines on a protein's surface to form a covalent attachment forming a molecular layer of BSA. The BSA on the surface is then activated using a chemical cross-linking reagent (e.g., N,N′-disuccinimidyl carbonate). The activated residues on the BSA then react with residues on the printed protein to form covalent linkages. Printed proteins are displayed on top of the BSA monolayer rendering them accessible to macromolecules in solution.

Another example of a known method for microarray construction involves the in situ synthesis of unique oligonucleotides on a solid support. Proteins or other biomolecules may be attached to oligonucleotides having complimentary sequences to those positioned on the array in known locations. These oligonucleotide bearing biomolecules are then bound to the arrays in known locations by complimentary base pairing (for a review of this method, see, Niemeyer et al.(1998) Analytical Biochem. 268, 54-63)

Microflow Systems

The microsystem can be divided into two parts: the mechanical portion with the biochemistry and microfluidic pumps and the electronic portion which has the laser, detector, and the computer interface.

In one preferred embodiment, the computer interface can be approached by building a custom circuit which connects to a plurality of light detectors and other input timing signals. The custom circuit would be a stand alone microprocessor which collects all of the timing and light intensity information and sends the resulting data out to a computer, for example, via a USB or serial port. The computer can be programmed for data analysis.

Because diffusion in liquids is random and slow over distances greater than a few micrometers, the incorporation of arrays into flow systems for automated processing facilitates high throughput analysis and permit sequential monitoring. Solid-phase ligand assays are currently performed in microtiter plates; however, this technique requires long incubation times to achieve equilibrium conditions and is difficult to miniaturize and automate. By contrast, flow systems are easily automated and miniaturized and allow fine control of reagent additions and rapid chemistries by reducing diffusion limitations. In addition, reproducibility is extremely high and calibrations are easy to perform (Scheller et al. (1997) Frontiers in Biosensors. 1. Fundamental Aspects, Birkhauser Verlan, Basel, Switzerland). When coupled with microdialysis and flow injection systems, biosensors have become available for on-line, real-time monitoring (Freaney et al. (1997) Ann. Clin. Biochem. 34:291-302; Cook, J. (1997) Nat. Biotech. 15:467-471; Steele and Lunte (1995) J. Pharm. Biomed. Anal. 13:149-154; Kaptein et al. (1997) Biosens. Bioelectron. 12:967-976; Nima et al. (1996) Anal Chem. 68:1865-1870).

The delivery of microliter to nanoliter volumes of samples to the arrays of the present invention can be achieved using recently developed micropipetting systems (Rose and Lammo (1997) Automat. News 2:12-19).

Note the microflow system may be constructed using multiple capillaries as well as multiple microchannels. In the present context, the word channel means channel or capillary. The microchannels or capillaries of the present invention can be from 1-1000 microns in diameter.

In some preferred aspects of the invention, the fluidic system allows automated calibration with known concentrations of analytes, prewashing with equilibration buffer, incubation with any necessary factors, and postwashing to remove unbound material and regenerate the sensor chip all under computer control. Fluidic handling (volumes and flow rates of the respective solutions) and data acquisition or image acquisition (series of fluorescence images) can be synchronized by means of a computer.

Detectors

A variety of methods and means can be used to detect and/or quantify the affinity eluted substance in the subject invention. Techniques envisaged for such detection or measurement include fluorescence emission, chemiluminescence, optical absorbance, refractive index changes, various forms of Raman spectroscopy, electrochemical amperiometric measurement, acoustic wave propagation measurements, and conductometric measurements. Laser induced fluorescence is an extremely sensitive detection method and single molecules have been detected in microchannels using this technique. A laser is often used as an excitation source for ultrasensitive measurements. The fluorescence emission may be detected by a photodiode, a photomultiplier tube or other light detector. An array detector such as a confocal scanner or a charge-coupled device (CCD) detector can be used providing spatially specific detection.

The micro fluidic systems may include an optical detection window disposed in the structure of the system adjacent to one or more of the microchannels. Optical elements may be either fabricated into the body structure or attached to the body structure such that the optical elements form a single integrated unit with the body structure. Examples of optical elements that may be used in the current invention include optical fibers, lenses, optical filters, optical gratings, beam splitters, mirrors, polarizers, waveguides and the like. The use of these optical elements are taught in Handbook of Optics, volume 11,1995, McGraw-Hill, for example. The optical elements may be fabricated into a substrate layer making up the body structure of the device. Alternatively a scanning detector (e.g. a confocal scanner) or an imaging detector (e.g a CCD camera) may be used.

Appropriate light sources include, for example, lasers, LEDs, laser diodes, high intensity lamps and the like. The light energy may be transported from the source to the channel and the emission light transported back to the detector via optical fibers or other optical waveguides. Optical detection cells for microfluidic devices are described, for example, in U.S. Pat. No. 5,599,503 issued to Manz et al Feb. 4, 1997.

Detectors useful in the present invention vary according to their transduction mechanisms and include surface acoustic wave sensors, microelectrodes, solid-state sensors, and fiber-optic sensors. However, optical, electrochemical and piezoelectric crystal detectors are preferred. These systems may be used to analyze samples in volumes of less than 1 microliter with a sensitivity many orders of magnitude greater than current instrumentation.

A biosensor can also be used as a detector. The biosensor can be a self-contained integrated device that is capable of providing quantitative or semi-quantitative analytical information using a biological recognition element which is in direct contact with a transduction element. For a review of real time, miniaturized sensors; see, e.g., Rogers and Mulchandani (1998) Affinity Biosensors: Techniques and Protocols, Humana Press, Totawa, N.J. Biosensors can be classified according to their transduction mechanisms and include microelectrodes, surface acoustic wave sensors, and fiber optic sensors. A commercially available biosensor system called BIAcore (Pharmacia Biosensor, Uppsala, Sweden) contains a sensor microchip, a laser light source emitting polarized light, an automated fluid handling system, and a diode-array position sensitive detector (Raghavan and Bjorkman (1995) Structure 3:331-333). This system uses a surface plasmon resonance assay, an optical technique that measures changes in the refractive index at the sensor chip surface. These systems can monitor biological interaction phenomena at surfaces in real-time under continuous flow conditions.

Any of the usual energy transduction modes can be fabricated in an array format and used to construct amino acid analysis biosensor arrays. Each biorecognition element can be placed on transducers which monitor mass changes, the formation of electrochemical products, or the presence of fluorescence. Optical and electrochemical transducers, however, provide the most sensitive biosensors and are well suited for miniaturization and are thus advantageous in the practice of the present invention.

In particular, detection systems for capillary arrays and microchannel arrays are known in the art (Huang et al. (1992) Anal. Chem. 64:967-72; Mathies et al. (1992) Anal. Chem. 64:2149-54; Kambara et al. (1993) Nature 361:565-566; Takahashi et al. (1994) Anal. Chem. 66:1021-1026; Dovichi et al. (1994) In: DOE Human Genome Workshop IV, Santa Fe, N. Mex., November 13-17 Abstract #131; Wooley et al. (1994) Proc. Natl. Acad. Sci. USA 91:11348-52; Wooley et al. (1997) Anal. Chem. 69:2181-21866; Simpson et al. (1998) Proc. Natl. Acad. Sci. USA 95:2256-2261; Schmalzing et al. (1998) Anal. Chem.70:2303-10; Ueno, K. (1994) 66:1424-31; Lu et al. (1995) Appl. Spectrosc. 49:825-833).

In certain preferred embodiments, the microfluidic system can use side-entry laser irradiation and irradiate all the microflow channels simultaneously. Detection can be achieved with a highly sensitive camera system from a direction perpendicular to the incident laser beam. The fluorescence from the irradiated region produces a line image on the CCD detector, which may be a cooled CCD camera coupled with a cooled image intensifier and this detector is connected to a computer. The excitation light source may be a He—Ne laser. The excitation wavelength can depend on the assay type and fluorophore(s) used. The laser beam can be focused at the outlet of the parallel channels to excite the fluor(s) as they flow out of the channel array. A light emitting diode can also be used as a light source for exciting a fluorescent detectable tag. A photomultiplier tube can be used in the detection system or the excitation light source.

Any of the transducers used in biosensors can be engineered in an array format and used to monitoring the displacement of the prebound binding pair member. Recent developments in engineering have improved transducer piezoelectric technology, leading to a new generation of sensor devices based on planar microfabrication techniques. Piezoelectric biosensors (see, e.g., Ghidilis et al. (1998) Biosens. Bioelectron. 13:113-31; Suleiman et al. (1994) Analyst 119:2279-82; Karube et al. (1988) U.S. Pat. No. 4,786,804) are well suited to miniaturization and detect femtomole levels of analyte. In addition, labeling of the analyte is not necessary. Surface plasmon resonance biosensors are commercially available and can monitor biomolecular interactions in real time during continuous flow.

Piezoelectric biosensors and surface plasmon-based biosensors for amino acids are within the scope of detectors useful in the practice of the present invention. Piezoelectric crystals and surface plasmon resonance biosensor formats are envisaged for amino acid analysis in the subject invention. The biorecognition elements can be immobilized onto piezoelectric crystals for example, according to the methods of Storri et al. (1998) Biosens. Bioelectron. 13:347-57 and Lu H. C. et al. (2000) Biotechnol. Prog. 13: 347-57. Piezoelectric array biosensors have been described. (Wu, T. Z. (1999) Biosens. Bioelectron. 14:9-180).

In general, any object that acts as a waveguide can be engineered into an evanescent wave biosensor. Planar waveguide biosensor arrays have been described (Rowe-Taitt et al. (2000) Anal. Biochem. 231:123-133; Rowe et al. (1999) Anal. Chem. 71:3846-52; Rowe et al. (1999) Anal Chem. 71:433-9; Flora et al. (1999) Analyst 124:1455-62; Herron et al.(1999) U.S. Pat. No. 5,919,712).

Scintillation proximity assays are envisaged. In scintillation proximity assays, a radioisotope is used as an energy donor and a scintillant-coated surface (e.g., a bead) is used as an energy acceptor. Scintillation proximity assays (SPA) are described in U.S. Pat. No. 4,568,649 which is incorporated herein by reference. The binding pair member can be bound to SPA beads (commercially available from Amersham Corp., Amersham Place, Little Chalfont, England). For example, a biotinylated binding pair member may be conjugated to avidin or streptavidin coated SPA beads. Biotin in the form of N-hydroxysuccinimide-biotin is available from Pierce Chemical Co., Rockford, Ill. This embodiment comprises an acceptor SPA beads and quantitation of the radiolabeled prebound binding pair member on a scintillation counter (for example, a microchip or microplate scintillation counter).

Microtiter plate formats using fluorescent labels and microplate fluorometers enable femtomole-attomole sensitivities. Many types of microplate fluorometers are commercially available. Molecular Device's FLIPR or LJJ Biosystem's Acquest have the ability to handle 1536-well plates and have a high degree of automation. Bio-Tek Instruments model FL600 microplate fluorometer can detect less than 2 femtomoles of fluorescein with a read time of 28 sec. Molecular Device's SPECTRAmax Gemini microplate fluorometer can detect 5.0 femtomoles of FITC in 96 well plates with a read time of less than 27 sec. Instruments are also available that combine time-resolved fluorescence with fluorescence resonance energy transfer pairing. This combination requires two fluorophores emitting at different wavelengths. The first emits right away, but the second is activated only when the two are in proximity, i.e., when two labeled molecules are bound. This allows simultaneous measurement of bound and unbound analytes and thus permits internal calibration. As mentioned above, it also means that the assay is homogeneous, and therefore, it is easy to automate and miniaturize.

Other detectors suitable for use in the current can depend on the label employed. The labels can be quantitatively detected in a manner appropriate to their nature, for example, by counting the radioactivity of a radioactive label or scanning a fluorescent label with a light beam. Detectors include, but are not limited to, scintillation counters, e.g., a microplate scintillation counter such as TopCount (Packard), gamma counters, phosphorimagers, luminometers, spectrofluorometers, spectrophotometers and others.

In addition to data acquisition with commercial microplate spectrophotometers, energy transfer assays of the subject invention can be incorporated into automated microfluidic assays for ultrasensitive and high throughput amino acid analysis (see, for example, Mere et al. (1999) Drug Discov. Today 4:363-369). FRET assays are also performed using commercial flow cytometers as described in Song et al.(2000) Anal. Biochem. 284:35-41; Burando et al.(1999) Cytometry 37: 21-31

Optical detection methods, especially those employing fluorescence detection, are preferred in some embodiments of the current invention. In general, a fluor bound to elements of the microarray is visualized by fluorescence detection. Confocal scanners and CCD cameras are commonly employed for detection in microarrays and may be used in the subject invention.

Confocal scanners use laser excitation of a small region of the viewing area and the entire image is obtained by moving the substrate or the confocal lens (or both) across the viewing area in two dimensions. Light emitted from the fluorescent sample at each position in the microarray is separated from unwanted light by employing a series of mirrors, filters, and lenses. The light is then converted into an electronic signal with a light detector (e.g., a photomultiplier tube (PMT)).

Fluorescence imaging with a CCD camera is also employed for detection in microarrays. CCD-based imaging often employs illumination and detection of a large portion of the viewing area (e.g., 1 cm²) simultaneously. Filtering methods of emission spectra in CCD based systems minimize optical cross-talk between different channels. Detailed descriptions of confocal scanners and CCD imaging systems are provided in Schena (ed) (2000) DNA Microarrays—A Practical Approach, (Oxford University Press).

The fluorescent emission from the microarray is converted into a digital output by the detection system. The data are quantitated and interpreted. Quantitation may be accomplished by superimposing a grid over the microarray image and computing the average intensity value for each microarray element using automated software. The intensity values are then converted into amino acid concentrations by comparing the experimental and control elements.

Excitation light can be generated by a variety of sources such as lasers, arc or filament lamps, or LEDs. The excitation light is directed into the microarray sample. This can be accomplished in a number of ways. For example, a flood illumination manner, where a large area of the sample is excited at one time, may be used. Flood illumination is most often used with CCD camera type instruments. Alternatively, the excitation light may be focused to a small spot to illuminate a small portion of the sample. In some embodiments, excitation light may be transported to the microelements, which may be microchannels, using optical fibers or other waveguides.

Excitation wavelengths are chosen based on the dyes employed. For example, fluorescein isothiocyanate (FITC) is one example of a dye that may be used in the subject invention. The excitation maximum is about 493 nm and the emission maximum is about 516-525 nm. The excitation wavelength cannot be too close to the emission peak or it can pollute the fluorescence signal. For FITC, that suggests excitation wavelengths between 470-495, for example. Fluorescence measurements will use appropriate excitation/emission filter sets for each dye employed.

Biomolecules can exhibit conformation changes upon the binding of analyte which can easily be detected by a fluorescence change. Concerns about the stability of biosensors incorporating proteins can be addressed by using thermostable proteins which provide a longer life time. The development of new technologies such as polarization-based sensing and life-time based sensing which, for example, can be accomplished with light emitting diodes as a light source can provide a biosensor that are specific.

Light Collection

The fluorescent light is most often gathered or collected by an objective lens. This lens focuses on the sample and directs emitted light within some angular range into a detection path. Spatial addressing may be achieved by using a multielement detector array, such as a CCD camera, placing light detectors in microflow channels, delivering light to microflow channels using a unique optical fiber for each channel, emission light may travel back through the same optical fiber to the detector. CCD cameras may be configured to stare at an area that has been flood illuminated. Alternatively, mechanical scanning may be employed. This can be done by scanning the light beam with mirrors, moving the sample or a combination of both.

Collectors include photomultiplier tubes, CCD cameras, and avalanche photodiodes, for example. Light collectors or detectors are also employed when using chemiluminescent labels, but an excitation source is not needed in this case.

Excitation/Emission Discrimination

In order to detect the fluorescence signal from the emission light some optical means is incorporated to separate the two types of light. Emission filters are typically placed in the emission beam before the detector. These are interference filters that pass a narrow band of wavelengths near the dye's emission peak and block all other light including the excitation light. Appropriate excitation and emission filter sets are use for each dye type.

Image analysis software to extract data from the images is essential in the microarrays of the current invention. This software preferably can identify array elements binding the fluorescent reporter, subtract background, decode multi-color images, flag or remove artifacts, verify that controls have performed properly, and normalize the signals.

Fluorescence Polarization Detection

Fluorescence polarization can follow the desorption of a member of a binding pair. In this assay type, a fluor-labeled binder is employed. The connection of the polarization with the desorption arises from the fact that Brownian motion, and consequently the magnitude of depolarization, occurring during the excitation lifetime, decreases as molecular size increases. Therefore, the desorption of a binding member causes a decrease in the polarization value because of the higher molecular weight of the binding pair over the individual members.

Fluorescence Resonance Energy Transfer (FRET) Assays

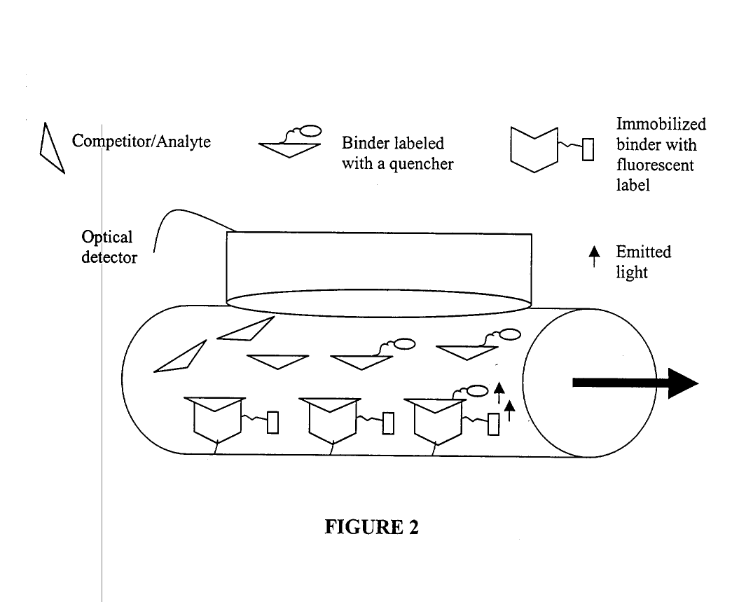

Fluorescence energy transfer is a process of energy transfer between two fluorophores, which can occur when the emission spectrum of the first fluorophore overlaps the absorption spectrum of the second fluorophore. Quenching of the emission from the first compound occurs, but the excitation energy is absorbed by the second compound, which then emits its own characteristic fluorescence. FIG. 2 illustrates an embodiment of this approach wherein the immobilized binding pair carries a quencher/emitter which emits light upon absorption of light emitted by the fluorophore attached to the other member of the binding pair. The emitted light is detected by an optical detector configured to receive the light from the immobilized binding pair. When a labeled binding pair member is desorbed the signal from the quencher emitter greatly decreases. Therefore, the presence of an analyte in the sample which competes with the binding of the labeled binding pair members causes a decrease in fluorescence of the fluorophore attached to the immobilized binding pair member. This change detects the presence of the competitor in a sample. Fluorescence resonance energy transfer (FRET) assays in spatially resolved chambers (e.g., microwells or microchannels) or on differentially labeled particles are envisioned for ultrasensitive and ultra-high throughput amino acid analysis in the current invention. The assay uses two labels, one of which is fluorescent donor and the other is an energy-accepting or energy-quenching molecule (acceptor). FRET assays detect binding in real time without a washing or separation step and are easily automated and miniaturized.

There are numerous recent reviews on FRET assays and many instruments for these assays are commercially available. Measurement of energy transfer is desirably based on fluorescence detection as this can provide high sensitivity. These assays and instruments are taught in (Clegg (1995) Curr. Opin. Biotechnology 6:103-110; Clegg. (1996) Fluorescence Resonance Energy Transfer (FRET) In: Fluorescence Spectroscopy and Microscopy, Wang, X. F., Hermann, B. (eds) J. Wiley and Sons, New York; Fultron et al. (1997) Clin. Chem. 43:1749-1756; Selvin, (1995) Methods Enzymol. 246:300-334; McDade (1997) Med. Dev. Diag. Indust. 19:75-82; Moerner et al. (1999) Science 283:1670-1676; Chen et al. (1999) Genet. Anal. 14:157-163; Mere et al. (1999) Drug Discov. Today 4:363-369.

Miniaturized Fluorescence Resonance Energy Transfer Assays

Miniaturized fluorescence resonance energy transfer (FRET) assays in spatially resolved microfluidic reaction chambers and microwells are envisioned for ultrasensitive and ultra-high throughput analysis in the current invention. FRET assays detect binding in real time without a washing or separation step, are easily automated and miniaturized and ultrasensitive. Successful applications of FRET are highly promoted by the introduction of modern instruments in fluorescence detection systems. The advantages of fluorescent lifetime imaging results from the fact that fluorescence lifetimes are usually independent of the fluorophore concentration, photobleaching, and other artifacts that affect fluorescence intensity measurements (Scully et al. (1997) Bioimaging 5:9-18). There are many reviews available on FRET and many instruments for these assays are commercially available (Clegg, R. M. (1995) Curr. Opin. Biotechnology 6:103-110; Clegg, R. M. (1996) Fluorescence Resonance Energy Transfer(FRET) In: Fluorescence Spectroscopy and Microscopy, Wang X. F., Hermann, B. (eds) J. Wiley and Sons, New York; Fultron et al. (1997) Clin. Chem. 43:1749-1756; Selvin, P. R. (1995) Methods Enzymol. 246:300-334; McDade, R. L.(1997) Med. Dev. Diag. Indust. 19:75-82; Moerner et al. (1999) Science 283:1670-1676; Chen et al.(1999) Genet. Anal. 14:157-163; Mere et al. (1999) Drug Discov. Today 4:363-369; Nie, S. and Zare, R. (1997) Annual Review of Biophysics and Biomolecular Structure 26:567-96). Spatially resolved fluorescence energy transfer has the capacity to detect, quantitatively, molecular interactions in real time over distances of microns.

Measurement of energy transfer is desirably based on fluorescence detection, thus ensuring high sensitivity. In addition to data acquisition with commercial microplate spectrophotometers, energy transfer methods can be incorporated into automated microfluidic assays for ultra-sensitive and ultra-high throughput analysis of biomolecular binding (Mere et al. (1999) Drug Discov. Today 4:363-369). The biomolecular interactions in the microwells can be monitored in all wells at the same time using a plate reader. Depending on the detectable tag used and the configuration, the plate reader can be a spectrophotometer, a fluorometer, a luminometer, a scintillation counter or a gamma counter.

Excitation is set at the wavelength of donor absorption, and the emission of donor is monitored. The emission wavelength of donor is selected such that no or very little contribution from acceptor fluorescence is observed. For instance, if a first binding pair member is labeled with fluorescein (fluor) and the second is labeled with rhodamine as described above, then fluorescein is the donor and rhodamine (Rh) is acceptor. Fluorescein excitation and emission wavelengths are around 490 nm and 520 nm, respectively. When both donor and acceptor labeled members are excited by monochromic light they fluoresce at different wavelengths. Fluorescence energy transfer between the binding member Fluor and the binding member-Rh is detected by measuring the photophysical properties of the donor fluorescence photons only. The acceptor photons may be barred from the detector by an optical filter; and therefore, the acceptor-labeled members that are not bound to the donor labeled members are not detected. Many donor/acceptor chromophores have been used in FRET assays and are suitable for use in the method of the present invention. For example, Wu et al. (1994) Anal. Biochem. 218, 1-13, lists 58 donor/acceptor pairs suitable for use in FRET assays.

Fluorescein measurements are carried out with the excitation at or around 490 nm and emission at 520 nm. Some fluorescent labels suitable for use in the subject invention include, but are not limited to, fluorescein (FITC, DTAF) (excitation maxima, 492 nm/emission maxima 516-525 nm); carboxy fluorescein (excitation maxima, 492 nm/emission maxima, 514-518 nm; 2=-methoxy-CF (excitation maxima, 500 nm/emission maxima, 534 nm); TRITC G (tetramethylrhodamine isothiocyanate, isomer G (excitation maxima, 535-545/emission maxima, 570-580); RBITC (rhodamine-B isothiocyanate (excitation maxima, 545-560/emission maxima, 585); Texas Red (excitation maxima, 595/emission maxima, 615-620); Cy-5 (Cyanine) (excitation maxima, 649/emission maxima, 670); Cy-3.5 (excitation maxima 581 nm/emission maxima, 596 nm); XRITC (rhodamine X isothiocyanate (excitation maxima, 582 nm/emission maxima, 601 nm); ethidium bromide (excitation maxima, 366 nm/emission maxima 600 nm); Thiazole orange (To-Pro) excitation maxima, 488 nm/emission maxima 530-580 nm).

Binding pair members can be site-specifically labeled. For instance, molecular biology methods such as site-directed mutagenesis and unnatural amino acid mutagenesis (Anthony-Cahill et al. (1989) Trends Biochem. Sci. 14:400) can be used to introduce cysteine and ketone handles for specific dye labeling of proteins (Cornish et al. (1994) Proc. Natl. Acad. Sci. USA 91: 2910-2914).

Imaging or scanning detectors including confocal scanners, charged coupled device arrays, photodiode arrays and optical fiber arrays can be used in the subject invention as reviewed in Brignac et al. (1999) IEEE Eng. Med. Biol. Mag. 18:120-22; Eggers et al. (1994) Biotechniques 17:516-525; Pang et al. (1999) J. Biochem. Biophys. Meth. 41:121-132; Setford et al. (2000) J. Chromatogr. A 867: 93-104; Kheterpal, I. and Mathies, R. A. (1999) Anal. Chem. 71:31A-37A; Crabtree et al. (2000) Electrophoresis 21:1329-35; Heiger et al. (1994) Electrophoresis 15:1234-1247; and Budach et al. (1999) Anal. Chem. 71:3347-3355.

Other Differential Detection Methods

Analytical methods based on competitive displacement of prebound binding pair member and employing multiple labels for the analysis of multiple amino acids in a sample is a further aspect of the current invention. By using multiple distinguishable labels, multiple discrete binding assays are performed in a single vessel at the same time. Multiple labels may be different fluorescent dyes, different radioisotopes, different dye or isotope ratios, different size particles, etc. The labels may be attached directly to the molecular recognition elements. Alternatively, the labels may be attached to a surface to which the molecular recognition elements are immobilized. Labels may be attached to proteins, nucleic acids, or other polymers for example. In some preferred embodiments of the current invention, uniquely distinguishable particles (e.g., microspheres, nanoparticles, metals, liposomes, vesicles, beads, proteins and the like) serve as labels for the binding pair members.

One known method for quantitative and simultaneous detection of multiple analytes in a sample is a flow microsphere binding assay (Reviewed in McHugh, 1994, Methods in Cell Biology 42: 575-595). This technique relies upon the ability of a flow cytometer to accurately detect different classes of microspheres based upon a physical characteristic such as size or color. The different microsphere classes are coated with different capture reagents and the fluorescence associated with each microsphere is quantitated with a flow cytometer.

For example, Luminex (Austin, Tex.) describe a method for encoding microspheres according to their fluorescence as taught in Fulton et al, 1997, Clin. Chem. 43:1749-1756 and U.S. Pat. No. 5,736,330 both of which are incorporated herein by reference. The methodology is based on the principle that fluorescent microspheres (beads) with unique fluorescent profiles can be immobilized to different analyte specific binders and used to create a fluorescence-based array of analyte specific beads where each bead type is specific for a unique analyte. This technology employs a combination of fluorescent dyes that allow each bead to be independently identified. The analyte specific microspheres are mixed together and contacted with a probe(s) that is labeled with a different fluorescent color. The probes bind to their ligands or receptors on the labeled microspheres and are used to determine the specific molecular interaction at the surface of each bead. The samples are read in a flow cytometer which allows each microsphere to be identified individually and the corresponding probe binding signal to be read. This technology has the potential to be faster, less expensive, and more sensitive than microarrays based on spatial separation.

The microspheres are available (Luminex, Austin, Tex.) in 64 distinct sets that are classified by virtue of the unique orange/red emission profile of each set. Different concentrations of each of two fluorochromes, orange-emitting and red-emitting, were used to prepare 64 microsphere sets with unique orange/red emission profiles. The microspheres can be covalently coupled to virtually any amine-containing molecule through surface carboxylate groups. Alternatively, avidin-coupled microspheres are available for immobilizing biotinylated molecules (Fulton et al, 1997, Clin. Chem. 43: 1749-1756).

The FlowMetrix™ system (Luminex, Corp) performs analysis of up to 64 different assays by using a flow cytometer. The flow cytometer analyzes individual microspheres by size and fluorescence. In this system three fluorescent colors, orange (585 nm), red (>650 nm) and green (530 nm), are simultaneously distinguished by the flow cytometer. Microsphere classification is determined by the orange and red florescence, whereas green fluorescence is used for labeling the probes. As each microsphere is analyzed by the detector, the microsphere is classified into its distinct analyte specific set (from the orange and red fluorescence) while simultaneously the green fluorescence on each bead is recorded. From this data, the identity and quantity of the multiple analytes are automatically determined. This technology has the potential to be faster, cheaper, and more sensitive than other array formats. For example, 512 different assays can be analyzed in a single well in a few seconds (Chandler et al, 1998, Cytometry Suppl 9:40).

Michael et al., (1998) Anal. Chem. 70:1242-1248 teach a method of multianalyte analysis where mixtures of different microspheres, each a different assay, are applied to an optical sensor array for detection. Single microspheres immobilized in wells etched from optical fiber bundles have the potential for array elements to be in the sub-micrometer size range. Each different microsphere is tagged with a unique combination of fluorescent dyes. This optical labeling technique is simply a combination of fluorescent dyes with different excitation and emission wavelengths and intensities that allow each bead to be independently identified. This type of labeling is similar to that used by Luminex in its multiplexed flow cytometer arrays. The optically labeled arrays can be decoded in a matter of seconds with conventional image processing software by collecting a series of fluorescent images at different excitation and emission intensities of each unique bead. Excitation light is launched into the fiber. Light emitted form the fluorescent dyes on the fiber's distal tip is carried back along the fiber and filtered before image capture on a CCD camera. Optical fiber arrays offer rapid, multiplexed, and sensitive detection(absolute detection limits of zeptomole, 10⁻²¹ moles of DNA. See, Walt (2000) Science 287: 451-452); and Walt et al. U.S. Pat. No. 6,023,540 which are each herein incorporated by reference.

Bead assays have recently become popular, for example, for gene expression analysis by massively paralleled signature sequencing on microbead arrays, see Brenner et al. (2000) Nature Biotechnology 18: 630-634; surface plasmon resonance binding assays, Lyon et al.(1998) Anal. Chem. 70: 5177; DNA colorimetric nanoparticle assay, Storhoff et al. (1998) J. Am. Chem. Soc. 120, 1959, and solution based DNA hybridization, Elghanian et al.(1997) Science 277: 1078.

The microarrays, microsystems, or kits of the present invention can be readily incorporated into the technologies of the current art. The binders of the subject invention may be immobilized in any number of ways. The methods for array construction or biomolecule immobilization are not important in the subject invention, as a vast number of methods known in the art are suitable.

Many types of microplate fluorometers are commercially available. Formats using fluorescent labels and microplate fluorometers enable femtomole-attomole sensitivities. Molecular Device's FLIPR or LJJ Biosystem's new Acquest have the ability to handle 1536-well plates and have a high degree of automation. Bio-Tek Instruments' Model FL600 microplate fluorometer can detect less than 2 femtomoles of fluorescein with a read time of 28 sec. Molecular Device's SPECTRAmax Gemini microplate fluorometer can detect 5.0 femtomoles of FITC in 96-well plates with a read time of less than 27 sec, and BMG Lab Technologies' FluoStar can detect 50 attomoles/well Eu3⁺ reading 384 wells in 30 sec. Instruments are also available that combine time-resolved fluorescence with fluorescence resonance energy transfer pairing. This combination requires two fluorophores emitting at different wavelengths. The first emits right away, but the second is activated only when the two are in proximity, i.e., when two labeled molecules are bound. This allows simultaneous measurement of bound and unbound analytes and thus permits internal calibration. It also means that the assay is homogenous, and therefore, it is easy to automate and miniaturize.

Antibody Techniques

Monoclonal or polyclonal antibodies, preferably monoclonal, specifically reacting with a particular binder member of interest may be made by methods known in the art. Also engineered antibodies and antibody binding fragments can be employed. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York, and Ausubel et al. (1992) Current Protocols in Molecular Biology, Green Wiley Interscience, New York, N.Y.;

DNA Technology

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed) (1993) Meth. Enzymol. 218, Part In; Wu (ed) (1979) Meth. Enzymol. 68; Wu et al. (eds) (1983) Meth. Enzymol. 100 and 101; Grossman and Moldave (eds) Meth. Enzymol. 65; Miller (ed) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed) (1985) DNA Cloning Vol. In and II, IRL Press, Oxford, UK; Hames and Higgins (eds) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley Interscience, New York, N.Y. Abbreviations and nomenclature, where employed, are deemed standard in the field and commonly used in professional journals such as those cited herein.

Temperature Control Technology

Microflow PCR methods rely heavily on temperature of a fluidic environment and such temperature control methods are readily adaptable to the present systems. See, for instance, the temperature control systems described in the following references:

Lagally E T, Medintz, and Mathies R A (2001) “Single-molecule DNA amplification and analysis in an integrated microfluidic device” Anal Chem 73, 565-70. This reference teaches a method using thin film heaters which permit temperature cycle times as fast as 30 seconds. At least 3 different temperatures are used.

Giordano B C, Ferrance j, Swedberg S, Huhmer A F, and Landers, J P (2001) “Polymerase chain reaction in polymeric microchips: DNA amplification in less than 240 seconds” teach a method using infrared-mediated temperature control to accurately thermocylcl microliter volumes in microchips fabricated from polyimide.

Khandurina J et al (2000) “Integrated system for rapid PCR-based DNA analysis in microfluidic devices” Anal Chem 72; 2995-3000. They teach a method of temperature change and control using dual Peltier thermoelectric elements.

Huber M et al (2001) “Detection for single base alterations in genomic DNA by solid phase polymerase chain reactions on oligonucleotide microarrays”

Woolley, A T et al (1996) Anal Chem 68, 4081-6 “Functional integration of PCR amplification and capillary electrophoresis in a microfabricated DNA analysis device” teach methods for changing temperatures in a microfabricated device.

Belgrader P. et al. (2001) “A battery-powered notebook thermal cycler for rapid multiplexed real-time PCR analysis” miniaturized heaters are integrated into the device and independent control of the heaters allows for differing temperature profiles and detection schemes to be run simultaneously.

J. M. Ramsey and A. van den Berg (eds.), Micro Total Analysis Systems 2001 (Kluwer Academic Publishers, Dordrecht, Boston, London) see therein in particular the following articles:

E. Lagally and R. Mathies “Integrated PCR-CE System for DNA Analysis to the Single Molecule Limit”, pp. 117-118.

C. F. Chou et al. “A Miniaturized Cyclic PCR Device”, pp151-152.

Chiou et al. “Performance of a Closed-Cycle Capillary Polymerase Chain Reaction Machine”, pp. 495-496.

Miniaturized pH Detection of Microchips

The miniaturized detection of pH has been described in the prior art as well. See, for instance, the following references: Tantra R, Manz A (2000) “Integrated potentiometric detector for use in chip-based flow cells. Anal Chem 72, 2875-8; Cui Y, Wei Q, Park H, Lieber C M (2001) “Nanowire nanosensors for highly sensitive and selective detection of biological chemical species” Science 293, 1289, and Grant, S A et al (2000) “Development of fiber optic and electrochemical pH sensors to monitor brain tissue” Crit Rev Biomed Eng 28, 159-63.

### EXAMPLES

The following examples are provided for illustrative purposes, and are not intended to limit the scope of the invention as claimed herein. Any variations in the exemplified articles and/or methods which occur to the skilled artisan are intended to fall within the scope of the present invention.

### Example 1

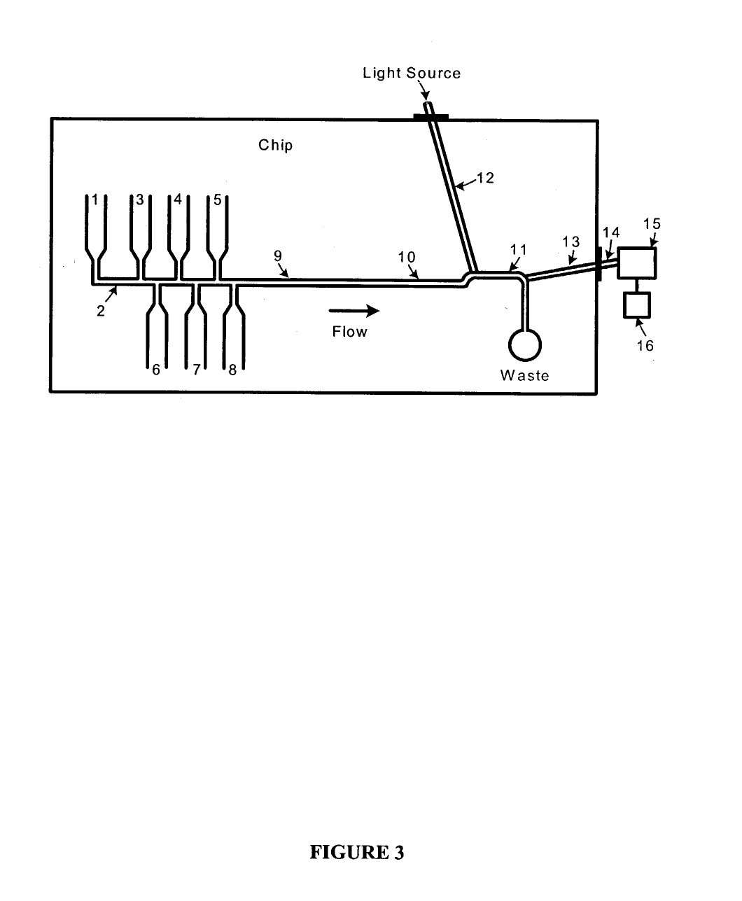

FIG. 3 is a schematic drawing of a continuous microflow system employing biospecific desorption and optical detection of the desorbed binder. The chip is preferably constructed in two parts comprising a base part and a lid part. The body of the microfluidic chip includes a first planar substrate that is fabricated with a series of groves and/or depressions in its upper surface. The grooves or depressions correspond to the channel/chamber geometry of the finished device. A second planar substrate (e.g. pyrex) is then overlaid and its lower surface is bonded to the surface of the first substrate to seal and define the channels of the device. Ports/reservoirs are provided in the body structure and in fluid communication with the channels of the device. The reservoirs or ports are generally constructed as apertures disposed through the upper substrate layer. These holes connect the upper surface with the lower surface of the lid and are in fluid communication with one or more of the sealed channels. The devices include an optical detection window to permit measurements of optical signals from the channel. Microfluidic devices incorporating this planar body structure with optical detectors are well known in the prior art.

Buffer flows through microchannel 2 from buffer reservoir 1 by virtue of a micro fluidic transport mechanism. For example, a pneumatic micro fluidic pump or an electroosmotic pump may be employed. Such microfluidic pumps are well known in the prior art. A plurality of reservoirs or sample ports (only 7 are shown in the figure, 1 and 3-8) connect to microchannel 2, allowing a liquid sample to be introduced into microchannel 2 from each port or reservoir one at a time. Downstream from the sample ports or reservoirs starting at point 9 the microchannel has, immobilized (e.g. by covalent attachment or noncovalent attachment by avidin-biotin binding) a binder that has its binding sites bound with labeled (e.g. fluorescently labeled) cognate binding partner. Binders may be immobilized within the reaction chamber by binding them to the inner walls of the channel or to suitable solid supports. Suitable solid supports include those that are well known in the prior art, e.g. agarose, cellulose, silica, polystyrene, etc. Further downstream at point 10 the immobilized binders terminate. Downstream of point 10 is a detection cell 11. Chip detection cells are known in the prior art. For example, a chip cuvette is disclosed in Liang, Z et al. (1996) Analytical Chemistry, 68:1040-1046. The detection cuvette includes at least one window transparent to excitation light and one window transparent to fluorescent emissions. Optical fiber 12 transports excitation light to detection cell 11. The excitation light causes any biospecifically desorbed or displaced fluorescently labeled binders to emit fluorescent light.

Excitation wavelengths and light sources may depend on the fluorescent labels used. For example if fluorescein is used as a label an argon ion laser may be employed with excitation at around 488 nm with an emission peak at around 520 nm. If Cy5 is used as a label excitation is at around 649 nm and emission is at around 670 nm. The light source in this case may be a HeNe laser, or a diode laser.

An additional optical fiber may be employed to transport fluorescent light to a detector. Alternatively, the light may be transported form a source to the detector cell and back to the detector through the same fiber. Methods for delivering excitation light to microchannels and for receiving emission light to detectors are well known in the prior art. Optical fiber 13 transports fluorescent emission light to detector 15 through a coupler and optical fiber 14. The detector 15 is linked to a computer 16 that may programmed for data analysis.

Appropriate filters for excitation light and fluorescent emissions may be added at any points along the light paths. For example, filters may be incorporated into the ends of detector cell 11 between the light source and optical fiber 12 and/or between detector 15 and optical fiber 14.

In another embodiment the light sources, detectors, and filters may be incorporated into the chip. Data from a detector (e.g. a photodiode or photomultiplier tube) within the chip can be ported to a computer via, for example, an RS232 port built into the chip. The circuitry for each of these components may be provided on the chip. Examples of optical elements that may be fabricated into or attached to the body structure include lenses, optical filters, optical gratings, beam splitters, waveguides, TIR mirrors, lasers, polarizers and the like. For a discussion of these optical elements integrated into chips see e.g Handbook of Optics, Vol II (1995) McGraw-Hill.

### Example 2

In one preferred embodiment of FIG. 1, a continuous microflow system uses a displacement assay that measures the fluorescent signal of a displaced labeled binder analogous to the analyte binder. A known density of an immobilized molecule (e.g., antibody, antibody fragment, protein, peptide, carbohydrate, lipid, cell, cell fragment, organelle, nucleic acid, dye, inhibitor, receptor, and the like) that specifically and reversibly binds the analyte binder and labeled analyte binder analog is immobilized in a buffer flow and saturated with a fluorophore-labeled cognate binder. Introduction of the analogous unlabeled analyte binder (for example, a receptor on a cell surface, a functional motif or domain in a protein) results in a proportionate displacement of its analogous bound labeled binder. The displaced labeled binders are carried from the sites bearing the immobilized capture elements by mass transfer (e.g. by flowing buffer) and detected downstream by a detector. This displacement may occur within seconds of exposure to the unlabeled analyte binder. Standard curves using known concentrations of unlabeled analyte binder may be established. Also, displacement efficiencies may be established using known antigen concentrations.

The biorecognition elements may be immobilized on any surface to be contacted with a sample. For example, the recognition elements may be immobilized on the surfaces or transducers including optical fibers and microelectrodes. In some preferred embodiments, the binders may be immobilized on beads or nanoparticles and placed in a flow channel. Alternatively, the binders may be immobilized to the surface of the microchannels. In these cases, the biospecific desorption of the labeled analyte analog may result in a proportionate decrease in signal at the transducer surface. The optical fibers or microelectrode arrays may be placed in a flow stream. The labeled analyte (using for example fluorescent or electrogenic labels for optical fibers or microelectrode respectively) may be biospecifically desorbed resulting in a decrease in signal thereby providing the means for detecting the biospecific interaction.

### Example 3

In preferred embodiments, methods, systems and apparatus according to the present invention are applied to the analysis of amino acid samples by competitive displacement of binding pair members wherein one of the members is an amino acid. U.S. patent application Ser. No. 09/927,424 filed Aug. 9, 2001 and assigned to the same assignee and incorporated herein by reference teaches suitable microflow systems and binding member pairs for conducting such studies. For instance, elongation factor IA or Tu:GTP can serve as a biorecognition element for an aminoacyl-tRNA.

### Example 4

Cell adhesion molecules have been recognized to play a major role in a variety of physiological and pathological phenomena. They determine the specificity of cell-cell binding and the interactions between cells and extracellular matrix proteins. The receptors that mediate adhesion between cells that may be studied in flow systems invented herein include integrins, selectins, the immunoglobulin superfamily members and cadherins. Ligand binding characteristics of these adhesion molecules may be studied in these systems.

For instance, the current invention can be applied to studying the binding of cellular adhesion proteins and other proteins to extracellular matrix proteins and domains or fragments thereof and in screening for inhibitors of such specific binding. “Extracellular matrix proteins” which may be used as binders include the following: aggrecan, argin, bamacan, BEHAB, biglycan, bone sialoprotein, brevican, cartilage matrix protein, chondroadherin, collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, decorin, dentine matrix protein, dentine sialoprotein, elastin, fibrillin I, fibrillin-2, fibrinogen, fibromodulin, fibrinonectin, fibulin-1, fibulin-2, keratocan, laminins, latent transforming growth factor beta binding protein-1, latent transforming growth factor beta binding protein-2, latent transforming growth factor beta binding protein-3, link protein, lumicin, lysyl oxidasematrix gla protein, microfibril-associated glycoprotein-1, microfibril-associated glycoprotein-2, MMP1, MMP2, MMP3, neurocannidogen, osteocalcin, osteonectin, osteopontin, perlecan, phosphophoryn, procollagen C-proteinase, procollagen I N-proteinase, tenascin-C, tenascin R, tenascin X, tenascin Y, thrombospondin-1, thrombospondin-2, thrombospondin-3, thrombospoondin-4, versican, vitronectin, von Mayibrand factor, thrombin, plasminand others.

### Example 5

The current invention has applications in studying the cell adhesion and cell contact regarding cell-cell and cell-extracellular matrix adhesions and inhibitors of such adhesions. Cell adhesion and cell-cell contact proteins relevant to the subject which can be used as binders include the following proteins or fragments or domains thereof and others: The Ig superfamily of adhesion molecules, cadherins, integrins, CCAMs (cell-cell adhesion molecules), CD2, LFA-3, CD44, cells surface glactosyltransferase, chemokine receptors, c-kit receptor tyrosine kinase-kit ligand/stem cell factor, connections, contact site A, DCC family, dystroglycan, beta. 3-endonexin, Ep-CAM (epithelial cell adhesion molecule), fasciclin I, fasciclin II, fasciclin III, intigrin-associated proteins, ICAMs, glypicans, leucine-rich repeat family, LFA-1, MAdCAM-1, mannose binding protein (MBP), MHC class I and II, MEG (myelin associated glycoprotein), MBPs (myclin basic proteins), MOG (myelin oligodendrocyte glycoprotein), peripheral myelin protein 22 (PMP22), protein zero (Po), NCAM (neural cell adhesion molecules), neural cell recognition molecule F11 (contactin), neural cell recognition molecule L1, neurofascin, neurotactin, notch/delta/serrate, NgCAM-related cell adhesion molecule (NrCAM), occludin, PECAM-1//CD31, PH-20, platelet GP Ib-IX-V complex, selecting, E-selectin, L-selectin, P-selectin, CD34, snyndecans, TCR/CD3 complexes and the CD4 and CD8 co-receptors, UNC-5 family, VCAM-1.

### Example 6

Examples of domains of adhesion or extracellular matrix molecules suitable for use as binders in the subject invention include fibrinectin type I domain, fibrinonectin type II domain, fibrinonectin type III domain, fibrinogen gamma C-terminal domain, kunitz-type inhibitor domain, immunoglobulin domain, receptor class A domain, low density lipoprotein domain, laminin N-terminal domain VI, epiderminal growth factor like domain, extracellular calcium-binding domain, collagen IV C-terminal domain, collagin I C-terminal domain, cadherin extracellular domain, C-type lectin domain, endostatin domain in collagen type XVIII, compliment control protein/short consensus repeat/Sushi domain, gamma-carboxyglutamate domain, haemopexin domain, link hyalluronate binding domain, argin/perlecan/enterokinase domain, somatomedin B domain, thrombospondin type I/properdin domain, thrombospondin type 3 calcium-binding domain, von Mayebrand factor type A domain, von Mayebrand factor type B domain, leucine rich repeat domain, serine/threonine-rich domain.

### Example 7

Many different cell surface molecules can serve as binders for the attachment of viruses. These cell surface molecules include, but are not limited to, heparin sulphate, Vcaml, CD55, sialic acid, Icam-1, low-density lipoprotein family, aminopeptidase N, high-affinity laminin receptor, alpha-dystoglycan, integrins, CD4, epidermal growth factor receptor, vitronectin receptor, HAVCr-1

### Example 8

The biospecific desorption microsystems may be used with antibodies as members of a binding pair. The systems can be used particularly for high throughput screening of monoclonal antibodies to obtain those with suitable binding characteristics to be used for affinity purification of proteins or other molecules. Monoclonal antibodies are routinely used to affinity purify proteins and other molecules. However, the antibody must bind the analyte tightly enough so that it may be retained during washing yet the dissociation constant must be suitable for elution of the purified molecule in an active form. Binding characteristics suitable for affinity screening of antibodies can be determined in microsystems described below. These automated microsystems can screen hundreds to thousands of antibodies simultaneously using tiny amounts of reagents. In one embodiment, such a system would include the following features:

1. A different monoclonal antibody to the analyte is immobilized in each microchannel in an array of channels.

2. Each immobilized antibody is saturated with a labeled analyte analog.

3. The analyte is flowed through the microchannel array at different concentrations.

4. The labeled analyte analogs may be biospecifically eluted by the analyte. The concentration of the analyte that causes this elution may depend on the dissociation constant of the immobilized antibody. From the concentration and time required to cause a proportionate displacement, the dissociation rate constant may be computed.

5. The labeled analyte analogs may be biospecifically eluted by the analyte. The concentration of the analyte that causes this elution may depend on the dissociation constant of the immobilized antibody. From the concentration and time required to cause a proportionate displacement, the dissociation rate constant may be computed.

6. These competitive displacement microsystems may be used to select monoclonal antibodies and other binders having dissociation constants suitable for measuring binding in the continuous elution microsystems invented herein.

### Example 9

An integrated competitive displacement microfluidic system for the simultaneous analysis of multiple functional elements is also envisioned in which a unique labeled binder analyte analog may be immobilized to its cognate capture element in an array of such elements. Using microfluidic arrays, each microchannel in the array may have a different labeled analyte analog bound to its cognate immobilized capture element. The sample may flow from a main microflow channel into the micro fluidic array. As the sample inters the array through inlets, it may displace labeled analyte analogous only in microchannels having an immobilized labeled analyte analogous to that present in the sample. From the spatially specific detection of the entire array of microchannels, it may be possible to determine which analyte analogous are present in the sample. For example, see FIG. 2. The labeled molecules may be displaced and flow past the array detectors and be continuously identified.

### Example 10

The competitive displacement inventive methods and devices can be used for a binding pair member or ligand for to obtain a dissociation constant even if it binds too loosely to its binding partner or receptor to perform a direct binding experiment. Most of the physiological neurotransmitters and hormones bind to their receptors with affinities in the 0.1-1.0 micromolar concentration range. In these cases displacement or competition experiments are the methods of choice. For these experiments one can compute the dissociation constants. This displacement of the labeled ligand by non-labeled competition is monitored and the dissociation constant for the nonlabeled ligand can be computed.

### Example 11

In one embodiment, cancer-specific cellular receptors are used as a binding pair member. In further embodiments, the cancer-specific cellular receptor is the immobilized member of a binding pair. Novel potential binding pair members include growth factor receptor tyrosine kinases such as epidermal growth factor receptor and HER-2/neu (proliferation) and the vascular endothelial growth factor receptor and the basic fibroblast growth factor receptor (angiogenesis).

### Example 12

In some embodiments, the competitive displacement methods and devices of the present invention are applied to study of the functional domains of proteins and polypeptides. The methods are particularly useful in determining the functions and properties of proteins and polypeptide fragments and other biopolymers such ribozymes identified only from corresponding polynucleotides sequences. Genome projects are currently producing many thousands of gene sequences from which protein amino acid sequences may be deduced. From these data putative binding sites may be identified based on consensus sequences. The relationship between genotype and phenotype is far too complex to be predicted form genomic sequence data; hence, proteins and must be studied directly. Most amino acid residues in a protein are stabilizing elements and only a small percentage participate directly as binding sites. The active binding patches on a protein's surface are created by specific amino acid sequences and function as specific adsorption patches. It is desirable to map these binding patches for all proteins. In this way we may obtain an understanding of biology and pathology at the molecular level and rational drug design may be possible. The current art has not developed a method for rapidly mapping binding sites on proteins, nucleic acids, or other biopolymers. Microflow systems are invented herein for rapidly identifying specific binding sites on the surfaces of proteins, nucleic acids, and other biopolymers.

Computer controlled and integrated microflow systems suitable for automated analysis of biospecific interactions with on-line high throughput screening for inhibitors of biospecific interactions are disclosed herein.

Proteins are molecular mosaics composed of a wide variety of conserved sequence motifs. As entire genomes of organisms are sequenced, the open reading frames allow the amino acid sequences of all potential proteins to be established. One extremely effective method for the characterization of a newly discovered protein involves the comparison of its amino acid sequence (as predicted from the genome sequence) with the sequences of previously characterized proteins having known functions. The rapid increase in the accumulation of sequence data from genome programs has made database searching routine and mandatory.

Sequence comparison methods enable the search for functional motifs in proteins and for sites of covalent modification. This has become established as a “first approximational” aid to the study of purified proteins of unknown function.

It is not sufficient, however, to determine the primary structure (amino acid sequence) of a protein or deduce it from the DNA sequence and expect that this may reveal all or any of the functions of a protein. The conservation of putative functional elements, that is, consensus sequences, does not ensure a function. The conservation of functional sequence elements varies some being highly conserved while others permit substitutions and remain functional. In many cases, the sequence motif may be highly conserved and yet nonfunctional. Consensus sequence information functions only as a guide. All of the many thousands of consensus sequences arising from genome programs must confirmed or refuted experimentally. The chemical nature and positions of all functional motifs and modifications of a protein that are necessary for its correct action, regulation, and antigenicity must be established by experimentation.

The methods invented herein may provide a means for ultra high throughput analysis of putative functional elements (specific binding) arising from genome programs.

### Example 13

Competitive displacement microflow systems according to the invention may be directed toward determining the presence of functional domains or motifs within a protein, polypeptide, domain, or protein fragment. These embodiments, typically would involve immobilization of the binding pair member in a microchannel. Consensus sequences have been defined for many of the known post-translational modifications, signal sequences, and functional domains in addition to functional motifs. This information may suggest a function (e.g. binding a specific molecule) for a previously uncharacterized protein. Ultra-high throughput methods to confirm or refute consensus sequence information resulting from genome programs are needed. The methods and systems invented herein provide such a technology. Microflow systems are invented herein to allow the automated screening of the presence of co- and post-translational modifications and to establish whether or not consensus sequence derived putative binding sites actually bind their putative ligands. Antibodies and other ligands that reversibly and specifically bind co- and post-translational modification sites are used in the microflow systems invented herein where biospecific desorption is employed to identify co- and post-translational modifications on proteins.

### Example 14

Components of the extracellular matrix may be adsorbed in microflow channels and their ligand binding characteristics may be studied as outlined in claim 1. Examples of extracellular matrix components that may be immobilized in the flow channels include proteoglycans, or fragments thereof, hyaluronan (hyaluronic acid), heparin sulphate heparins, chondroitin sulphate, dermatin sulphate, keratin sulphate, glycoproteins or fragments thereof. (Specific examples of such proteins include fibronectins, laminin, thyrombospondin, von Mayebrand factor, osteoponiin, bone sialoprotein, fibrillin MAGP, aggrecan, argon, bamacan, BEHAB, Biglycan, bone sialoprotein, brevican, cartilage matrix protein, chondroadherin, collagin type I, collagen type II, Collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, hydroxyapatite, collagen type XIX, decorin, dentine matrix protein, dentine sialoprotein, elastin, fibrillin-1, fibrillin-2, fibrinogen, fibromodulin, fibronectin, fibulin-1, fibulin-2, keratocan, laminins, latent transforming growth factor-beta binding protein-1, latent transforming growth factor-beta binding protein-2, latent transforming growth factor-beta binding protein-3, link protein, lumican, lysyl oxidase, matrix Gla protein, microfibril-associated gylcoprotein-1, microfibril-associated glycoprotein-2, microfibril-associated glycoprotein-3, MMPI, MMP2, MMP3, MMP7, MMP8, MMP9, MMP10, MMP11, MMP 12, MMP 13, Neurocan, Nidogen, osteocalcin, osteonectin, osteopontin, perlecan, phosphophoryn, PRELP, procollagen c-proteinase, procollagen I N-proteinase, tenascin C, tenascin Y, tenascin X, tenascin R, thrombospondin-1, thrombospondin-2, thrombospondin-3, thrombospondin-4, thrombospondin-5, TIMPI, TIMP2, TIMP3, versican, vitronectin, The ability to influence cell behavior by allowing attachment and migration of cells may be studied in the micro flow systems as outlined in claim 1.

### Example 15

In one embodiment, a binding member of the invention is collagen. The collagens constitute a highly specialized family of glycoproteins of which there are at least 19 genetically distinct types encoded by 34 genes.

### Example 16

In another embodiment, the micro flow system binding pair member or ligand is a cell membrane immobilized in a flow channel. Labeled analyte analogues are perfused through the system or allowed to bind to the membranes. Soluble receptors, cells, or fragments are perfused through the system. The presence and quantity of the receptor binding the labeled molecule may be detected as the labeled molecule is biospecifically eluted or captured. The labeled molecule may either flow past a detector or be detected as a decrease in signal if the detector monitors the immobilized labeled ligand.

### Example 17

In another embodiment, the immobilized binding pair member comprises an extracellular matrix immobilized in a micro flow channel.

### Example 18

In other embodiments, a micro flow system has cells as the immobilized binding pair in fluidic contact with the microflow channels. These cells may mimic tissues, organs, or blood vessels. Endothelial cells may be immobilized in microflow channels and may thereby mimic the blood walls. Blood coagulation may be studied in micro flow systems. This may be accomplished by determining the rate of flow continuously in the presence of proteins and other substances (e.g. platelets, heparins, lipids, drugs). The formation of a clot and the by continuously monitoring the flow rates with in the microflow systems. Substances influencing blood coagulation may be pre fused through the microchannels.

### Example 19

In another embodiment, a micro flow system is provided wherein fibrinogen is the binding pair member to be immobilized in the microflow system. Fibrinogen is the protein forming the blood clot. Fibrinogen may be converted into fibrin forming a blood clot within the microflow system subjected to drugs, proteins, lipids, and other substances. Clot lysis may be studied in an automated micro system. Potential fibrinolysis causing substances may be perfused through the system automatically and the flow rate or detection of lysis products may be used to identify substances causing fibrinolysis.

### Example 20

In another embodiment, a bone matrix is adsorbed in the microchannel and functions as an immobilized binding pair member would. Bone resorption is a medically important phenomenon that may be studied in microflow systems. Substances that prevent bone resorption may be identified. Osteoclasts and osteoblasts may be studied in these systems. Substances that cause bone deposition and resorption may be identified. Protein, protein-lipid, protein-carbohydrate, interactions systems can be studies using biospecific desorption.

### Example 21

In another embodiment of the competitive displacement microflow systems of the invention, a binding pair member is an antibody or oligonucleotide aptamer that has been immobilized in a microchannel. These members can be obtained that detect virtually any substance with high specificity. Antibodies that specifically bind phosphorylated amino acid residues are commercially available and may be used in these micro flow systems to detect phosphorylated amino acids. In like manner, antibodies, olignucleotide aptamers, or fragments thereof may be used to detect other co- and post-translational modifications, affinity tags, conformational elements, domains and motifs.

### Example 22

In another embodiment, a competitive displacement micro flow system is used to study osteoblast adhesion on biomaterials. The proteins involved in osteoblast adhesion that may be immobilized as binding pair members in these flow systems include, but are not limited to, extracellular matrix proteins, cytoskeleton proteins, integrins, cadharins, cartilage matrix protein, matrix metalloproteinases. These flow systems may particularly find use in the field of tissue engineering in the field of orthopedic surgery. Two fields of research in particular are emerging: the association of osteogenic stem cells with these materials (hybrid materials). In both cases, an understanding of the phenomena of cell adhesion and in particular, understanding of the proteins involved in osteoblast adhesion on contact with the materials is of crucial importance. Any of the proteins involved in osteoblast adhesion may be studied in the automated micro flow systems invented herein.

### Example 23

A competitive displacement micro system for studying protein-protein, protein-carbohydrate, and protein-lipid interactions for proteins involved in the blood coagulation cascade is also envisioned. Specific proteins to be immobilized within the micro flow system include fibrinogen, prothrombin, thrombin, factor V, factor VII, factor VIII, factor, IX, factor X, factor XI, factor XII, factor XIII, protein C, protein S, protein Z, prekallikrein, HK, fibronectin, antithrombin III, plasminogen, urokinase, thrombin receptor, plasminogen receptor, urokinase receptor, protein C receptor, factor V receptor, heparin cofactor 11, heparin, alpha2-macroglobulin, protein C inhibitor, TAFI, alpha2 antiplasmin, thromodulin, platelets, platelet membranes, endothelial cells, endothelial cell membranes, lipoproteins.

### Example 24

A competitive displacement micro flow system is also envisioned for determination of protein-carbohydrate interactions. These micro flow systems may focus in particular on lectins. The initial contact formation between leukocytes and activated endothelium makes use of selecting to guide lymphocyte trafficking. Animal lectins are involved in cell-cell and cell-matrix interactions. The microsystems invented herein may provide a means for rapid and automated screening approaches for inhibitors to these interactions.

### Example 25

A micro flow system is also envisioned for the study of fibrinolysis. Proteins and or cells involved in fibrinolysis may be immobilized in micro flow channel(s). Fibrinolysis is essential for maintaining the fluency of blood flow. Attenuated fibrinolytic activity has been frequently detected in coronary artery disease, peripheral vascular disease, diabetes, hyperlipidemia and obesity. The biologically active product of the fibrinolytic system is plasmin. Generation of plasmin is regulated by plasminogen activators (PA) and their inhibitors (PAI). Vascular endothelial cells and smooth muscle cells synthesize tissue-type and urokinase-type PA (tPA and uPA) and their major physiological inhibitor, PAI. The production of fibrinolytic regulators is modulated by a number of biological factors related to thrombosis and atherosclerosis, including but not limited to coagulation factors, hormones, growth factors, inflammatory mediators and lipoproteins. In addition, several anticoagulants, including heparin, hirudin and hirulog-1, affect the production of fibrinolytic regulators in vascular cells. In addition to measuring the binding of specific ligands to cells, cell fragments, proteins, carbohydrates, lipids, and drugs to components of the fibrinolytic system, micro flow systems are envisaged where by the integrity of the clot (i.e., fibrinolysis) may be determined on line continuously in a micro flow system. This may be achieved by monitoring the flow rate through the clot or by optical detection of changes in the clot in response to clot forming or clot dissolution.

### Example 26

In another embodiment, the competitive displacement microsystems are directed toward studying the interaction of platelets to the subendothelium. The adhesion of circulating platelets to the sub endothelium is mediated by glycoprotein (GP) residing on the cell's surface. GPIIb/IIIa is the most important platelet membrane receptor that mediates the process of platelet aggregation, and thrombus formation. Thus, new drugs that block the GPIIb/IIIa receptor are needed. In the micro system claimed, platelets, platelet membranes, endothelial cells, membranes, or cell fragments or receptors and other proteins from these cells or platelets involved in platelet-endothelial cell interactions may be immobilized in micro flow channels. One of the binding partners which is reversible bound may be labeled. Drugs or other substances that cause the desorption of the binding pair may be perfused through the system. Drugs that inhibit binding may cause a desorption of the labeled binder and may be detected as the eluted labeled analyte is detected.

### Example 27

In another embodiment, the micro system is configured to study protein-vascular cell interactions. Vascular cells such as endothelial cells, smooth muscle cells, macrophages, neutrophils, platelets, and monocytes or fragments thereof may be immobilized in micro flow channels. Labeled analytes may be reversibly bound to the cell or membrane surface. Inhibitors to this biospecific interaction maybe perfused through the microflow channels. Inhibitors may be identified and characterized by flowing the signal form the labeled eluted binder as described in claim 1.

### Example 28

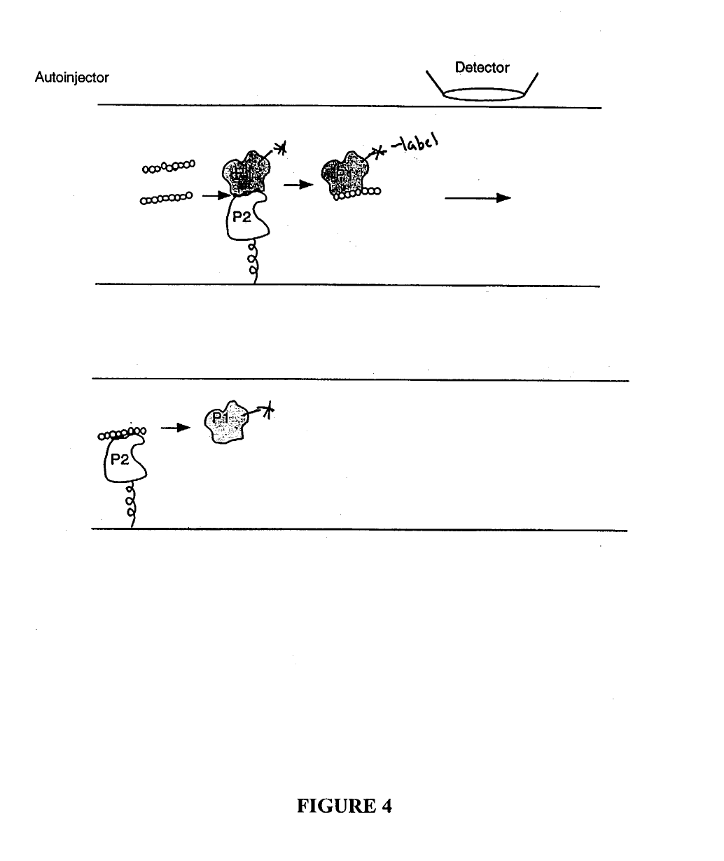

FIG. 4 illustrates the use of the inventive methods and microflow systems to study protein-protein interactions. Overlapping synthetic peptides can be made corresponding to the amino acid sequences of the interacting proteins. One of the proteins can be immobilized and the other can be allowed to bind. A series of synthetic peptides can be injected into the flow chamber by an auto injector. When peptides corresponding to the binding sites of the proteins are injected, the bound protein can be eluted and detected by the detector. The detector and autoinjectors can be integrated. A computer-controlled system (not shown) can have a record of which peptide was injected causing the elution, and hence an automated system for mapping binding sites on protein surfaces is embodied.

### Example 29

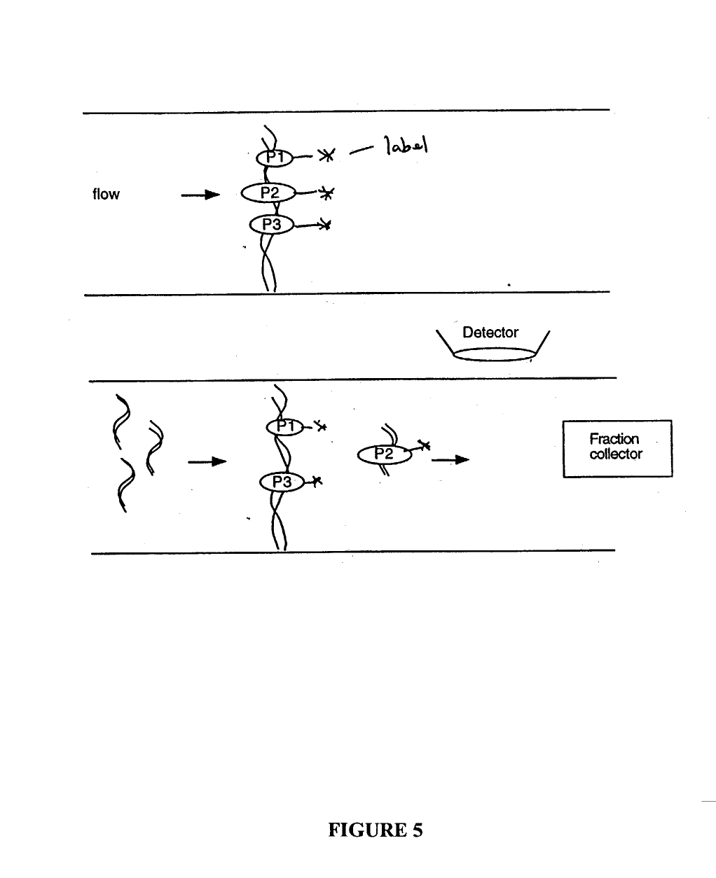

FIG. 5 illustrates the use of the inventive methods and microflow systems to study protein-nucleic acid interactions-rapid promoter analysis. In FIG. 5, a promoter is immobilized in a flow chamber and the chamber is perfused with a cell extract. Next, the flow chamber is perfused with a wash buffer. After the wash step, the chamber is perfused with a series of overlapping double stranded oligonucleotides corresponding to the sequence of the immobilized promoter. As the oligonucleotides corresponding to the DNA binding site on the bound proteins flow through the chamber, the proteins are eluted, detected by the detector, and collected by a fraction collector. All steps are automated and computer-controlled. The peptides are added by an autoinjector. The autoinjectors, detectors, and fraction collectors are all integrated. Thus, the promoter binding proteins are purified and the DNA binding sites identified simultaneously. The eluted proteins can then be subjected to microanalysis. Bound proteins may be derivitized on line with fluorescent labels for ultrasensitive detection.

### Example 30

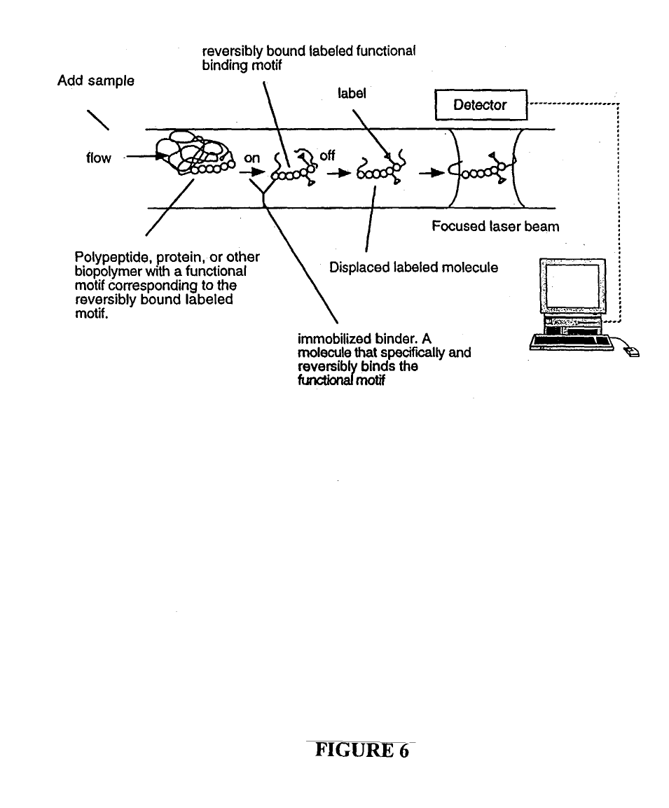

FIG. 6 illustrates the use of the inventive methods and microflow systems to provide miniaturized continuous flow displacement assays as a universal technique for mapping functional sites in proteins and other biopolymers.

A. Mapping Functional Sites-Located Functional Motifs.

Consensus sequences have been defined for many post-translational modifications, functional domains, and functional motifs. However, the existence of a consensus sequence does not assure that a protein is modified and functional sites must be confirmed or refuted experimentally. Motifs appear in the primary linear structure of the protein. For example, the sequence RGD is a motif that binds integrins. However, not all RGD sequences in proteins bind integrins, and whether or not they bind must be determined experimentally. In the figure, a peptide with a functional binding motif is labeled and bound to its immobilized receptor. A protein or polypeptide suspected of having the functional motif is injected into the flow chamber. If the functional motif is present, it can displace the bound molecules which can be detected downstream. In like manner, post-translational modifications, or any other site on the surface of a protein or other biopolymer that reversibly binds a ligand can be rapidly identified using these miniaturized continuous flow chips.

### Example 31

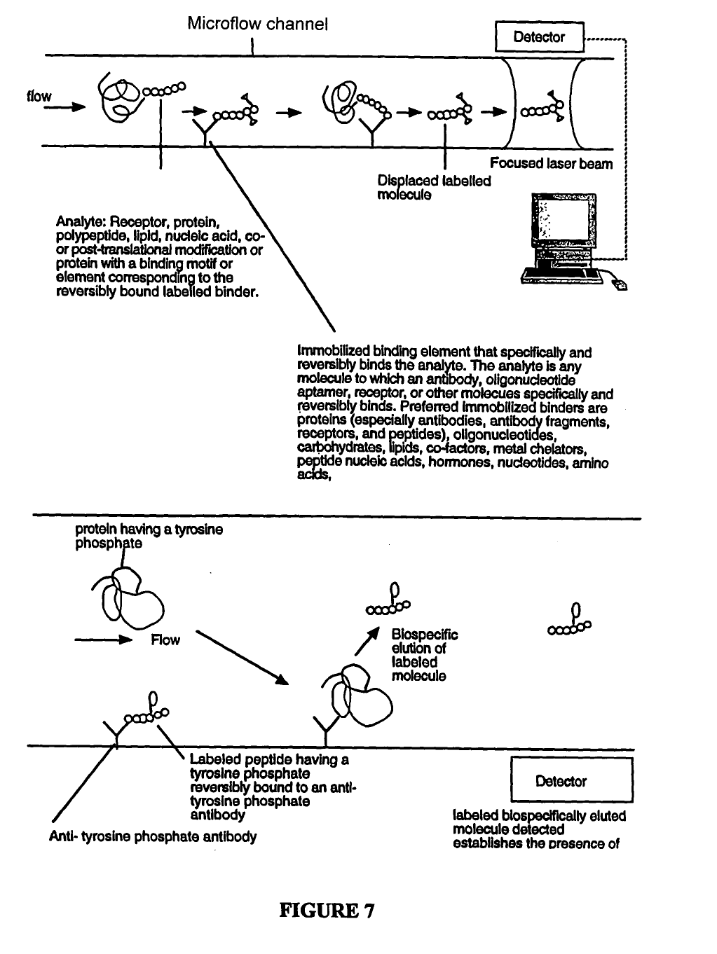

FIG. 7 illustrates the use of the inventive methods and microflow systems to study protein-protein interactions using a competitive displacement desorption to detect a modified protein residue by use of a modification-specific antibody. The analyte may be a receptor, protein, polypeptide, lipid, nucleic acid, co- or post-translational modification or protein with a binding motif or element corresponding to the reversibly bound labeled binder. The immobilized binding element that specifically and reversibly binds the analyte may be any of a number of binders. The analyte is any molecule to which an antibody, oligonucleotide aptamer, receptor, or other molecules specifically and reversibly binds. Preferred immobilized binders are proteins (especially antibodies, antibody fragments, receptors, and peptides), oligonucleotides, carbohydrates, lipids, co-factors, metal chelators, peptide nucleic acids, hormones, nucleotides, amino acids.

### Example 32

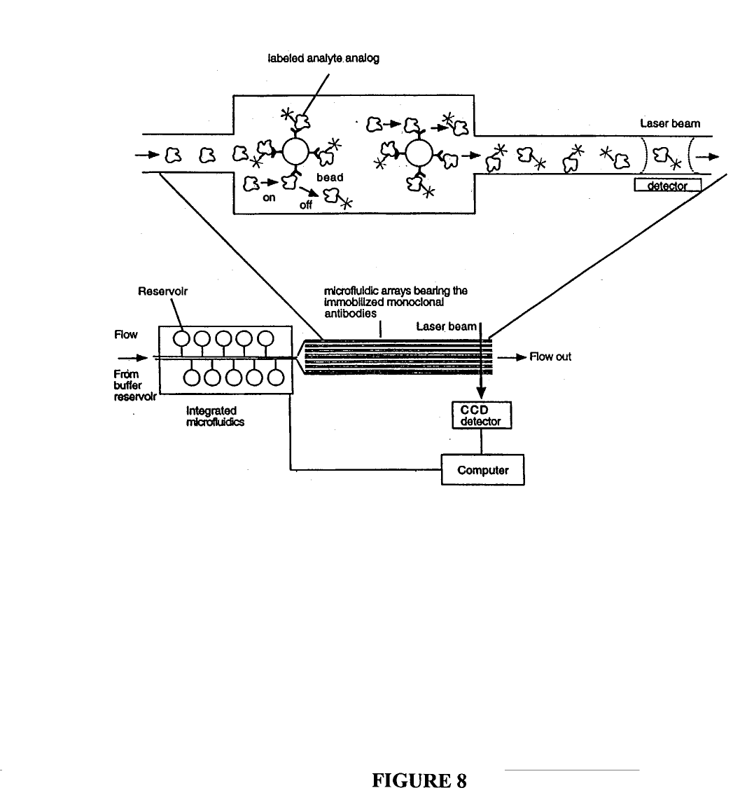

FIG. 8 illustrates an automated high throughput screening microsystem using continuous biospecific desorption for the isolation of antibodies having desired affinity properties. Antibodies are used in various ways throughout biology. For example, antibodies are used in liquid chromatography for the purification of proteins and other substances. Antibodies used for these purposes must have certain desirable affinity characteristics. For example, the antibody must bind strongly enough to retain the antigen being purified but loosely enough so that the antigen can be eluted. Automated high throughput screening systems are needed to determine the affinity characteristics of antibodies on a microscale.

Each antibody (the antibody may be a polyclonal or monoclonal antibody or a fragment there of) is immobilized to a surface (e.g. a nanoparticle, bead, optical fiber or microelectrode) and positioned in a separate microflow channel. The antibodies are saturated with the labeled analyte. The unlabeled analyte is perfused through the microflow channels at different concentrations. This may be achieved in an automated, computer-controlled microsystem by having the analyte transported from reservoirs by microfluidic pumps. Each reservoir can contain the analyte at different concentrations. The labeled analyte can be displaced by the unlabeled analyte and detected. As shown in this embodiment, the labeled analyte analog is eluted and flows past a detector and is continuously detected. Alternatively, the antibodies may be immobilized onto transducers (e.g. optical fibers or microelectrodes) and the biospecific desorption may be detected by a proportionate decrease in signal marking the desorption. From the concentration of the unlabeled analyte causing the elution and the time required the dissociation rate constant and other binding parameters may be computed. Antibodies and other binders can be studied under multiple elution conditions automatically. This can be achieved by having different buffers and substances causing elutions to be transported from reservoirs automatically and perfused through the antibody containing channels. Importantly, displacement efficiencies can be automatically established by perfusing known concentrations of the antigen through the system using computer controlled and integrated microfluidic systems. This microsystem can be used to select monoclonal antibodies and other binders (e.g. peptides or oligonucleotide aptamers) suitable for reversible binding in continuous elution microsystems invented herein.

### Example 33

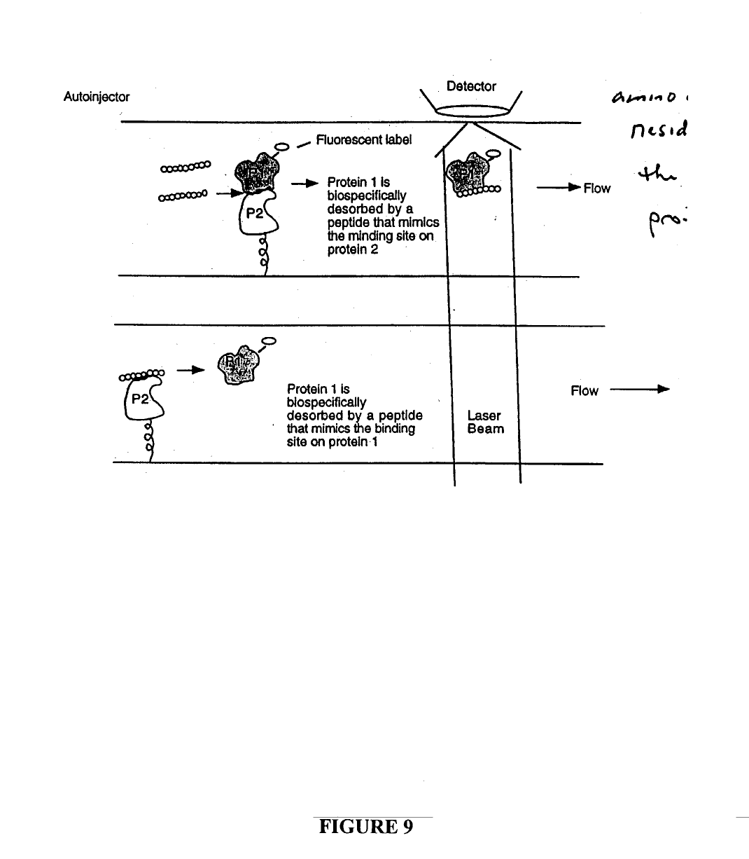

FIG. 9 illustrates a further use of the inventive methods and microflow systems to study protein-protein interactions. Overlapping synthetic peptides can be made corresponding to the amino acid sequences of the interacting proteins. One of the proteins can be immobilized and the other can be allowed to bind. A series of synthetic peptides can be injected into the flow chamber by an autoinjector. When peptides corresponding to the binding sites of the bound proteins mediating their interaction are injected, the bound protein can be eluted and detected by the detector. The detector and autoinjectors can be integrated. The computer-controlled system can have a record of which peptide was injected causing the elution, and hence an automated system for mapping binding sites on protein surfaces can have been created. This method amy be used to screen for drugs (e.g. peptides produced by combinatorial chemistry) that block protein-protein, protein-cell, cell-cell, or cell-virus interactions. In this scheme it is possible to study the binding of multiple protein-protein, cell-cell, protein-cell, or other ligand-receptor interactions in the same microflow channel. Each labeled component of the ligand-receptor pair can be distinguishable. For example a single optical detector can simultaneously detect and distinguish between different fluorescent labels. In a like manner, different microparticles or beads can be distinguished using encoding schemes or different sizes colors and the like. Flow cytometers that can distinguish between a multitude of such labels are known in the art. High throughput is achieved by the following advantages: microflow systems are rapid because microflow transport provides convective mass transport and the small dimensions eliminate diffusional limitations, a multitude of microchannels may be detected simultaneously, and multiple binding interactions may be studied in the same microchannel using different labels to identify unique binding elements.

### Example 34

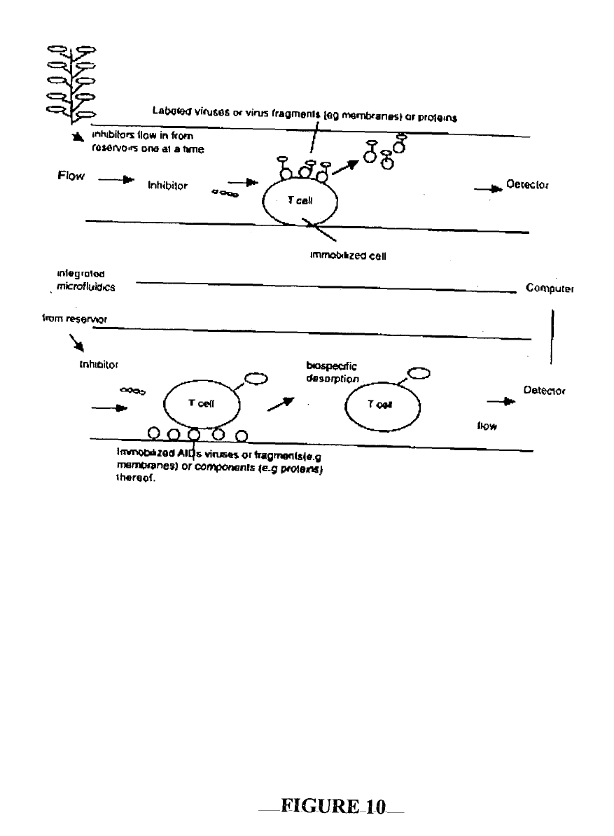

FIG. 10 illustrates the use of the inventive methods and microflow systems to study protein-protein interactions related to AIDS. A battery of drugs aimed at different stages HIV's life cycle can enable switching of treatments when resistant viruses emerge or if patients are unable to tolerate established therapies. Intense efforts are now underway to produce drugs that target chemokine receptors used by HIV to gain entry into the cell. HIV needs two receptors on the host cell surface for efficient attachment and infection. The virus first attaches to CD4 but requires a coreceptor to penetrate the cell membrane. The first co receptor, identified in 1996, is a member of the chemokine receptors (the G-protein coupled 7TM superfamily). Indeed, many small, orally bioavilable molecules that block various 7TM receptors are used to treat numerous diseases including ulcers, allergies, migraines, and schizophrenia are known.

These molecules are the cornerstone of the pharmaceutical industry's contribution to fight against a multitude of diseases. Using these microsystems, it can be possible to screen for small molecule inhibitors of receptors in a highly paralleled and automated manner on a microscale thereby enabling the development of drugs for fighting AIDS and other diseases. Integrated and computer-controlled Microsystems for rapid high throughput screening of inhibitors that block AIDS virus binding to cells. AIDs viruses or cells that bind the virus (or fragments or components thereof) are immobilized in microflow channels. For example, HIV-1 envelope glycoprotein, gp120 binds to CD4 and is necessary for virus entry. Hence, gp120 may be immobilized in the microflow channels or labeled gp120 may be reversibly bound to CD4 that is immobilized in the flow channels. Potential inhibitors that block this interaction are automatically perfused through the channels being transported from reservoirs by computer-controlled microfluidics. Inhibitors causing a desorption can thereby be identified as described. The labeled virus, cell or component is adsorbed to the immobilized cognate binder. Using integrated microfluidics, potential inhibitors are transported from reservoir arrays through the microflow channels bearing the immobilized binding elements. Inhibitors that cause a biospecific desorption can be identified. This can be accomplished by the detection of the eluted labeled molecule and the reservoir containing the inhibitor that caused the elution. The computer can control the fluidic inputs into the biorecogniton channel and by integrating the detector and microfluidics on the chip the identity of the reservoir and hence the inhibitor can be computed. In a like manner, different concentrations of potential inhibitors can be transported from different reservoirs. Hence, the concentration of inhibitors causing the elution can be computed automatically. The computer can be programmed for binding data analysis. Using this approach, it can be possible to screen for inhibitors for mutant viruses.

### Example 35

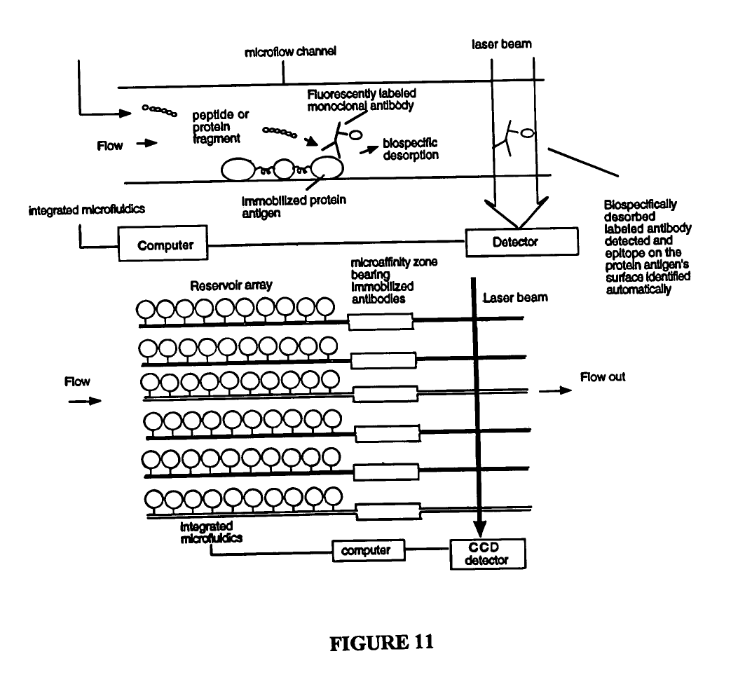

FIG. 11 illustrates the use of the inventive methods and microflow systems for epitope mapping using microflow biospecific desorption.

(1). The antigen protein is immobilized in a microflow channel. Alternatively, the antibody may be immobilized in the channel and the labeled protein antigen reversibly bound.

(2). A unique labeled (e.g. fluorescently labeled) monoclonal antibody can be bound to the protein antigen immobilized in the microflow channel.

(3). Peptides having sequences corresponding to the amino acid sequences on the immobilized protein can be flowed through the microflow channel one at a time. Each unique peptide of known sequence can be transported from a different reservoir using integrated microfluidic transport. Overlapping peptides corresponding to the entire amino acid sequence of the immobilized protein may be used.

(4). Because the sequence of each peptide causing the desorption of the labeled antibody is known, and because each unique peptide is transported from a different reservoir in a computer controlled and integrated manor, the epitope can be identified from the integrated detection of the labeled antibody.

(5). For protein antigens, epitope may involve a single length of the polypeptide chain or may be composed of several widely separated, discrete amino acid sequences that come together in the folded native protein (discontinuous epitopes). The protein antigens may be fragmented (for example, using limited proteolysis with trypsin) and the fragments separated and identified. Each fragment corresponding to a separate domain may then be perfused through the microflow channel bearing the immobilized protein. The protein fragment causing the biospecific desorption of the labeled antibody can be the fragment bearing the epitope for the antibody.

### Example 36

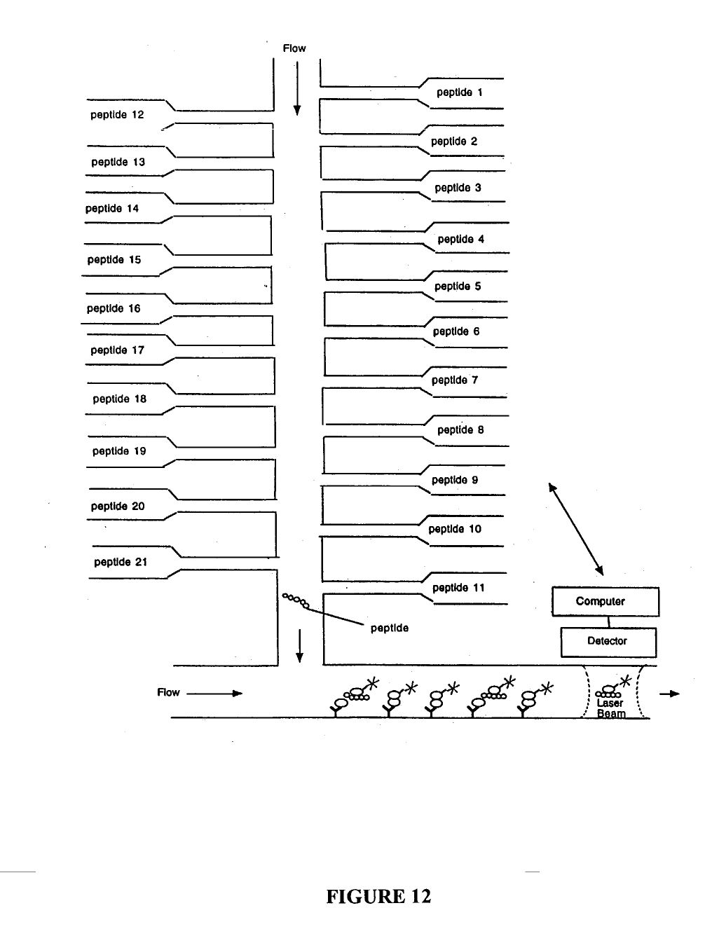

FIG. 12 illustrates the use of the inventive methods and microflow systems for high throughput screening of chemicals such as drugs or, as exemplified, peptides. A series of synthetic peptides corresponding to the amino acid sequences on a protein's surface are used to map the binding sites responsible for protein-protein interactions. Each peptide may be placed in a separate reservoir in fluid connection to the main microflow channel bearing the immobilized protein-protein interacting pair. The different peptides are perfused through the main microflow channel, one-at-a-time by a computer-controlled microsystem. Microfluidic devices (valves and microfluidic pumps) permit the controlled addition of the different peptides in an automated manner. The binding sites can be identified by the peptides causing the biospecific desorption. In an analogous manner protein domains, motifs, of active sites may be analyzed.

### Example 37

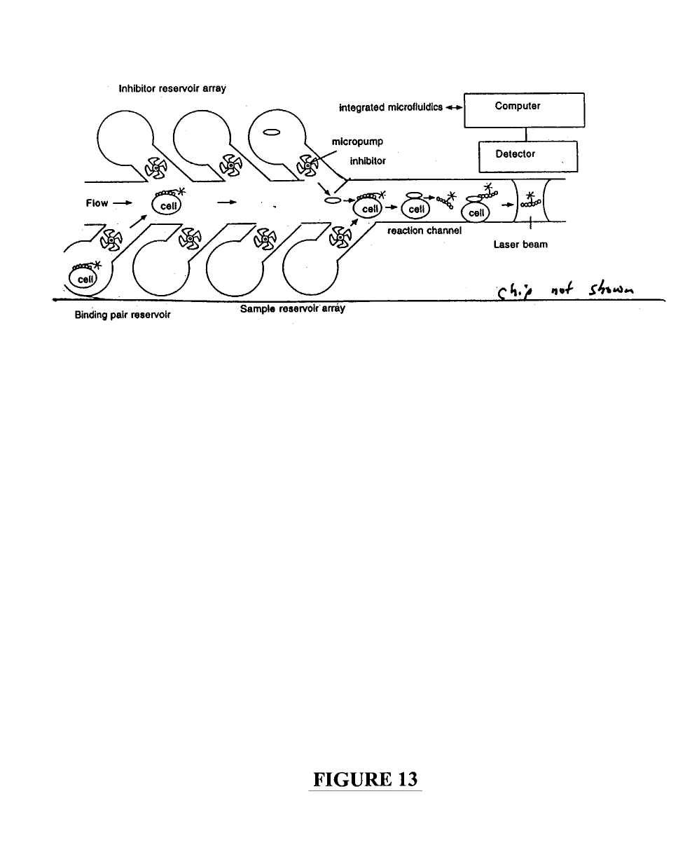

FIG. 13 schematically illustrates a microflow system using a homogeneous fluorescent binding assay to detect inhibitors of cell surface receptor-ligand interactions. A suspension of cells binding to the fluorescently labeled ligand (for example a peptide) may be perfused through the reaction channel that can be integrated with a light source and a fluorescent detector. Potential inhibitors can be perfused through the reaction channel, one at a time, each from a separate reservoir and being transported through the reaction channel using computer controlled microfluidic pumping. Tens to thousands of such reservoirs are in fluid connection to the reaction channel and potential inhibitors of the biospecific interaction are automatically perfused. The desorption event is monitored continuously by any number of fluorescence techniques that are well known in the arts. For example fluorescence polarization, fluorescence correlation spectroscopy, or fluorescence energy transfer. (See Tetin, S Y and Hazlett, T L (2000) Methods 20:341-61 for review on fluorescence polarization, fluorescence energy transfer and fluorescence correlation techniques for monitoring ligand-receptor interactions) may be used as well as others. The identity of the inhibitor can be established automatically from the location of the well causing the desorption. And the strength of the inhibitor (i.e., the Kᵢ) may be estimated automatically by the degree of biospecific desorption caused by a known concentration of each inhibitor. Different known concentrations of each inhibitor may be transported from each well to compute the affinity constants. A different binding pair may be transported from a separate reservoir and mixed with a series of inhibitors each being transported from a separate reservoir, one at a time. The integrated detector can continuously monitor the extent of inhibition (the amount of labeled ligand desorbed from the complex) for each inhibitor and the data can be recorded. This cycle of computer controlled mixing of inhibitors and binding pairs with automated data acquisition and analysis can permit automated high through put screening of inhibitors on a microscale. Tens to thousands of samples may be studied using a single microsystem.

### Example 38

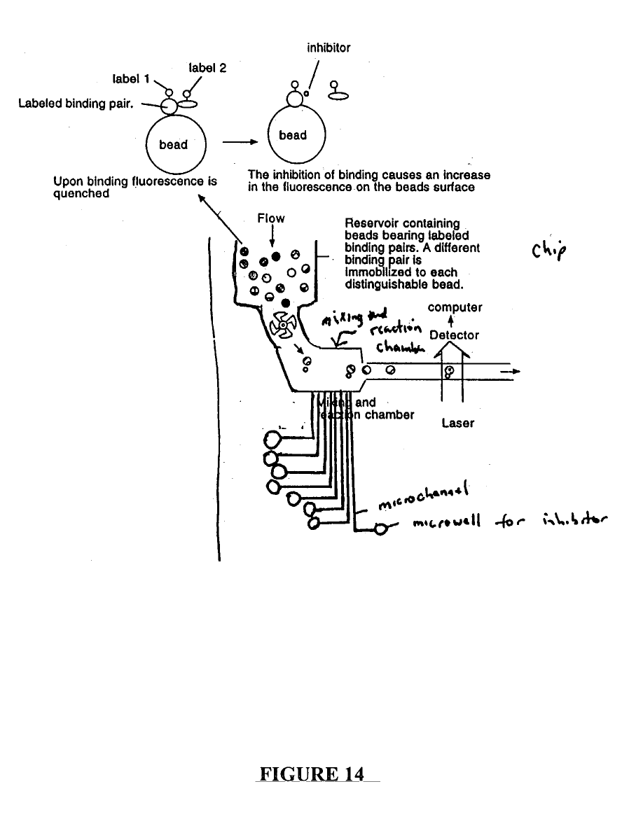

FIG. 14 illustrates a microflow array for the automated analysis of the inhibition of biospecific interactions using two labels and fluorescence detection. A different binding pair can be immobilized to a bead, microsphere, vesicle or other particle that may be distinguished by the detector. For example different color or different size beads may be distinguished or different encoding schemes may be used. (For detector schemes that distinguish between different microspheres, see, for example, U.S. Pat. No. 5,736,330; Wilson et al., Journal of Immunological Methods, 107; 225-230 (1988) 107; 225-230; Karri L M, et al. (1998) Anal Chem 70:1242-1248.) Inhibitors are transported from reservoirs, one at a time, and mixed with microspheres bearing the labeled binding pairs during continuous flow. As beads flow past the detector, the extent of binding for each pair on the distinguishable micro spheres can be computed. This may be accomplished by using double labeling schemes. Each member of the binding pair may be labeled with a different fluorescent dye such that the fluorescence is heavily quenched upon binding. An increase in fluorescence would result on each bead that is proportional to the inhibition of the specific binding by an inhibitor. Other double labeling schemes would be suitable for this embodiment. For example, one label may be attached to the microsphere and the other conjugated to a binding member. Detection schemes may include fluorescence energy transfer, fluorescence quenching techniques, fluorescence detection, or determining the ratio of two labels on the beads surface. For example, the first label may be Texas red and the second label may be fluorescein and the ratio of fluorescein to Texas Red on the microsphere's surface may be determined by dual-channel laser confocal microscope as a detection system (see, for example, U.S. Pat. No. 5,171,695 (1992) Issued to Ekins).

### Example 39

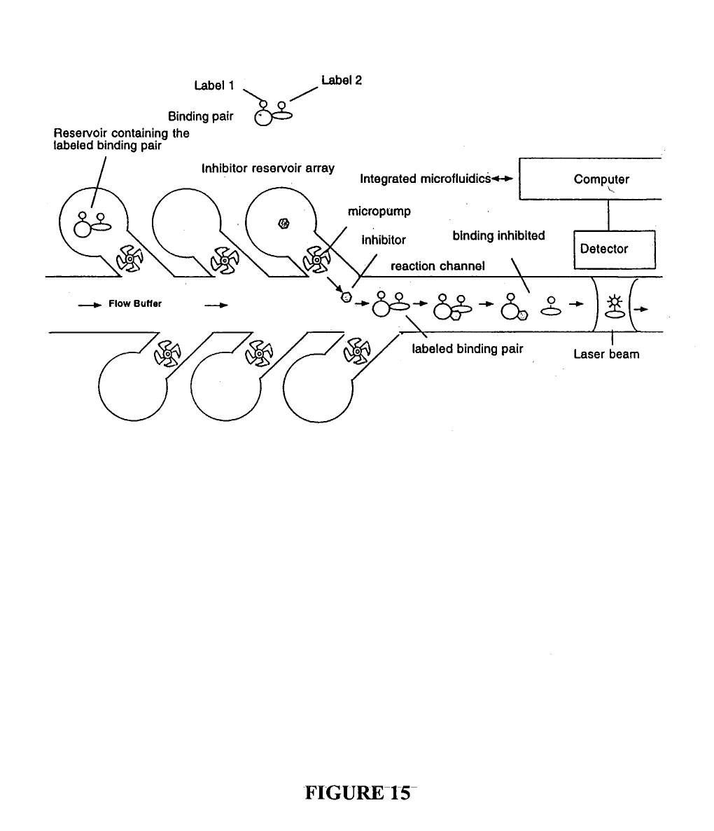

FIG. 15 schematically illustrates an automated microsystem suitable for screening for inhibitors, activators, or co-factors of biospecific interactions using an energy transfer assay. The ligand and receptor are labeled with an energy donor and acceptor. The binding pair or complex may be composed of any specific biomolecular interactions. Relevant interactions include protein-protein, protein-phage, protein-cell, protein-DNA, protein-RNA, cell-cell, cell-virus, cell-bacterium, protein-drug, protein-carbohydrate, protein-lipid interactions. The binders are labeled with dyes such that their fluorescence is heavily quenched when they are bound. The release of the bound molecules by the inhibitor generates an increase in fluorescence that is proportional to the amount of inhibition. The decrease in fluorescence is related to changes in the amount of complex that is bound at any time. In a like manner this set up may be used for screening for activators or co-factors for binding partners or binding complexes. An activator would lead to a higher proportion of bound binding partners at any time (i.e., a lower dissociation constant). Hence the same microsystem may be used for screening for activators, co-factors, or inhibitors of biospecific interactions. A large selection of dyes are commercially available for use in fluorescence energy transfer assays which are well known in the arts. (Haugland, R. P. (1992) Handbook of Fluorescent Probes and Research Chemicals, 5th ed., Molecular Probes, Eugene, Oreg.; Jones, L J et al (1997) Analytical Biochemistry 251:144-152; Matayoshi, E D (1990) Science 247:954-957; Tetin, S Y and Hazlett, T L (2000) Methods 20: 341-61). As shown two flow streams are joined into a reaction channel. One stream carries potential inhibitors from separate reservoirs. The other stream carries the labeled binding pair which can be transported from a separate reservoir via microfluidic pumping. The computer linked detector records the response of each inhibitor on the binding pair automatically. The same microsystem may be used to automatically perfuse different concentrations of the inhibitors through the reaction channel in order to compute the affinity constants of inhibitors. Multiple binding pairs may be screened for inhibitors, activators or co-factors. Each different labeled binding pair can be transported from a different reservoir and mixed with a potential inhibitor, activator, co-factor, one at a time, each being transported form a separate reservoir. The computer records the binding data for each binding pair and reagent. Tens to thousands of binding pairs may be analyzed in the presence of tens to thousands of potential inhibitors or activators on a microscale in a single automated microsystem.

### Example 40

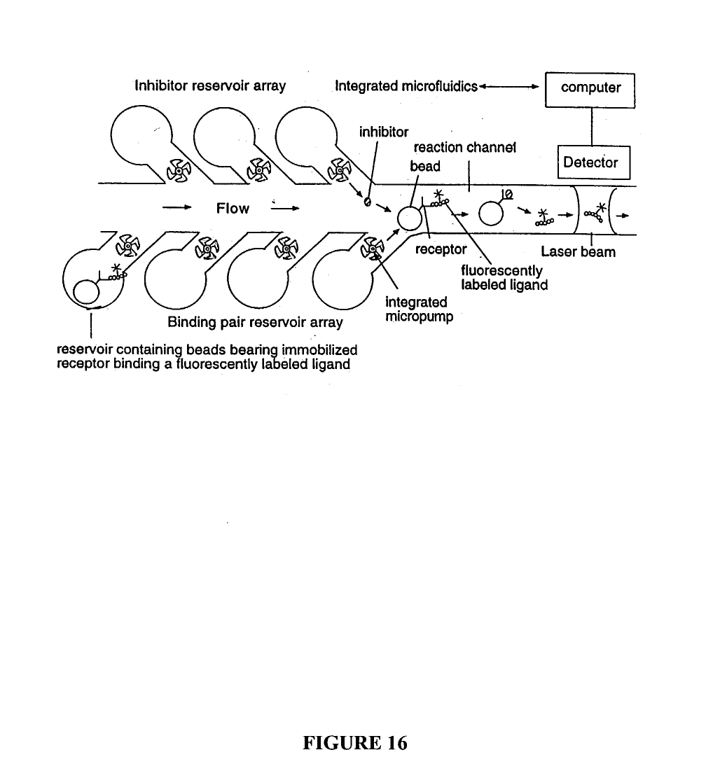

FIG. 16 is schematic drawing of a micro flow system employing integrated fluorescence polarization to detect the inhibition of ligand-receptor interactions. One binder can be immobilized on a bead, phage, vesicle, cell, nanoparticle or the like and bound to a labeled ligand. Inhibitors are perfused through the reaction channel one at a time from a separate reservoir. The flow stream containing the bead immobilized binding pair can join the flow stream carrying the inhibitor in the reaction channel. As inhibitors block the biospecific binding, the increase in the amount of fluorescently labeled ligand that is unbound can be continuously monitored by a fluorescence technique such as fluorescence polarization, fluorescence energy transfer, or fluorescence correlation spectroscopy. It is possible to test multiple binding pairs in the same microsystem by having separate reservoirs for different binding pairs. Each binding pair can be transported from its unique reservoir and can be mixed with a different inhibitor that is transported from a separate reservoir. The inhibition data (extent of inhibition) can be recorded for each inhibitor. Then the next binding pair can automatically be transported from a separate reservoir and combined with a series of inhibitors each being transported from a separate reservoir, one at a time. The extent of inhibition for each inhibitor can be recorded. This cycle can automatically continue for as many as tens to thousands of inhibitors and binding pairs all being automatically screened with automated data acquisition and analysis. It is also possible to screen multiple binding pairs simultaneously by having each binding pair immobilized on a distinguishable bead. The beads may be distinguishable by different encoding schemes or by being different colors or sizes as disclosed in U.S. Pat. No. 5,736,330.

### Example 41

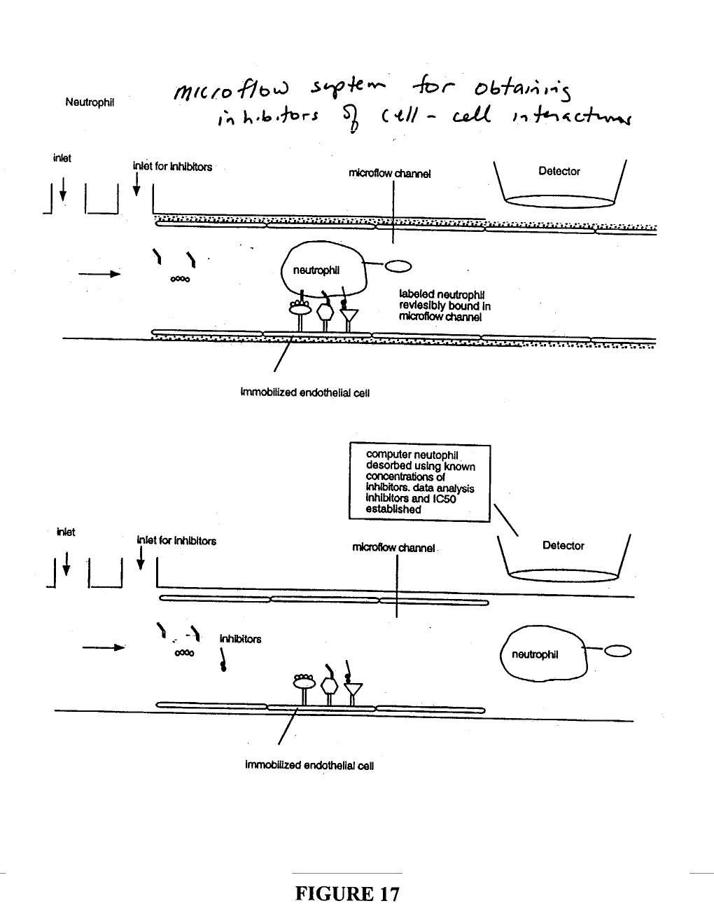

FIG. 17 is a schematic representation of a microflow system for studying cell to cell interactions as exemplified by neutrophil and monocyte adhesion to endothelial cell in a microflow channel. Once endothelial cells are activated by inflammatory agents (as added by inlets) selectins are transported to the cell surface and bind to leukocytes resulting in the slow down leukocyte or rolling effect. Once leukocytes are close to the endothelial cells because of the chemoattractants such as MIP, originally bound to the cell surface heparin sulfate are transferred to a receptor. Active integrins now bind to the ICAM-1 in endothelial cells, establishing tight binding to endothelials. The last step then leads to penetration of endothelial cells, and vascular extravasation.

### Example 42

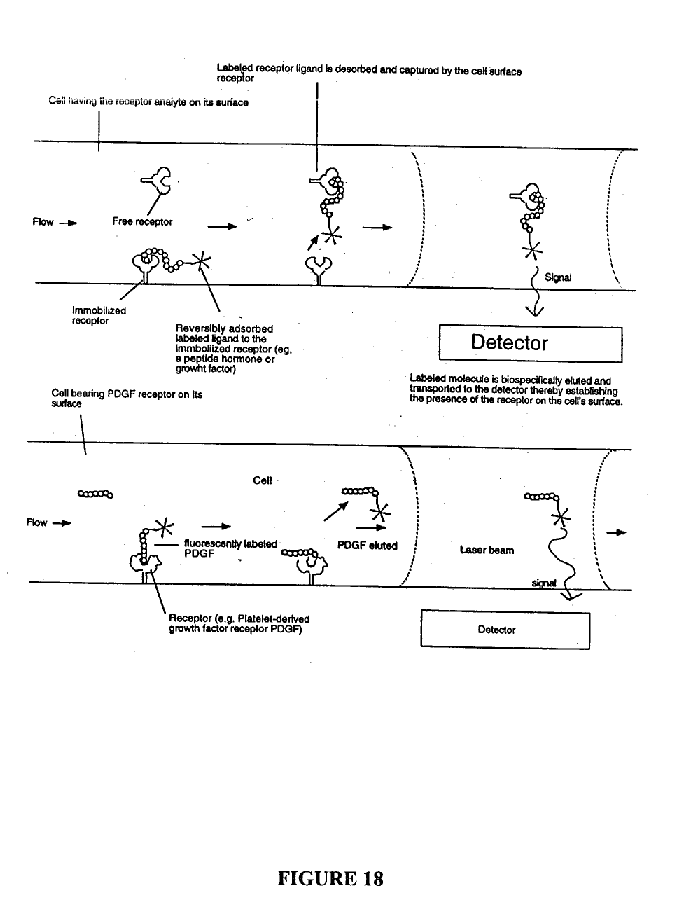

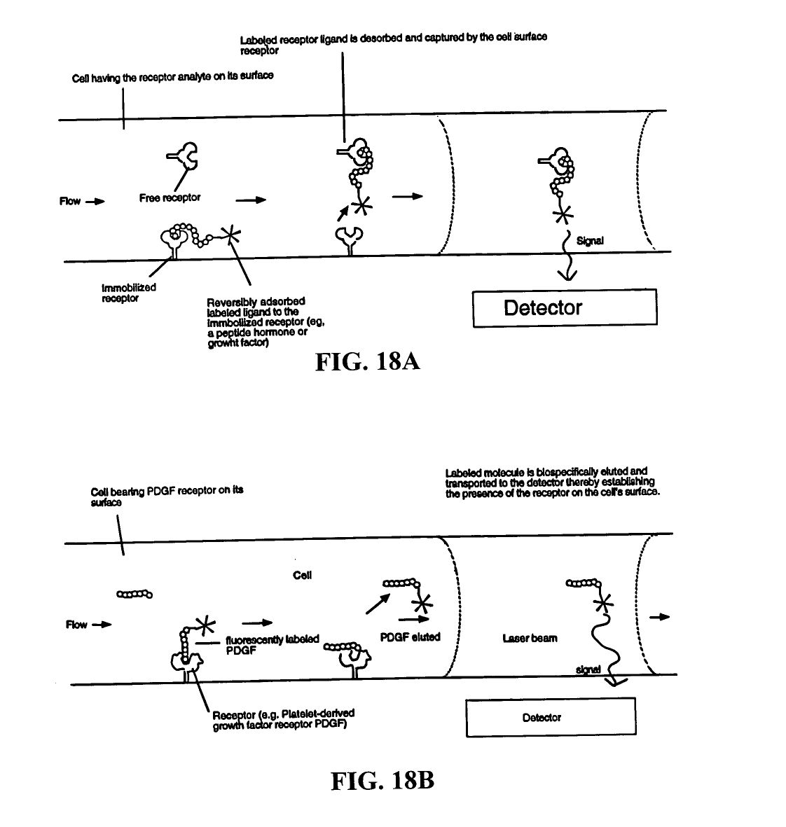

FIG. 18A is a schematic depiction of a rapid automated microfluidic chip for determining the presence and/or amount of a receptor to a drug or hormone in a sample using biospecific desorption during flow. The receptor can be flowed through a microchannel having the receptor immobilized within the microflow channel and reversibly adsorbed to a labeled ligand which specifically binds the receptor. The labeled ligand (e.g. a hormone or drug) can be competitively displaced from the immobilized receptor by the free receptor. The labeled ligand then flows past the integrated detector and can be detected.

FIG. 18B. depicts a rapid automated microfluidic chip for determining the presence and/or amount of a hormone in a sample. The hormone can be flowed through a microflow channel that has a receptor to the hormone immobilized within. A labeled (e.g. fluorescently labeled) hormone that specifically and reversibly binds the immobilized receptor can be bound to the immobilized receptor. As the peptide in the sample is flowed through the microflow channel, it competitively displaces its labeled analog which can be detected by the detector.

### Example 43

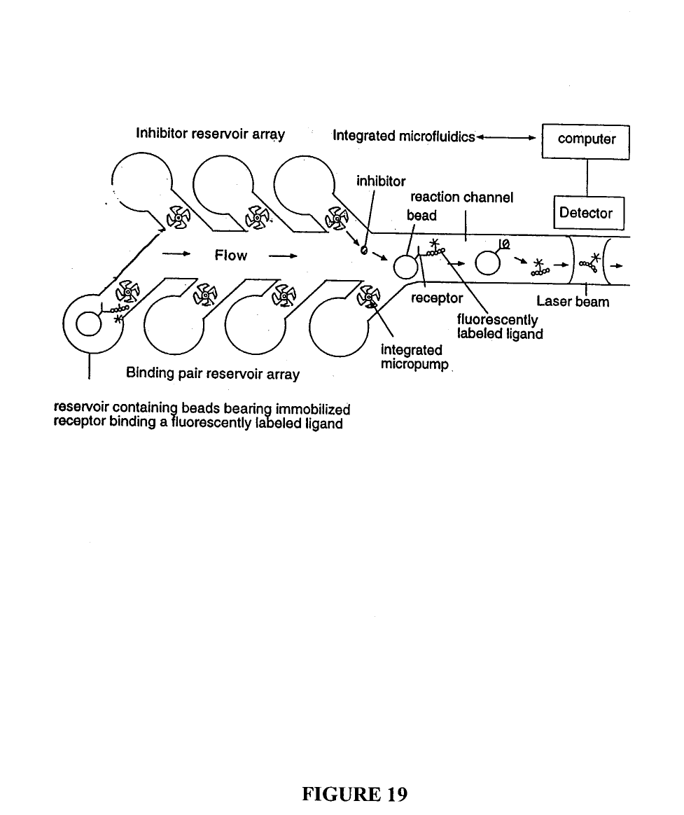

FIG. 19 is a schematic drawing of a microflow system employing integrated fluorescence polarization to detect the inhibition of ligand-receptor interactions. One binder is immobilized on a bead, phage, vesicle, cell, nanoparticle or the like and bound to a labeled ligand. Inhibitors are perfused through the reaction channel one at a time from a separate reservoir. The flow stream containing the bead immobilized binding pair can join the flow stream carrying the inhibitor in the reaction channel. As inhibitors block the biospecific binding, the increase in the amount of fluorescently labeled ligand that is unbound can be continuously monitored by a fluorescence technique such as fluorescence polarization, fluorescence energy transfer, or fluorescence correlation spectroscopy. It is possible to test multiple binding pairs in the same microsystem by having separate reservoirs for different binding pairs. Each binding pair can be transported from its unique reservoir and can be mixed with a different inhibitor that can be transported from a separate reservoir. The inhibition data (extent of inhibition) can be recorded for each inhibitor. Then the next binding pair can automatically be transported from a separate reservoir and combined with a series of inhibitors each being transported from a separate reservoir, one at a time. The extent of inhibition for each inhibitor can be recorded. This cycle can automatically continue for as many as tens to thousands of inhibitors and binding pairs all being automatically screened with automated data acquisition and analysis. It is also possible to screen multiple binding pairs simultaneously by having each binding pair immobilized on a distinguishable bead. The beads may be distinguishable by different encoding schemes or by being different colors or sizes as disclosed in U.S. Pat. No. 5,736,330.

### Example 44

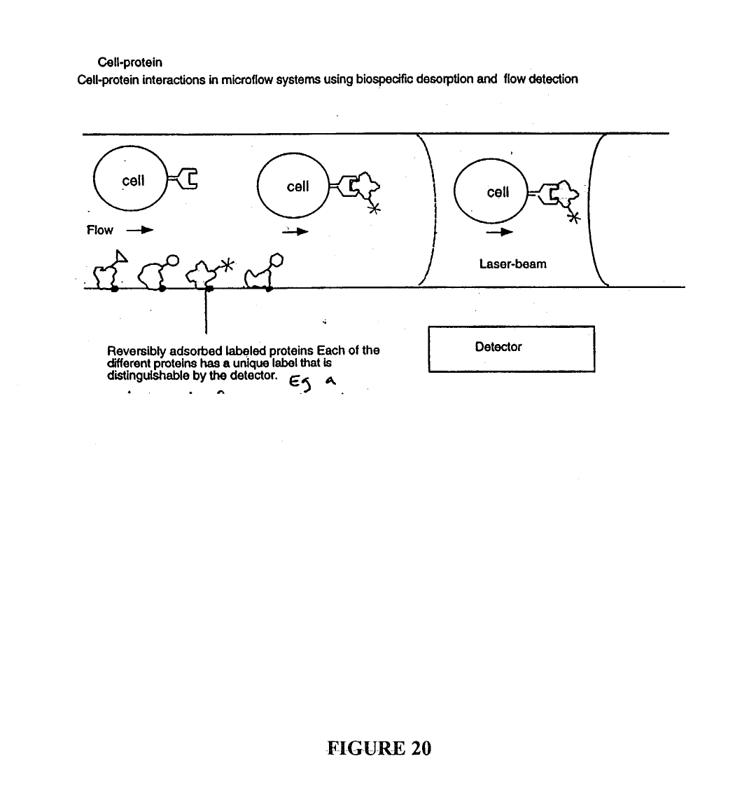

FIG. 20 illustrates the use of the inventive methods and microflow systems to study cell-protein interactions in microflow systems using biospecific desorption and flow detection. Many proteins having important biological and biomedical functions bind to cell surface receptors. Such proteins are especially important in cancer biology, cell migration, blood coagulation and wound healing. Receptor mediated generation of proteases on cellular surfaces is critically involved in regulation of hemostatic, inflammatory, fibrinolytic pathways. These receptors are differentially expressed and the expression changes during disease states. This schematic drawing depicts the determination of a receptor for a protein on a cell surface using the microflow biospecific desorption technique invented herein. The labeled protein can be reversibly adsorbed to its receptor in the microflow channel. The cell bearing the receptor that binds the immobilized protein desorbs the protein and carries it past the detector for detection. The labeled protein can be reversibly adsorbed within the microflow channel. Multiple receptors may be analyzed in the same microflow channel by using different labels. For example if fluorescent labels are used, a single detector can distinguish between a the different labels and thus analyze multiple ligand-receptor interactions in the same microflow channel.

### Example 45

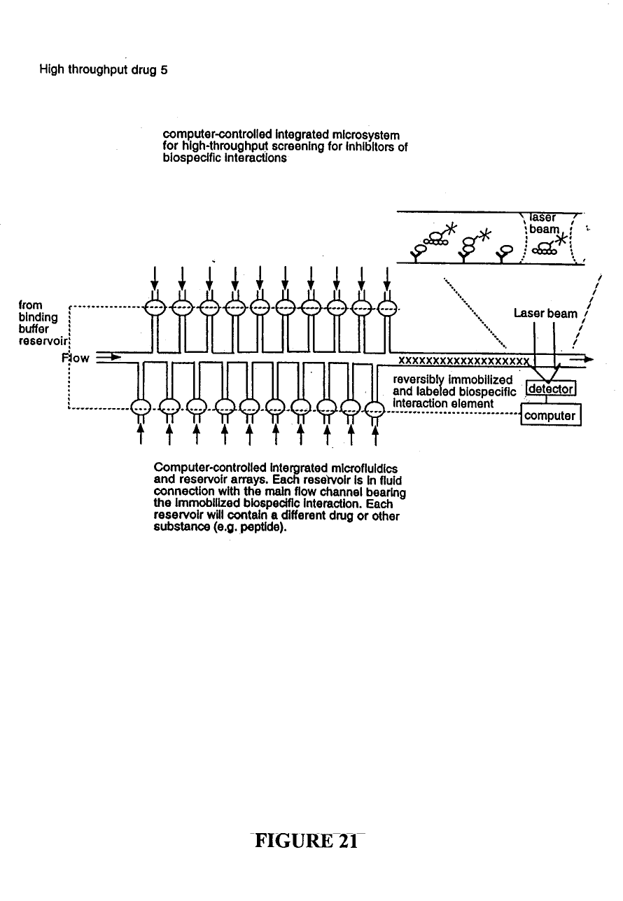

FIG. 21 illustrates the use of the inventive methods and microflow systems for high through put drug screening. This integrated microsystem can be computer-controlled so that a series of drugs or other substances can be perfused through the main microchannel bearing the biospecific interaction. Each different drug or other substance being analyzed as a potential inhibitor for the biospecific interaction can be perfused through the main channel one-at-a-time by the automated microsystem. This can be achieved using integrated microfluidic devices. Once a biospecific desorption occurs, the desorbed labeled element can be detected by the detector and recorded by the computer. In this way the specific reservoir delivering the desorbing substance can be identified. The substance in this reservoir can thereby be identified as the inhibitor of the biospecific interaction. This system can be used for mapping binding sites on the surfaces of cells, proteins, or other biopolymers. For example, for identifying sites on a protein's surface responsible for a biospecific interaction a series of synthetic peptides corresponding to the protein's amino acid sequence can be synthetized. Each reservoir can contain a different peptide. The peptide or combination of peptides causing a biospecific desorption can identify the binding sites on the protein's surface. In a similar manner the microsystem can be used to map specific sequences responsible for protein-nucleic acid interactions, protein-carbohydrate interactions, protein-lipid, interactions, protein-cell interactions and the like.

### Example 46

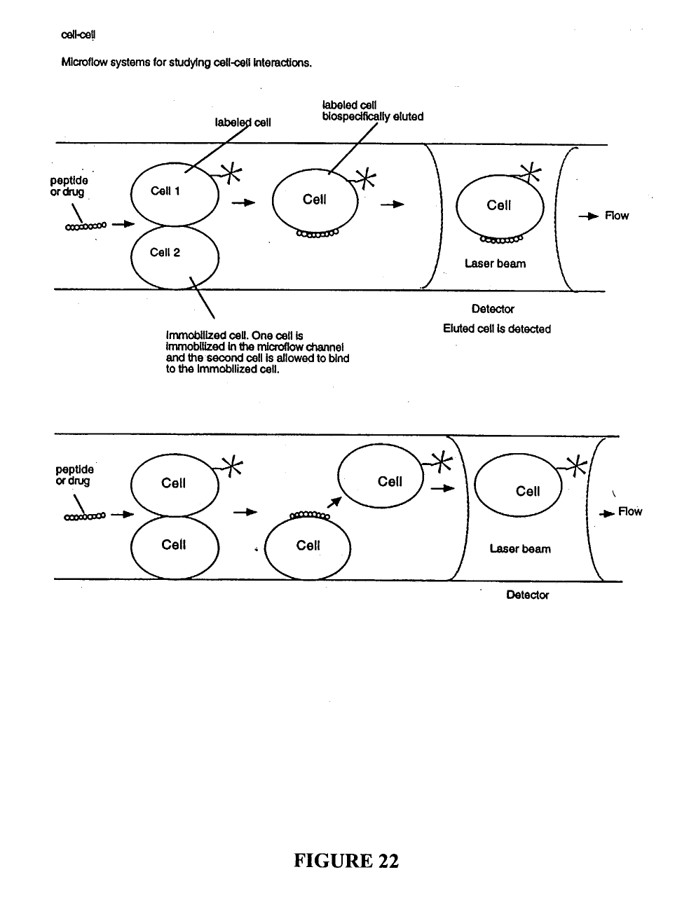

Cell-Cell interactions can be studied in microflow system as illustrated in FIG. 22. Peptides or other substances (e.g. drugs) can be perfused through the microflow system to find substances that inhibit the cell-cell interactions. Peptides or other molecules that mimic the binding sites can biospecifically elute the labeled cell which can be detected down stream. The system can be automated and using autoinjectors a series of peptides can be perfused through the microflow channel. Multiple cell-cell interactions can be analyzed in the same microflow channel by using a different label for each cell type. This method can be especially suitable for high through put screening of therapeutic agents that disrupt specific cell-cell interactions. For example, blood clots form when platelets adhere to one another through protein bridges. The protein fibrinogen binds to proteins on the platelet surfaces called integrins. Synthetic peptides having the sequence RGD, a sequence in the fibrinogen protein responsible for binding to the integrin inhibit blood clot formation by competing with the fibrinogen molecules for the AGO-binding sites on the integrins.

### Example 47

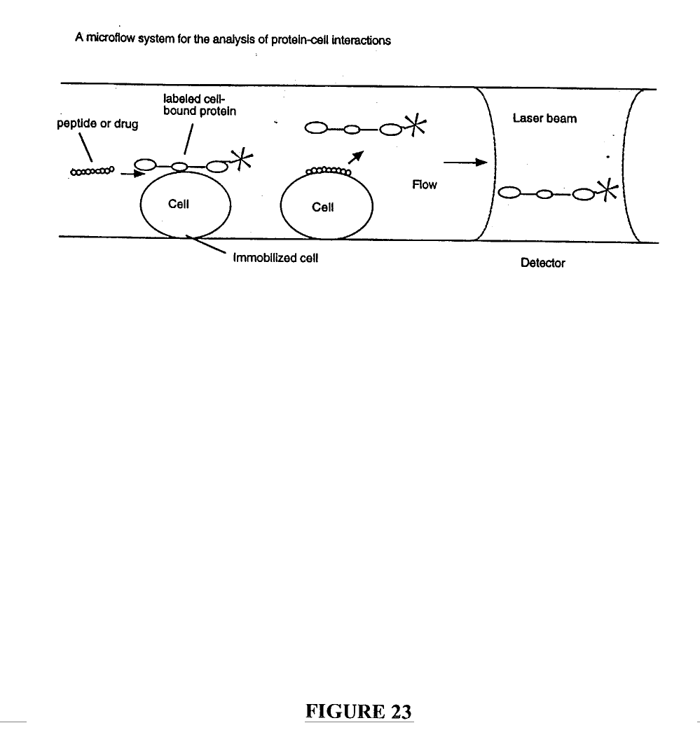

A microflow system for the analysis of protein-cell interactions is shown in FIG. 23. Peptides or other substances (e.g. drugs) can be perfused through the microflow system to find substances that inhibit the cell-protein interactions. Peptides or other molecules that mimic the binding sites can biospecifically elute the labeled protein or cell which can be detected down stream. The system can be automated and using autoinjectors a series of peptides or other substances can be perfused through the microflow channel. Multiple cell-protein interactions can be analyzed in the same microflow channel by using a different label for each specific protein-cell interaction type. This method is especially suitable for high through put screening of therapeutic agents that disrupt specific cell-cell interactions.

### Example 48

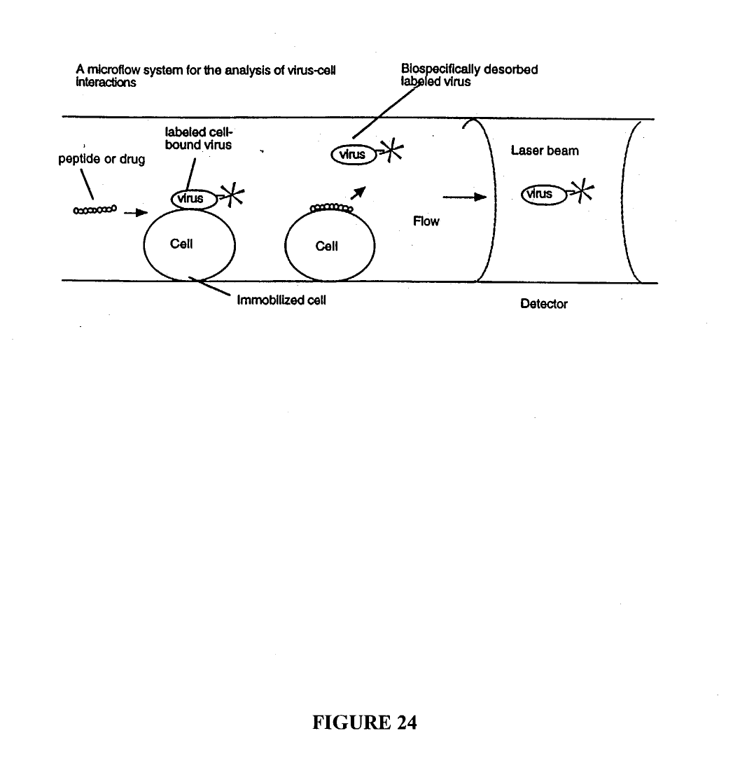

Cell-virus interactions can be studied in microflow system as shown in FIG. 24. Peptides or other substances (e.g. drugs) can be perfused through the microflow system to find substances that inhibit the cell-virus interactions. Peptides or other molecules that mimic the binding sites can biospecifically elute the labeled virus or cell which can be detected down stream. The system can be automated and using autoinjectors a series of peptides or other substances can be perfused through the microflow channel. Multiple cell-virus interactions can be analyzed in the same microflow channel by using a different label for each specific virus-cell interaction type. This method is especially suitable for high through put screening of therapeutic agents that disrupt specific cell-virus interactions.

### Example 49

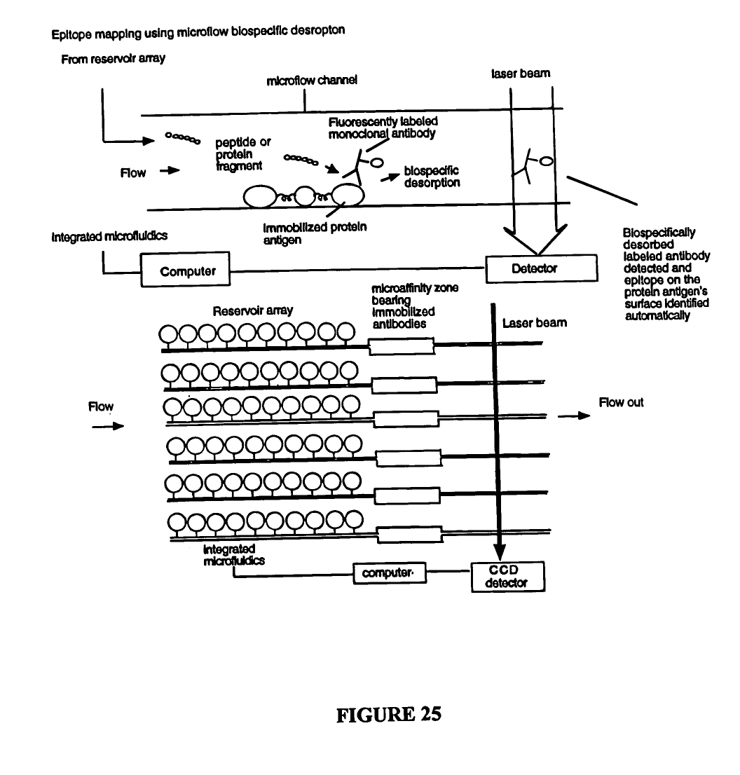

A system of epitope mapping using microflow biospecific desorptions is shown in FIG. 25.

(1). The antigen protein is immobilized in a microflow channel. Alternatively, the antibody may be immobilized in the channel and the labeled protein antigen reversibly bound.

(2). A unique labeled (e.g. fluorescently labeled) monoclonal antibody can be bound to the protein antigen immobilized in the microflow channel.

(3). Peptides having sequences corresponding to the amino acid sequences on the immobilized protein can be perfused through the microflow channel one at a time. Each unique peptide of known sequence can be transported from a different reservoir using integrated microfluidic transport. Overlapping peptides corresponding to the entire amino acid sequence of the immobilized protein may be used.

(4). Because the sequence of each peptide causing the desorption of the labeled antibody is known, and because each unique peptide is transported from a different reservoir in a computer controlled and integrated manor, the epitope can be identified from the integrated detection of the labeled antibody.

(5). For protein antigens, epitope may involve a single length of the polypeptide chain or may be composed of several widely separated, discrete, amino acid sequences that come together in the folded native protein (discontinuous epitopes). The protein antigens may be fragmented (for example, using limited proteolysis with trypsin) and the fragments separated and identified. Each fragment corresponding to a separate domain may then be perfused through the microflow channel bearing the immobilized protein. The protein fragment causing the biospecific desorption of the labeled antibody can be the fragment bearing the epitope for the antibody.

### Example 50

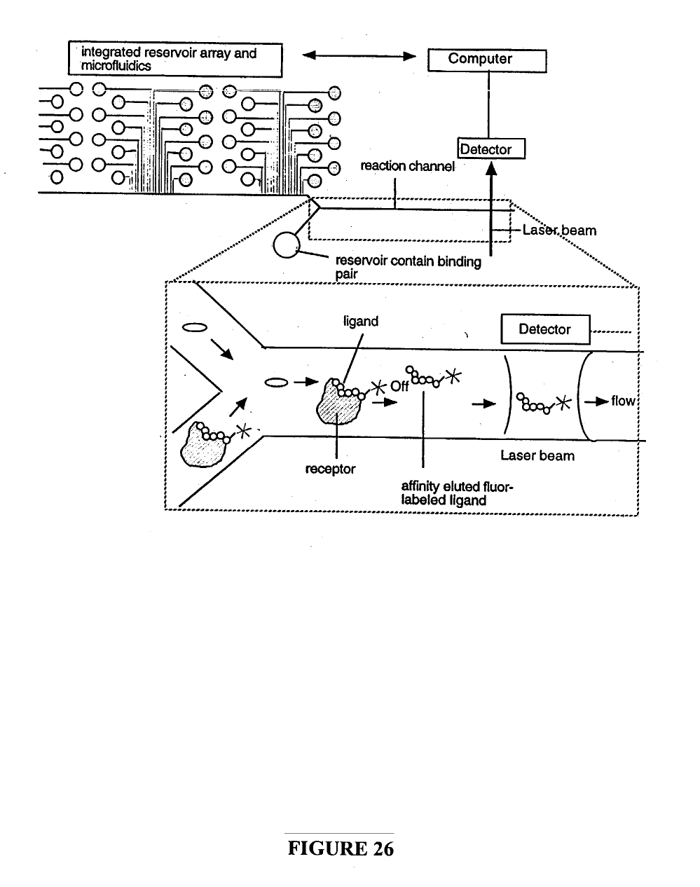

FIG. 26 is a schematic drawing of an integrated microflow system suitable for automated screening of inhibitors of biospecific interactions using integrated fluorescence polarization as a detection assay. The reservoir array is in fluid communication with the reaction channel. Each reservoir in the array contains a unique test sample (potential inhibitor). Inhibitors are perfused through the reaction channel in which the binding pair of interest is continuously flowing. The binding pair is transported from a separate reservoir through the reaction channel, for example, by continuous flow micropumps. One member of the binding pair (the smaller member) can be labeled with a fluor. The labeled ligand may be a ligand for a receptor or a competitive inhibitor for an enzyme. As inhibitors diminish the interaction, the affinity eluted labeled binder can be continuously monitored by the change in fluorescence polarization.

### Example 51

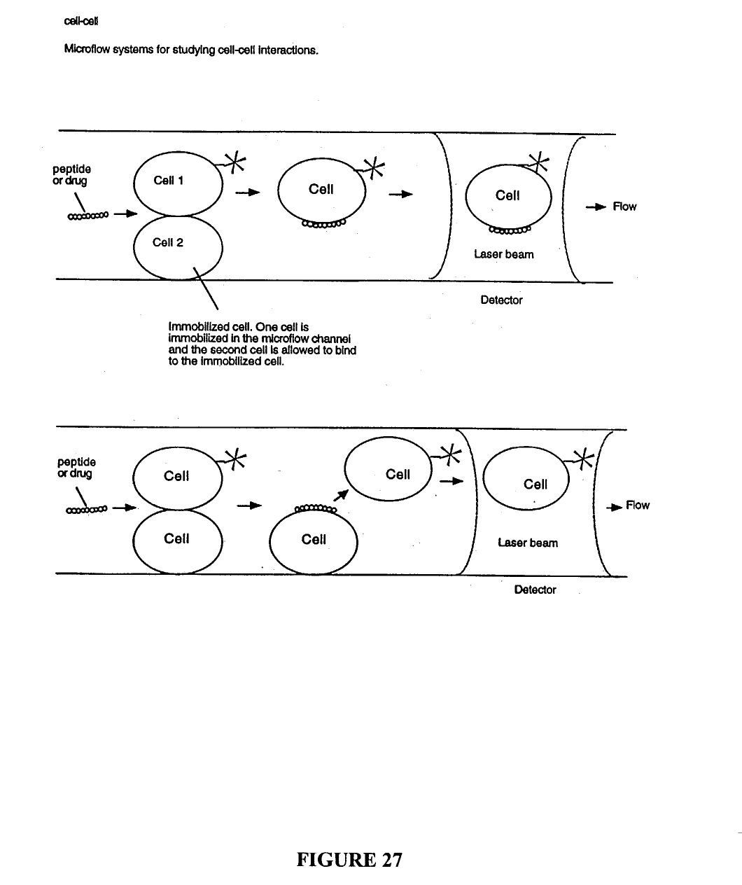

Cell-Cell interactions can be studied in microflow system as shown in FIG. 27. Peptides or other substances (e.g. drugs) can be perfused through the microflow system to find substances that inhibit the cell-cell interactions. Peptides or other molecules that mimic the binding sites can biospecifically elute the labeled cell which can be detected down stream. The system can be automated and using autoinjectors a series of peptides can be perfused through the microflow channel. Multiple cell-cell interactions can be analyzed in the same microflow channel by using a different label for each cell type. This method is especially suitable for high through put screening of therapeutic agents that disrupt specific cell-cell interactions. For example, blood clots form when platelets adhere to one another through protein bridges. The protein fibrinogen binds to proteins on the platelet surfaces called integrins. Synthetic peptides having the sequence RGD, a sequence in the fibrinogen protein responsible for binding to the integrin inhibit blood clot formation by competing with the fibrinogen molecules for the RGD-binding sites on the integrins.

### Example 52

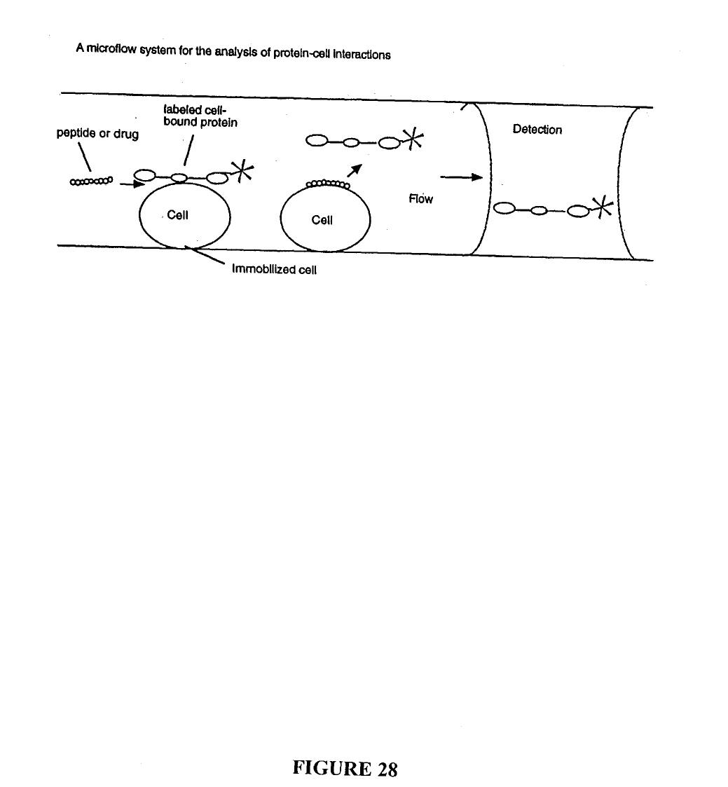

Cell-protein interactions can be studied in microflow system as shown in FIG. 28. Peptides or other substances (e.g. drugs) can be perfused through the microflow system to find substances that inhibit the cell-protein interactions. Peptides or other molecules that mimic the binding sites can biospecifically elute the labeled protein or cell which can be detected down stream. The system can be automated and using autoinjectors a series of peptides or other substances can be perfused through the microflow channel. Multiple cell-protein interactions can be analyzed in the same microflow channel by using a different label for each specific protein-cell interaction type. This method is especially suitable for high through put screening of therapeutic agents that disrupt specific cell-cell interactions.

### Example 53

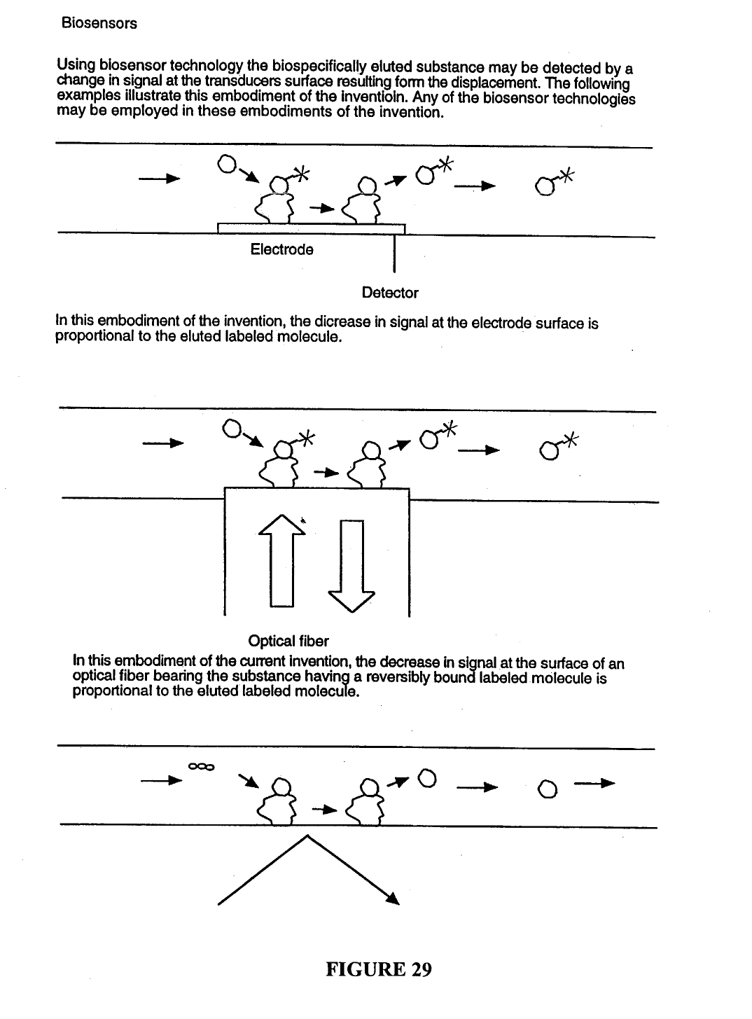

FIG. 29 illustrates the use of biosensor technology. The biospecifically eluted-substance may be detected by a change in signal at the transducers surface resulting form the displacement. The following examples illustrate this embodiment of the invention. Any of the biosensor technologies may be employed in these embodiments of the invention.

In the embodiment at the top of the figure, the decrease in signal at the electrode surface is proportional to the eluted labeled molecule.

In the embodiment at the middle, the decrease in signal at the surface of an optical fiber bearing the substance having a reversibly bound labeled molecule is proportional to the eluted labeled molecule.

In the embodiment at the bottom, the signal is according to the plasmon surface detector.

### Example 54

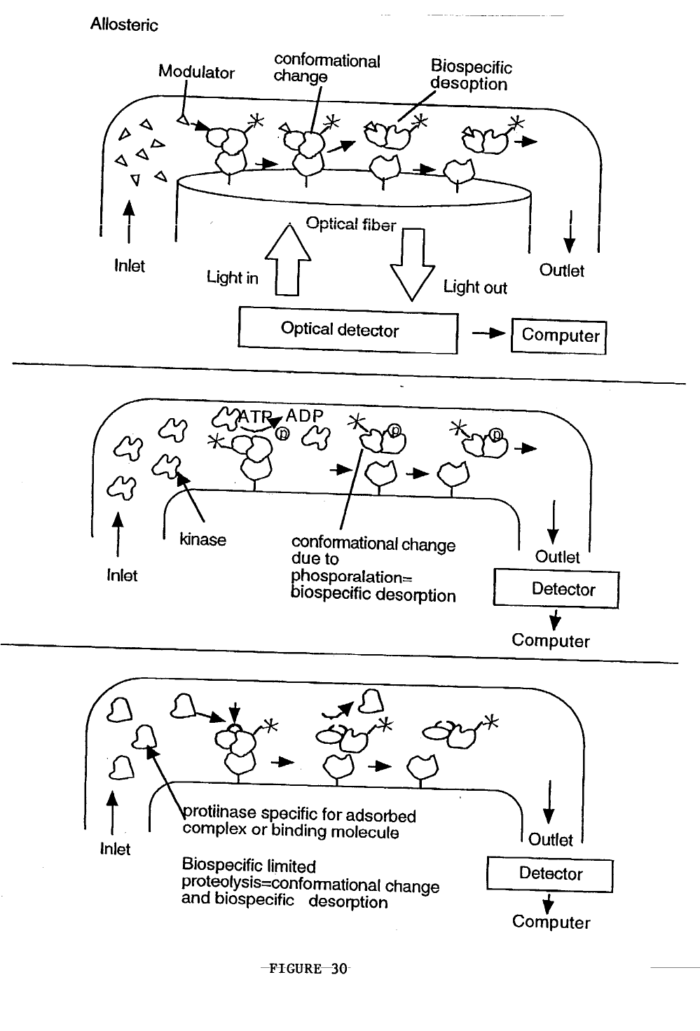

FIG. 30 illustrates the microflow systems as applied to allosteric binding events.

### Example 55

In some embodiments, the invention provides a microfluidic biospecific desorption assay method for characterizing the binding site of a protein/polypeptide. In this method, a buffer flow is established through a microchannel in fluidic contact with an immobilized binding complex which has a first immobilized binding pair member and a second labeled binding pair member. One of the first or second members is preferably the protein bound to the other binding pair member via the binding site. The protein may be the labeled member or the immobilized member. The immobilized binding pair member may be immobilized by covalent or noncovalent bonds. A polypeptide having an amino acid subsequence of the protein is introduced into the buffer flow and the desorption of the label is detected. If the polypeptide contains the binding motif, the labeled binding member will be desorbed and the binding site will thereby be localized to the portion of the protein having the amino acid sequence of the polypeptide.

These above steps can be repeated for each of a plurality of polypeptides of differing amino acid sequences of the protein. Exemplary polypeptides may be from 5 to 20, 5 to 50, 10 to 100, 20 to 100, or 50 to 250 amino acids in length. The polypeptide may be fragment generated by cleavage of the protein itself. With a sufficiently complete sampling of the protein sequence, at least one polypeptide would comprise the binding site to allow the identification of the binding site sequence. Shortened polypeptide versions of a polypeptide found to comprise the binding site could then be so screened to further localize the sequences of the proteinbinding site.

In some embodiments, the protein would be an antigen and the binding member complex would comprise the antigen and an antibody directed toward the antigen. The method, in that instance, would serve to characterize or identify the amino acid sequence of an epitope of the antigen.

In some embodiments, of the method, the binding pair complex comprises a polynucleotide bound to the protein and the binding site binds to the polynucleotide. The polynucleotide may be double stranded or single stranded DNA or RNA. In another embodiment, the binding pair may include a protein subject to post-translational modification, such as by the addition of a methyl group, or sugar or oligosaccharide moiety to the protein). 1

In some embodiments, the label is fluorescent, colored, radioactive, enzymatic, or chemiluminescent. In other embodiments, the detecting is by a biosensor such as a piezoelectric crystal, a surface plasmon resonance system, an acoustic wave sensor device, a fluorescence detector or a proximity scintillation surface.

### Example 56

In some embodiments, the invention provides an integrated microfluidic system for performing competitive displacement studies of a protein binding site. An exemplary system includes (a) a plurality of addressed reaction microchannels having a first immobilized binding pair member, an inlet for receiving a sample and a discharge outlet, and a second labeled binding pair member which is reversibly bound to the first member to form an immobilized complex. At least one of the first and second members is the protein and wherein the first and second members are bound via the binding site; (b) and optionally a plurality of sample polypeptides each having an (preferably known) amino acid subsequence of the protein, and preferably at least one or more of the polypeptides comprise the binding site; so that the absence or presence of a binding site can serve to localize the position of the binding site on the protein; and (c) a means for separately inputting at least one of each sample polypeptide into the sample inlet of at least one of each reaction microchannel; a means for inputting fluid from a buffer reservoir into each microchannel; (e) a detection system for each reaction microchannel which detects or monitors any dissociation of the complex; and (t) waste reservoir in fluid connection with the discharge outlet.

In some further embodiments, the label is fluorescent, colored, radioactive, enzymatic, or chemiluminescent. In other embodiments, the detection system comprises a biosensor selected from the group consisting of a piezoelectric crystal, a surface plasmon resonance system, an acoustic wave sensor device, a fluorescence detector or a proximity scintillation surface. Exemplary polypeptides may be from 5 to 20, 5 to 50, 10 to 100, 20 to 100, or 50 to 250 amino acids in length. The polypeptide may be fragment generated by cleavage of the protein itself. The protein may be the labeled or immobilized member and may be an antigen or an antibody.

In some embodiments, the label is fluorescent, colored, radioactive, enzymatic, or chemiluminescent. In other embodiments, the detection system comprises a biosensor selected from the group consisting of a piezoelectric crystal, a surface plasmon resonance system, an acoustic wave sensor device, a fluorescence detector or a proximity scintillation surface.

### Example 57

The following examples exemplify the use of biospecific desorption competitive displacement microflow systems and methods in various applications.

In another embodiment, the invention provides biospecific disorption or competitive displacement microflow systems and methods employing immobilized prebound members of binding pairs or complexes for identifying binding sites and screening for inhibitors of biospecific binding of biopolymers. These complexes can include protein-protein, protein-nucleic acid protein-drug, protein-carbohydrate, protein-carbohydrate and biological entities (e.g cells, viruses). For instance, microflow systems and methods for determining the ability of a sample to displace a member of a binding pair or complex can have a microchannel for receiving and conducting a fluid containing the sample; a first binding member immobilized in the microchannel, the first member being prebound to the channel and bound to a second binding member to form the complex and wherein the complex is positioned to contact the fluid; a detector for monitoring the desorption of the second binding member due to contact with the fluid whereby the ability to detect the desorbed entity is determined.

In some embodiments, the microflow system and method are used in mapping functional binding sites on the surfaces of proteins and nucleic acids, for instance, by (a) providing a binding pair or complex in a microflow reaction channel or capillary wherein one member of the pair or complex is immobilized in the flow passage (by covalent or noncovalent immobilization, e.g. biotin-avidin technology) and the other member of the pair or complex is labeled (e.g. with a fluorescent tag) and is bound to its immobilized binder (b) flowing a liquid sample containing biopolymers (e.g. peptides, oligonucleotides) corresponding (e.g., complementary in binding sequence or structure, or identical in sequence or surface structure) to the amino acid sequence of the bound proteins or oligonucleotides corresponding to the sequence of the immobilized nucleic acid; one or more samples; each sample would have a different peptide or protein fragment (corresponding to a bound protein) or oligonucleotide (corresponding to bound nucleic acid) through the microflow passage bearing the binding complex (c) allowing biopolymers corresponding to the binding sites on the binding pair or complex to biospecifically desorb (e.g., competitively displace) the binders (d) detecting the displaced binders with a detector, and (e) identifying the binding sites on the protein/and or nucleic acid from the known sample causing the biospecific desorption.

In other embodiments, a microflow system and method employ biospecific desorption to screen for inhibitors of biospecific interactions (e.g. protein-protein, virus-cell, bacteria-cell, protein-nucleic acid, protein-drug/therapeutic ligand, cell-cell, etc) by (a) providing a binding pair or complex in a microflow channel or capillary wherein one member of the pair or complex may be labeled; (b) flowing a liquid sample containing a possible inhibitor of the biospecific interaction in the microflow reaction channel through the reaction channel; in this fashion one or more samples, each containing a different potential inhibitor can be contacted with the complex by flowing them one at a time, through the reaction channel. Each sample is optionally flowed from a unique reservoir through the microflow channel bearing the binding complex; allowing samples to desorb the binders; and (c) detecting the desorbed binders with a detector; and (d) identifying the inhibitor from the known sample causing a desorption and thereby inhibiting the biospecific interaction.

In other embodiments, the microflow system and method employ biospecific desorption for epitope mapping by (a) immobilizing an antibody or protein antigen in a microflow channel (b) binding the protein antigen or antibody which may be labeled to the immobilized cognate binder (c) flowing one or a series of samples each containing a unique peptide corresponding to a different portion of the amino acid sequence of the protein antigen through the reaction channel one at a time; a set of peptides patterned on the amino acid sequence of the protein antigen is may hence be flowed through the reaction channel, one at a time; and (d) detecting or monitoring the biospecific desorption of the labeled binder with a detector, and (e) identifying the epitope on the protein from the peptide causing the biospecific desorption.

In other embodiments, the microflow system and method employ biospecific desorption to identify co- and post-translational modifications (e.g phosphorylated residues such as tyrosine phosphate, serine phosphate and threonine phosphate), lipid modified residues, carbohydrate modified residues and the like) on proteins by (a) immobilizing a binder (antibody, receptor, carbohydrate, protein or aptamer) that specifically and reversibly binds a modified amino acid in a microflow reaction channel; (b) binding a labeled analog of the modified amino acid (e.g. fluorescently labeled peptide bearing a tyrosine phosphate bound to an immobilized protein which binds tyrosine phosphate to the immobilized binder; (c) flowing a sample containing the protein or fragment thereof to be analyzed through the reaction microchannel; (d) detection of the biospecifically desorbed labeled analog with a detector and; and (e) identifying the modified amino acid from the biospecific desorption of the labeled analog.

In some embodiments of the above, the invention provides a kit comprising various amino acids or peptides bearing a post-translational modification for use in displacing a protein being studied to determine if it has such modifications. In some kits, a microfluidic array is provided (e.g., as described below) in which the various amino acids or peptides bearing the post-translational modification are a member of the immobilized binding complex and whose binding in the complex is biospecific for such modifications. The kits may further comprise buffer ingredients or buffer reservoirs.

In some further embodiments, the assay is performed in an array format in which a plurality of binding complexes are each located in a microchannel to form an array of reaction sites for screening a protein or protein fragment for post-translational modifications. The array would therefore comprise a plurality of binding complexes in which each one of the binding pair members bears a different post-translational modification. The binding pair member may be a protein, polypeptide, or amino acid bearing the modification and may be either an immobilized or labeled member. In preferred embodiment, the labeled member bears the modified amino acid. The array would then have a means for flowing a buffer containing the sample protein or polypeptide through the microchannels which are in fluidic contact with their prebound complexes. The displacement or desorption of the prebound complex(es) due to the contact with sample is then detected and a post-translational modification(s) of the sample protein or polypeptide is thereby identified. More than one sample protein or polypeptide could be flowed through sequentially. Such post-translational modifications include amidations, methylations, hydroxylations, phosphorylations, acetylations, oxidations, and the addition of sugar or lipid moieties.

In another embodiment, the invention provides a microflow system and method for identifying functional binding motifs in proteins by (a) binding a labeled peptide bearing the functional binding motif (e.g. fluorescently labeled) to an immobilized cognate binder; (b) flowing the protein or a fragment thereof containing the putative functional binding motif through the reaction microchannel; and (c) detecting the biospecific desorption caused by a protein flowing through the microflow reaction channel, whereby the polypeptide/peptide bearing the functional binding motif is identified.

In another embodiment, the microflow system and method is used to identify binding sites on protein-DNA or protein-RNA complexes by (a) immobilizing the DNA or RNA in the reaction microchannel (b) contacting the protein so that it binds to the nucleic acids; and (c) flowing oligonucleotides patterned on the sequence of the nucleic acid bound in the reaction channel one at a time through the reaction channel and monitoring the desorption of the protein so as to identify the oligonucleotide causing the desorption as the one having the protein binding sequences or alternatively (c) flowing peptides modeled on the amino acid sequence of the proteins one at a time through the reaction microchannel and monitoring the desorption of the protein so as to identify the polypeptide causing the desorption as the one having the protein binding site. In one embodiment, the amino acid sequences of the protein or polypeptide are each known a priori. In another embodiment, the polypeptides are fragments generated by hydrolysis of the protein and the sequences are later determined.

In another embodiment, the microflow system and method identify modulators of binding (e.g. binding as a function of phosphorylation or limited proteolysis; putative drugs or bioactive agents working by interacting with the binding site) by (a) immobilizing a binding pair or complex in a microflow channel (b) flowing a sample containing a potential binding modulator (e.g. a kinase along with ATP to add phosphate to a protein or a phosphatase to remove phosphate) through the reaction channel thus phosphorylating certain tyrosines or other amino acids or removing phosphates (c) detecting the amount of desorbed binder and (d) deducing there from the binding as a function of tyrosine phosphorylation.

In some exemplary embodiments of the above applications, the desorption studies are conducted in parallel using an array of microchannels bearing prebound complexes. For instance, an integrated microfluidic amino acid analysis system for performing competitive displacement studies, can have (a) a plurality of reaction microchannels, wherein each microchannel has a first binding pair member immobilized therein and an inlet for receiving a sample and a discharge outlet, (b) a second labeled binding pair member reversibly bound to the first and forming an immobilized complex; (c) at least one reservoir for input to said microchannels, wherein said reservoir is in fluid connection to at least one microchannel; (d) a means for inputting fluid from the reservoir to each microchannel; (e) a means for inputting sample into each microchannel; (f) a detection system for each reaction microchannel, said detection system detecting a product of the dissociation of the complex; and (g) a waste reservoir in fluid connection with said discharge outlet.

In some embodiments, the label is fluorescent, colored, radioactive, enzymatic, or chemiluminescent. In other embodiments, the detection system comprises a biosensor selected from the group consisting of a piezoelectric crystal, a surface plasmon resonance system, an acoustic wave sensor device, a fluorescence detector or a proximity scintillation surface.

All references cited in this specification, including the background, the summary, and the detailed description of the invention, are herein incorporated by reference in their entireties and to the extent that there is no inconsistency with the present disclosure.

What is claimed is:

1. A microfluidic biospecific desorption assay method for characterizing the binding site of a protein, said method comprising:
(1) establishing a buffer flow through a microchannel in fluidic contact with an immobilized binding complex comprising a first immobilized binding pair member and a second labeled binding pair member; wherein one of the first or second members is the protein or a fragment of the protein; and wherein the protein or protein fragment is bound to the other binding pair member via the binding site;
(2) introducing a polypeptide into the buffer flow; wherein the polypeptide has an amino acid subsequence of the protein;
(3) detecting the desorption of the label following introduction of the polypeptide; and
repeating steps (2) and (3) for each of a plurality of polypeptides of differing amino acid sequences, wherein at least one of the polypeptides comprises the binding site; whereby the polypeptide comprising the binding site is identified and the binding site is thereby localized to a portion of the protein having the amino acid sequence of the polypeptide comprising the binding site.

2. The method of claim 1, wherein the protein is an antigen, the binding member complex comprises the antigen and an antibody directed toward the antigen; and the binding site is an epitope of the antigen.

3. The method of claim 1, wherein the binding pair complex comprises a polynucleotide.

4. The method of claim 3, wherein the polynucleotide is DNA.

5. The method of claim 3, wherein the polynucleotide is RNA.

6. The method of claim 1, wherein the binding pair complex comprises an oligosaccharide.

7. The method of claim 1, wherein the protein is labeled.

8. The method of claim 1, wherein the protein is immobilized.

9. The method of claim 1, wherein the immobilized binding pair member is immobilized by covalent or noncovalent bonds.

10. The method of claim 1, wherein the polypeptide is from 5 to 20 amino acids in length.

11. The method of claim 1, wherein the polypeptide is from 20 to 100 amino acids in length.

12. The method of claim 1, wherein the polypeptide is from 50 to 250 amino acids in length.

13. The method of claim 1, wherein the polypeptide is a fragment of the protein.

14. The method of claim 1, wherein the label is fluorescent, colored, radioactive, enzymatic, or chemiluminescent.

15. The method of claim 1, wherein said detection system comprises a biosensor selected from the group consisting of a piezoelectric crystal, a surface plasmon resonance system, an acoustic wave sensor device, a fluorescence detector or a proximity scintillation surface.

16. An integrated microfluidic system for performing competitive displacement studies of a protein binding site, comprising:
(a) a plurality of addressed reaction microchannels, wherein each microchannel has a first binding pair member immobilized therein and an inlet for receiving a sample and a discharge outlet, and wherein a second labeled binding pair member is reversibly bound to the first member to form an immobilized complex therewith, wherein one of the first and second members is the protein and wherein the first and second members are bound via the binding site;
(b) a plurality of sample polypeptides, wherein each polypeptide has an amino acid subsequence of the protein, and wherein at least one polypeptide of the plurality comprises the binding site;
(c) a means for separately inputting at least one of each sample polypeptide into the sample inlet of at least one of each reaction microchannel;
(d) a means for inputting fluid from a buffer reservoir into each microchannel;
(e) a detection system for each reaction microchannel, said detection system detecting a product of the dissociation of the complex;
(f) a waste reservoir in fluid connection with the discharge outlet.

17. The system of claim 16, wherein the label is fluorescent, colored, radioactive, enzymatic, or chemiluminescent.

18. The system of claim 16, wherein said detection system comprises a biosensor selected from the group consisting of a piezoelectric crystal, a surface plasmon resonance system, an acoustic wave sensor device, a fluorescence detector or a proximity scintillation surface.

19. The system of claim 16, wherein the polypeptide is from 20-200 amino acids in length.

20. The system of claim 16, wherein the polypeptide is from 10 to 100 amino acids in length.

21. The system of claim 16, wherein the polypeptide is from 5 to 50 amino acids in length.

22. A microfluidic biospecific desorption assay method for characterizing the binding motifs of proteins, said method comprising:
(1) establishing a buffer flow in a microchannel in fluidic contact with an immobilized binding complex comprising a first immobilized binding pair member and a second labeled binding pair member; wherein at least one of the first or second members is a protein of known amino acid sequence having the binding motif and wherein the protein is bound to the other member of the binding pair via the binding motif;
(2) introducing a fragment of the protein into the microchannel buffer flow; wherein the fragment is of known amino acid sequence; and wherein the fragment comprises a minority portion of the protein; and
(3) detecting the desorption of the labeled member; whereby the binding motif of the protein is located to within or without the portion.

23. The method of claim 22, wherein steps (2) and (3) are repeated for each of a plurality of different fragments of the protein, wherein at least one of the plurality of fragments comprises the binding motif; whereby the desorption of the labeled member upon contact with the fragment comprising the binding motif is detected and the binding motif of the first biopolymer is localized to a region of the protein corresponding to the known sequence of the fragment comprising the binding motif.

24. The method of claim 22, wherein the protein is an antigen, and the binding member complex comprises the antigen and an antibody directed toward the antigen.

25. An integrated microfluidic amino acid analysis system for performing competitive displacement studies, comprising:
(a) a plurality of reaction microchannels, wherein each microchannel has a first binding pair member immobilized therein and an inlet for receiving a sample and a discharge outlet,
(b) a second labeled binding pair member reversibly bound to the first and forming an immobilized complex;
(c) at least one reservoir for input to said microchannels, wherein said reservoir is in fluid connection to at least one microchannel;
(d) a means for inputting fluid from the reservoir to each microchannel;
(e) a means for inputting sample into each microchannel;
(f) a detection system for each reaction microchannel, said detection system detecting a product of the dissociation of the complex;
(g) a waste reservoir in fluid connection with said discharge outlet.

26. The system of claim 25, wherein the label is fluorescent, colored, radioactive, enzymatic, or chemiluminescent.

27. The system of claim 25, wherein said detection system comprises a biosensor selected from the group consisting of a piezoelectric crystal, a surface plasmon resonance system, an acoustic wave sensor device, a fluorescence detector or a proximity scintillation surface.
